SEQUENCE LISTING (7 sequences)

SEQ ID NO: 1 - DNA, 40 bp, artificial sequence
  synthetic aptamer
gcgugcagugccuucggccgtgcggtgccuccgucacgct

SEQ ID NO: 2 - DNA, 40 bp, artificial sequence; identical to SEQ ID NO: 1
  synthetic aptamer

SEQ ID NO: 3 - DNA, 40 bp, artificial sequence; identical to SEQ ID NO: 1
  synthetic aptamer

SEQ ID NO: 4 - DNA, 40 bp, artificial sequence; identical to SEQ ID NO: 1
  synthetic aptamer

SEQ ID NO: 5 - DNA, 42 bp, artificial sequence
  synthetic aptamer
ggcgtgcagtgccttcggccgtgcggtgcctccgtcacgcct

SEQ ID NO: 6 - DNA, 41 bp, artificial sequence
  synthetic aptamer
ggcgtgcagtgccttcggccgtgcggtgcctccgtcacgcc

SEQ ID NO: 7 - DNA, 39 bp, artificial sequence
  synthetic aptamer
ggcgtgcagtgccggccgtgcggtgcctccgtcacgcct

(19) United States
(12) Patent Application Publication
(10) Pub. No.: US 2009/0203766 A1
(43) Pub. Date: Aug. 13, 2009

(54) vWF aptamer formulations and methods for use

(75) Inventors: James Gilbert, Bethlehem, CT (US); Renta Hutabarat, Winchester, MA (US); Robert G. Schaub, Pelham, NH (US)

Correspondence Address: MINTZ, LEVIN, COHN, FERRIS, GLOVSKYAND POPEO, P.C., ONE FINANCIAL CENTER, BOSTON, MA, 02111, US

(73) Assignee: Archemix Corp., Cambridge, MA (US)

(21) Appl. No.: 12/156,212

(22) Filed: May 30, 2008

Related U.S. Application Data
(60) Provisional application No. 60/932,701, filed on Jun. 1, 2007.
(60) Provisional application No. 60/933,893, filed on Jun. 8, 2007.
(60) Provisional application No. 60/936,483, filed on Jun. 19, 2007.
(60) Provisional application No. 60/958,938, filed on Jul. 9, 2007.
(60) Provisional application No. 60/961,871, filed on Jul. 24, 2007.
(60) Provisional application No. 60/965,629, filed on Aug. 20, 2007.
(60) Provisional application No. 61/000,531, filed on Oct. 25, 2007.
(60) Provisional application No. 61/001,705, filed on Nov. 2, 2007.
(60) Provisional application No. 61/005,684, filed on Dec. 7, 2007.
(60) Provisional application No. 61/009,929, filed on Jan. 2, 2008.
(60) Provisional application No. 61/011,517, filed on Jan. 17, 2008.
(60) Provisional application No. 61/124,647, filed on Apr. 18, 2008.

(51) Int. Cl. A61K 31/7088; A61K 48/00; A61K 31/4745

(52) U.S. Cl. 514/44 A; 514/165; 514/300; 514/171

(57) ABSTRACT

The invention relates to the formulation, dosing, administration and use of an aptamer antagonist therapeutic that binds to von Willebrand Factor.

### CROSS-REFERENCE TO RELATED APPLICATIONS

This non-provisional patent application claims priority to U.S. Provisional Patent Application Ser. No. 60/932,701, filed Jun. 1, 2007; U.S. Provisional Patent Application Ser. No. 60/933,893, filed Jun. 8, 2007; U.S. Provisional Patent Application Ser. No. 60/936,483, filed Jun. 19, 2007; U.S. Provisional Patent Application Ser. No. 60/958,938, filed Jul. 9, 2007; U.S. Provisional Patent Application Ser. No. 60/961,871, filed Jul. 24, 2007; U.S. Provisional Patent Application Ser. No. 60/965,629, filed Aug. 20, 2007; U.S. Provisional Patent Application Ser. No. 61/000,531, filed Oct. 25, 2007; U.S. Provisional Patent Application Ser. No. 61/001,705, filed Nov. 2, 2007; U.S. Provisional Patent Application Ser. No. 61/005,684, filed Dec. 7, 2007; U.S. Provisional Patent Application Ser. No. 61/009,929, filed Jan. 2, 2008; U.S. Provisional Patent Application Ser. No. 61/011,517, filed Jan. 17, 2008; and U.S. Provisional Patent Application Ser. No. 61/124,647, filed Apr. 18, 2008; each of which is incorporated herein by reference in its entirety.

### FIELD OF THE INVENTION

The invention relates to the formulation, dosing, administration and use of an aptamer antagonist therapeutic that binds to von Willebrand Factor (vWF).

### BACKGROUND OF THE INVENTION

Cardiovascular disease is the leading cause of death in the United States. Cardiovascular disease is a broad class of diseases that includes many different subsets of diseases. One such subset of diseases is thrombotic diseases. Thrombotic diseases or disorders are characterized by abnormal thrombus formation, which may lead to life-threatening events, such as heart attacks and strokes. Examples of diseases or disorders that involve abnormal thrombus formation include acute coronary syndrome (ACS); thrombotic microangiopathies (TMA); thrombotic thrombocytopenic purpura (TTP); von Willebrand's Disease, such as von Willebrand's Disease—type 2b (vWD-2b); and atherothrombosis, such as transient ischemic attack (TIA).

### ACS

Acute Coronary Syndrome (ACS) affects approximately 2 million people in the United States and includes the two forms of heart attack, Non-ST Segment Elevation Myocardial Infarction, or NSTEMI, and ST Segment Elevation Myocardial Infarction, or STEMI. A heart attack is caused by an arterial blockage or thrombus that reduces blood flow to the heart muscle. Both NSTEMI and STEMI patients are often managed with PCI, or angioplasty, a medical procedure that mechanically opens narrowed or clogged arteries to restore normal blood flow in the arteries.

Heart attack patients undergoing PCI receive a regimen of drugs, known as anti-thrombotics, which prevent the formation of additional harmful blood clots in their arteries during the procedure. This combination of anti-thrombotic drugs generally includes an anti-coagulant agent and an anti-platelet agent.

Current therapies for heart attack patients being treated with the combination of anti-thrombotic drugs and PCI have several limitations. Current approved anti-platelet agents include the class of drugs known as GPIIb/IIIa antagonists, which target and bind to a site on platelets known as the GPIIb/IIIa receptor. By binding to the GPIIb/IIIa receptor, these drugs prevent platelets from aggregating and forming a thrombus. GPIIb/IIIa inhibitors include the approved drugs REOPRO® and INTEGRILIN®. GPIIb/IIIa antagonists have the following limitations:
Limited effect on myocardial perfusion. Clinical data demonstrate that GPIIb/IIIa antagonists do not improve blood flow in the microvasculature in heart attack patients undergoing PCI. Data published in numerous medical journals show a direct correlation between poor myocardial perfusion and a decrease in a patient's subsequent cardiac function and survival. Accordingly, there is an unmet medical need for a drug that improves blood flow in the microvasculature of patients undergoing PCI.
Bleeding risk. Because GPIIb/IIIa antagonists suppress platelet function independent of shear force, these drugs are active in the veins and arteries throughout the body, which is beyond the region of the primary blood clot. Accordingly, there is an increased risk of significant bleeding in the systemic circulation in patients receiving GPIIb/IIIa antagonists.
Inconvenient dosing regimen. Both REOPRO® and INTEGRILIN® have been approved based on a regimen of an immediate intravenous injection, or bolus, followed by an extended period of intravenous infusion. This dosing regimen is inconvenient. INTEGRILIN® is approved for administration as a bolus during PCI with a continuous intravenous infusion thereafter for 18 to 24 hours, while REOPRO® is approved based upon a post-procedural infusion of 12 hours.

In addition, PCI procedures are normally successful in restoring blood flow in the larger, primary arteries of the heart. However, similar to the GPIIb/IIIa antagonists, PCI does not target the microvasculature.

### TMA/TPP

Thrombotic microangiopathy (TMA) describes syndromes of microangiopathic hemolytic anemia, thrombocytopenia and variable signs of organ impairment due to platelet aggregation in the microcirculation. Examples of TMA include thrombotic thrombocytopenic purpura and hemolytic uremic syndrome. Hemolytic uremic syndrome (HUS) describes childhood cases of TMA dominated by renal impairment, while the term thrombotic thrombocytopenic purpura (TTP) refers to adult cases of TMA with predominant neurological abnormalities. Examples of TTP include secondary TTP, idiopathic TTP and congenital TTP (which is also known as hereditary TTP or Upshaw-Schulman syndrome). There is no approved drug treatment for patients with TMA or TPP.

vWD-2b

Type 2b von Willebrands Disease (vWD-2b) is an inherited disorder that is characterized by defective von Willebrand Factor (vWF).

### Atherothrombosis

The disease category atherothrombosis includes transient ischemic attack, stroke and myocardial infarction. High risk atherothrombosis patients are those who have suffered a transient ischemic attack, or TIA, which is a temporary blockage of a cranial artery. Often referred to as “mini-strokes,” TIA's are transient and usually do not inflict permanent damage, but are often a pre-cursor for a stroke.

vWF

Medications are needed to treat these life-threatening disorders that are caused by abnormal thrombus formation. Currently, none of the existing anti-thrombotic drugs target vWF.

von Willebrand Factor (vWF) plays a role in thrombosis, hemostasis and disease. It is a large multi-subunit, multimeric soluble factor critical to normal hemostasis. vWF is expressed by vascular endothelial cells where it is stored in intracellular vesicles and released into the subendothelial extracellular matrix and circulating blood. Some of the vWF in circulating blood is taken up and stored in platelet alpha granules for release upon platelet activation (Ruggeri Z M, J Thromb Haemost, 1 (7): 1335-42 (2003)). Normally, vWF stabilizes Factor VIII and promotes platelet adhesion at wound sites.

vWF has distinct domains that bind key modulators of hemostasis and thrombosis. vWF binds collagen via its A3 domain, platelets via its A1 or C3 domains, and forms homotypic multimers between matrix-bound and soluble vWF molecules (Savage B, et al., Proc Natl Acad Sci, 99 (1):425-30 (2002)).

### ACS

vWF is involved in platelet adhesion, activation and aggregation, and plays a pivotal role in hemostasis and in the formation of blood clots. Under the conditions of high shear force present in the arterial circulation, vWF is activated by means of a physical deformation that exposes its A1 domain and enables binding to the platelet GPIb receptor (Siedlecki et al., Blood, 88 (8):2939-50 (1996)). Activated vWF binds to cellular elements in the blood known as platelets, which play a key role in the normal process of blood clotting. vWF captures platelets from the flowing bloodstream, causing the platelets to adhere to the blood vessel wall. This adhesive interaction between vWF and platelets activates the bound platelets and causes them to recruit additional platelets from the bloodstream. These recruited platelets aggregate on the blood vessel wall and form the beginning of a blood clot. As the primary blood clot grows and shear force within the artery is further increased, more vWF is activated, enabling the formation of new clots. These new clots break off and lodge in the smaller, distal vessels of the heart known as the microvasculature, where they may join other clots that have formed in response to local activation of vWF. Together with the primary clot, these smaller clots restrict the normal process of delivering, or perfusing, blood to the working heart muscle, or myocardium, causing a heart attack.

vWF-dependent platelet adhesion, aggregation and activation can also occur at sites of vascular injury and endothelial denudation, where exposed and activated vWF can promote thrombogenesis (Blann, Thromb Haemost, 95 (1):49-55 (2006)). Fluid-phase vWF, secreted by activated endothelium and platelets in response to biochemical signals, such as thrombin and epinephrine that accompany ischemia, interacts to form multimers with subendothelial matrix-bound vWF and thereby augments vWF-mediated platelet adhesion and aggregation (Ruggeri 2003). Shear stress in conduit arteries can be elevated at the sites of stenosis (Mailhac A, et al., Circulation, 90 (2):988-96 (1994); Siegel J M, et al., J Biomech Eng, 116 (4):446-51 (1994); and Strony J, et al., Am J Physiol, 265 (5 Pt 2):H1787-96 (1993)), leading to activation of vWF, triggering vWF-platelet binding, and generating pro-coagulant platelet-derived microparticles (Jackson S P, et al., Blood, 107 (9):3418-9 (2006); Reininger A J, et al., Blood, 107 (9):3537-45 (2006)). Substantial shear stress is present even down to the arteriolar level of the circulatory tree (Stepp D W, et al., Circulation, 100 (14):1555-61 (1999); Tangelder G J, et al., Am J Physiol, 254 (6 Pt 2):H1059-64 (1988)), and is apparently sufficient to activate the vWF that is expressed by “stressed” or injured vascular endothelium and promote thrombosis in the microcirculation (Sako D., “Evaluation of valine-substituted GPIbalpha-Ig fusion proteins as novel antithrombotic agents”, Arteriosclerosis, Thrombosis and Vascular Biology (2007, in press). In the absence of high shear force, e.g., in the capillary or venous circulation, vWF is not activated and its contribution to thrombogenesis is greatly reduced (Blann 2006; Ruggeri 2003).

Circulating plasma levels of vWF are chronically elevated in the clinical setting of endothelial dysfunction and atherosclerosis (Blann A D, Pathophysiol Haemost Thromb, 33 (5-6):256-61 (2003); Paramo J A, et al., J Thromb Haemost, 3 (4):662-4 (2005)), and acute elevations are observed in acute coronary syndromes (Collet J P, et al., Circulation, 108 (4):391-4 (2003); Lee et al., Blood, 105 (2):526-32 (2005); Montalescot G, et al., Circulation, 98 (4):294-9 (1998); and Ray K K, et al., Eur Heart J, 26 (5):440-6 (2005)). Elevated vWF is considered to be both a prognostic marker and a pathophysiologic mediator of adverse outcomes in heart disease (Becker, Eur Heart J, 26 (5):421-2 (2005)), and normalization of vWF activity levels may represent a new therapeutic paradigm in cardiovascular medicine.

In addition to its use as an effective anti-thrombotic therapeutic principle, vWF antagonism offers an improved risk-to-benefit ratio in comparison to GPIIb/IIIa receptor antagonism for use in the management of ACS and in conjunction with percutaneous coronary intervention (PCI) (De Meyer et al., Cardiovasc Hematol Disord Drug Targets, 6 (3):191-207 (2006); Vanhoorelbeke K, et al., Curr Drug Targets Cardiovasc Haematol Disord, 3 (2):125-40 (2003)). A monoclonal antibody antagonist of vWF was found to inhibit thrombosis without inducing bleeding in pre-clinical studies (Eto K, et al., Arterioscler Thromb Vasc Biol, 19 (4):877-82 (1999); Kageyama S, et al., Br J Pharmacol, 122 (1):165-71 (1997); Kageyama et al., Arherioscler Thromb Vasc Biol, 22 (1):187-92 (2002); Kageyama et al., Thromb Res, 101 (5):395-404 (2001); Kageyama et al., Arterioscler Thromb Vasc Biol, 20 (10):2303-8 (2000); and Yamamoto, Thromb Haemost, 79 (1):202-10 (1998)) and in a healthy volunteer human study (Machin S J., J Thrombosis Haemostasis, Supplement 1:OC328 (2006)). The dissociation of anti-thrombotic efficacy from prolongation of cutaneous bleeding time (CBT), a proxy for risk of bleeding, was also observed in a primate model in which ARC1779 was compared directly to the GPIIb/IIIa antagonist, abciximab (REOPRO®).

### TMA/TTP

TTP is a rare blood disorder caused by elevated levels of activated von Willebrand Factor (vWF) in the blood that is due to a deficiency of the enzyme responsible for vWF degradation. This enzyme, known as ADAMTS13, is responsible for vWF degradation, which is necessary to maintain the normal balance between bleeding and clotting. There are two forms of TTP, an inherited form and an acquired form. The inherited form is caused by mutations in the ADAMTS13 gene that impair the normal function of the enzyme. Patients with the acquired form of TTP do not have mutations in this gene, but instead produce antibodies that block the activity of the ADAMTS13 enzyme. A deficiency of ADAMTS13 or the absence of this enzyme results in excessive levels of activated vWF that cause platelet aggregation, resulting in widespread blood clotting, which can lead to life-threatening events, such as heart attack and stroke.

In patients suffering from TTP, platelets bind together abnormally and adhere to the walls of blood vessels, forming clots throughout the body. As these clots grow in size and multiply, they restrict blood flow to critical organs, such as the brain, kidneys and heart, potentially causing stroke, seizure, kidney failure or heart attack. These events trigger acute episodes of disease resulting in hospitalization. TTP is a syndrome that is characterized by microangiopathic hemolytic anemia, thrombocytopenia, neurologic abnormalities, fever and renal dysfunction.

Each year in the United States, between four and 11 new cases of TTP per million of the total population are diagnosed. There is no drug treatment currently approved for patients with TTP. Patients are managed in the hospital by removing and replacing their plasma with fresh plasma from donors, which is known as plasma exchange. Although plasma exchange results in reduced mortality, it is an expensive and invasive procedure. In addition, time to response is slow and many complications can occur, such as death, systemic infection, thrombosis, hemorrhage, hypotension, anaphylaxis, serum sickness, hypoxia and vomiting. Even with plasma exchange, acute episodes of TTP are associated with a high mortality rate, estimated to be as high as 20%. Even in non-fatal cases there can be serious medical consequences, such as strokes, seizures, kidney failure and heart attack. Studies estimate that neurologic symptoms are present in 60% of patients upon initial examination and ultimately develop in about 90% of patients, while approximately 20% of acute TMA patients with no history of coronary artery disease suffered myocardial infarction.

### vWD-2b

Type 2b von Willebrand's Disease is characterized by excessive platelet binding, which is caused by constitutively active vWF. There is an unmet need for the treatment of women with vWD-2b. These women experience excessive menses and thrombocytopenia that often leads to hospitalization. Standard treatment for this condition exacerbates the thrombocytopenia and can cause anemia.

### Atherothrombosis

Atherothrombosis is a result of elevated and activated vWF. This is due to the fact that endothelial injury and shear forces in atherosclerotic arteries lead to vWF secretion and activation, which promotes platelet adhesion, activation and aggregation.

Thus, there is a need for therapeutics that target vWF and offer alternatives and advantages to current thrombotic therapies.

### SUMMARY OF THE INVENTION

The present invention provides pharmaceutical compositions, or formulations, of aptamers that bind to von Willebrand Factor (vWF), referred to herein as “vWF aptamers”, and methods for using such vWF aptamers to treat vWF-mediated diseases and disorders, including the treatment of thrombotic disorders involving vWF-mediated platelet aggregation. The formulations comprise a vWF aptamer or a pharmaceutically acceptable salt thereof and a pharmaceutically acceptable solvent. The formulations and dosages described herein are designed to maximize clinical efficacy in the treatment of thrombotic disorders while simultaneously decreasing or minimizing adverse side effects, such as bleeding and/or complement activation.

The formulations described herein comprise a vWF aptamer or a pharmaceutically acceptable salt thereof. The formulations may comprise any aptamer that binds to vWF or a variant or a fragment thereof. Preferably, the aptamer binds the A1 domain of vWF. More preferably, the aptamer binds both the full length vWF and the A1 domain.

Preferably, the vWF aptamer binds to vWF or a fragment thereof and acts as an antagonist to inhibit the function of vWF.

Preferably, the vWF aptamer is ARC1779. ARC1779 is a synthetically manufactured, modified DNA/RNA aptamer that is conjugated to a polyethylene glycol (PEG, 20 kDa) moiety at the 5′-terminus.

ARC1779 is an aptamer having the following structure:

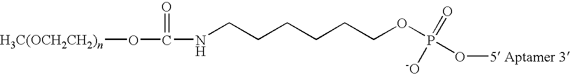

wherein: “n” is about 454 ethylene oxide units (PEG=20 kDa) and the aptamer comprises the following nucleic acid sequence or fragment thereof:
mGmCmGmUdGdCdAmGmUmGmCmCmUmUmCmGmGmCdCmG-s-dTmGdCdGdGdTmGmCdCmUdCdCmGmUdCmAmCmGmC-3T (SEQ ID NO: 1), wherein “cm” is a 2′-OMe substituted nucleotide, “d” is a deoxyribonucleotide, “s” is a phosphorothioate internucleotide linkage and “3T” is an inverted deoxythymidine.

The formulations may comprise any amount of vWF aptamer. Typically, the formulations comprise 1-50 mg/ml vWF aptamer or a pharmaceutically acceptable salt thereof. For example, suitable concentrations of vWF aptamer or a pharmaceutically acceptable salt thereof include, but are not limited to, 1-50 mg/ml or any 0.1 mg/ml increment thereof. Preferably, intravenous formulations comprise 5-25 mg/ml of vWF aptamer or a pharmaceutically acceptable salt thereof. More preferably, intravenous formulations comprise 5-15 mg/ml of vWF aptamer or a pharmaceutically acceptable salt thereof. Most preferably, intravenous formulations comprise 10 mg/ml of vWF aptamer or a pharmaceutically acceptable salt thereof. Alternatively, subcutaneous formulations preferably comprise 25-50 mg/ml of vWF aptamer or a pharmaceutically acceptable salt thereof. More preferably, subcutaneous formulations comprise 40-50 mg/ml of vWF aptamer or a pharmaceutically acceptable salt thereof. Most preferably, subcutaneous formulations preferably comprise 50 mg/ml of vWF aptamer or a pharmaceutically acceptable salt thereof. Preferably, subcutaneous formulations are low volume formulations.

The formulations also comprise a pharmaceutically acceptable solvent. Preferably, the pharmaceutically acceptable solvent is selected from the group consisting of 0.9% saline (physiological saline or sterile isotonic saline solution) or phosphate buffered saline. Most preferably, the pharmaceutically acceptable solvent is 0.9% saline.

The formulations may comprise any amount of pharmaceutically acceptable solvent. Preferably, subcutaneous formulations comprise 0.1-2 ml of pharmaceutically acceptable solvent. For example, suitable subcutaneous formulations include, but are not limited to, 0.1-2 ml of pharmaceutically acceptable solvent or any 0.1 ml increment thereof. Most preferably, subcutaneous formulations comprise 1 ml or less of pharmaceutically acceptable solvent. Alternatively, intravenous formulations may comprise any amount of pharmaceutically acceptable solvent.

Various embodiments of the formulations may, optionally, include one or more of the following: buffer, pH adjuster, tonicity agent, cosolvent or pharmaceutically acceptable carrier.

The dose of vWF aptamer is administered so as to achieve and then maintain a steady state blood concentration equal to at least the EC₉₀, and preferably of 2-12 μg/ml. The steady state blood concentration can be any concentration between, and including, 2 μg/ml and 12 μg/ml in increments of 0.1 μg/ml. Preferably, the dose achieves a steady state blood concentration of 3-10 μg/ml. More preferably, the dose achieves a steady state blood concentration of 3-8 μg/ml. Most preferably, the dose achieves a steady state blood concentration of 3-6 μg/ml.

The dose of vWF aptamer is administered in mg per kg (mg/kg) of body weight. The daily dose of vWF aptamer is 0.05-10 mg/kg. The daily dose can be any dose between, and including, 0.05 mg/kg and 10 mg/kg in increments of 0.01 mg/kg. This dosage may be administered in a single dose, multiple doses, as a continual dose or a combination thereof.

Preferably, in order to treat TMA/TPP, once the desired blood concentration is reached, a dose of vWF aptamer of 0.384-2.592 mg/kg is administered on a daily basis. More preferably, a dose of 0.576-2.16 mg/kg is administered on a daily basis. Even more preferably, a dose of 0.576-1.728 mg/kg is administered on a daily basis. Most preferably, a dose of 0.576-1.296 mg/kg is administered on a daily basis. This dosage may be administered in a single dose, multiple doses, as a continual dose or a combination thereof. For example, a loading dose or doses may be administered followed by a maintenance dose or doses. To achieve the desired blood concentration level, any dose of 0.05-10 mg/kg can be used.

Preferably, the formulations are administered with a loading dose, a maintenance dose and a tapered dose.

Preferably, the loading dose is an intravenous infusion of vWF aptamer in a dose- and duration-escalation design. For example, the loading dose may be administered in three ten-minute intervals over 30 minutes or three twenty-minute intervals over 60 minutes. In the first ten-minute or twenty-minute interval, a dose of X mg/kg is administered by constant infusion. In the second ten-minute or twenty-minute interval, a dose of 2×mg/kg is administered by constant infusion. In the third ten-minute or twenty minute interval, a dose of 4×mg/kg is administered by constant infusion. The sum of X, 2× and 4× equals the total loading dose. Preferably, the total loading dose is 0.09-0.56 mg/kg. More preferably, the total loading dose is 0.14-0.47 mg/kg. Even more preferably, the total loading dose is 0.14-0.37 mg/kg. Most preferably, the total loading dose is 0.14-0.28 mg/kg. However, the dose and duration may be varied to achieve essentially the same result of a safe and tolerable dose that rapidly achieves the desired steady state concentration.

Preferably, the maintenance dose of vWF aptamer is administered as a continual infusion at a constant rate. Preferably, the maintenance dose is administered at a rate of 0.0002-0.0018 mg/kg/mm, which is equal to 0.384-2.592 mg/kg/day, respectively. More preferably, the maintenance dose is administered at a rate of 0.0004-0.0015 mg/kg/min, which is equal to 0.576-2.16 mg/kg/day, respectively. Even more preferably, the maintenance dose is administered at a rate of 0.0004-0.0012 mg/kg/min, which is equal to 0.576-1.728 mg/kg/day, respectively. Most preferably, the maintenance dose is administered at a rate of 0.0004-0.0009 mg/kg/min, which is equal to 0.576-1.296 mg/kg/day, respectively. Preferably, the maintenance dose is administered as a continuous infusion until normalization of platelet count is achieved. Normalization is defined as a platelet count ≧150×10⁹ per liter (150,000 per μl) measured over three consecutive days.

Preferably, the maintenance dose of vWF aptamer is tapered by 50% (½ of the administration rate) on the next to last day of administration and by another 50% (½ of the administration rate to 25% of the total) on the last day of administration. The infusion will be stopped after the tapering procedure.

Alternatively, the formulation may be administered as the loading dose described above (dose- and duration-escalation design) followed by a subcutaneous injection.

In another embodiment and after plasma exchange, half of the original loading dose of vWF aptamer is administered over 30 minutes. Preferably, the formulation is administered 0-30 minutes, and more preferably 0-15 minutes, after plasma exchange. The purpose of this is to restore aptamer concentration to the target level. Then, the continuous infusion will resume.

Preferably, in order to treat ACS, a dose or doses of vWF aptamer are administered sufficient to maintain the desired blood level concentration, preferably through the pre-intervention period, more preferably through the pre-intervention and intervention periods, and most preferably through the pre-intervention, intervention and initial post-intervention periods. Preferably, a dose of 0.3-10 mg/kg is administered. More preferably, a dose of 0.3 mg/kg is administered. Even more preferably, a dose of 0.6 mg/kg is administered. Most preferably, a dose of 1.0 mg/kg is administered. Preferably, the formulation is administered as a bolus or a slow bolus over a 15 minute time period.

ARC1779 is manufactured for clinical use as a sterile isotonic saline solution (0.9% saline solution) for injection. Preferably, the formulation is provided in a 10 mg/mL solution. The formulation may be administered directly into an individual or may be diluted into an IV bag prior to administration.

The formulations are suitable for parenteral administration. Preferably, the formulations are administered subcutaneously. Most preferably, the formulations are administered intravenously.

The formulations may be administered parenterally, for example, as a bolus; a slow bolus over a short period of time, such as 15 minutes; a continual infusion or a continual drip. Preferably, the formulations are administered by continual infusion.

Administration by continual infusion may be at a constant rate. Alternatively, the rate of administration may be varied (not constant) over time in order to take into account loading doses prior to or at the beginning of administration and tapering of the infusion rate at the end of administration. Preferably, the rate of continual infusion is varied.

The aptamer formulations provided herein are administered to subjects, particularly, human subjects, in an amount effective to inhibit, reduce, block or otherwise modulate vWF-mediated platelet aggregation.

The formulations are used to treat, prevent or ameliorate vWF-mediated diseases and disorders, including the treatment of thrombotic disorders involving vWF-mediated platelet aggregation. The diseases and disorders to be treated, prevented or ameliorated are selected from the group consisting of: essential thrombocytopenia, thrombotic microangiopathies (TMA), thrombotic thrombocytopenic purpura (TTP), Type 2b von Willebrand's Disease, pseudo type 2b von Willebrand's Disease, peripheral artery disease, e.g., peripheral arterial occlusive disease, unstable angina, angina pectoris, arterial thrombosis, atherosclerosis, myocardial infarction, acute coronary syndrome (ACS), atrial fibrillation, carotid stenosis, unstable carotid lesions, cerebral infarction, cerebral thrombosis, ischemic stroke and transient cerebral ischemic attack. In some unstable carotid disease embodiments, the pharmaceutical composition of the invention is administered prior to, during and/or after carotid revascularization procedures, either percutaneously or surgically.

The formulations may be administered in combination with other drugs or therapies. For example, the formulations of the invention may be used in combination with plasma exchange, corticosteroids, immunosuppressives, aspirin, clopidogrel, or aspirin and clopidogrel for use in treating TMAs. By way of another example, the formulations may be administered in combination with aspirin, clopidogrel, or aspirin and clopidogrel for use in treating ACS and TMAs. As a further example, the formulations may be administered in combination with antibiotics for use in treating HUS. The formulations are also compatible with standard hypersensitivity regimens, such as corticosteroids and antihistamines.

The formulations can be packaged for use in a variety of pharmaceutically acceptable containers using any pharmaceutically acceptable container closure, as the formulations are compatible with PVC-containing and PVC-free containers and container closures.

The formulations may also be packaged in a kit.

In another embodiment of the invention, the use of ARC1779 in the manufacture of a medicament, pharmaceutical composition or formulation for the treatment, prevention or amelioration of a disease mediated by von Willebrand Factor is provided.

In yet a further embodiment of the invention, the use of ARC1779 in the manufacture of a medicament, pharmaceutical composition or formulation for the treatment, prevention or amelioration of a disease mediated by von Willebrand Factor is provided, wherein the medicament, pharmaceutical composition or formulation comprises ARC1779 and a 0.9% saline solution.

In yet a further embodiment of the invention, the use of ARC1779 in the manufacture of a medicament, pharmaceutical composition or formulation for the treatment, prevention or amelioration of a disease mediated by von Willebrand Factor is provided, wherein the medicament, pharmaceutical composition or formulation is for administration to a patient in a dosage amount of aptamer in the range of (i) 0.05 mg/kg to 10 mg/kg, preferably 0.384-2.592 mg/kg, more preferably 0.576-2.16 mg/kg, even more preferably 0.576-1.728 mg/kg and most preferably 0.576-1.296 mg/kg, or (ii) 0.5-2000 mg, preferably 3.84-518.4 mg, more preferably 5.76-432 mg, even more preferably 5.76-345.6 mg, and most preferably 5.76-259.2 for a 10-200 kg patient, after administration of a loading dose.

In another embodiment of the invention a medicament, pharmaceutical composition or formulation is provided comprising ARC1779 for use in the treatment, prevention or amelioration of a disease mediated by von Willebrand Factor.

In yet a further embodiment of the invention a medicament, pharmaceutical composition or formulation is provided comprising ARC1779 and a 0.9% saline solution for use in the treatment, prevention or amelioration of a disease mediated by von Willebrand Factor.

In yet a further embodiment of the invention a medicament, pharmaceutical composition or formulation is provided comprising ARC1779 in a dosage amount in the range (i) 0.05 mg/kg to 10 mg/kg, preferably 0.384-2.592 mg/kg, more preferably 0.576-2.16 mg/kg, even more preferably 0.576-1.728 mg/kg and most preferably 0.576-1.296 mg/kg, or (ii) 0.5-2000 mg, preferably 3.84-518.4 mg, more preferably 5.76-432 mg, even more preferably 5.76-345.6 mg, and most preferably 5.76-259.2 for a 10-200 kg patient, after administration of a loading dose, wherein the medicament, pharmaceutical composition or formulation is for use in the treatment, prevention or amelioration of a disease mediated by von Willebrand Factor.

### BRIEF DESCRIPTION OF THE DRAWINGS

FIG. 1 is a graph depicting mean cutaneous bleeding time (CBT) over time after single-dose, IV push administration of ARC1779 (Cohorts 1-3).

FIG. 2 is a graph depicting mean CBT over time after single-dose, IV slow bolus administration of ARC1779 (Cohorts 4-7).

FIG. 3 is a graph depicting mean C3a concentration over time after single-dose, IV push administration of ARC1779 (Cohorts 1-3).

FIG. 4 is a graph depicting C3a concentration over time in individual subjects after single-dose, IV push administration of ARC1779 at 0.3 mg/kg (Cohort 3).

FIG. 5 is a graph depicting mean C3a concentration over time after single-dose, slow bolus administration of ARC1779 (Cohorts 4-7).

FIG. 6 is a graph depicting mean ARC1779 plasma concentrations over time after single-dose, IV push administration (Cohorts 1-3).

FIG. 7 is a graph depicting mean ARC1779 plasma concentrations over time after single-dose, IV slow bolus administration (Cohorts 4-7).

FIG. 8 is a graph depicting the relationship between Cₘₐₓ and dose after single-dose, IV push administration.

FIG. 9 is a graph depicting the relationship between Cₘₐₓ and dose after single-dose, IV slow bolus administration.

FIG. 10 is a graph depicting the relationship between AUC_{(0-∞)} and dose after single-dose, IV push administration.

FIG. 11 is a graph depicting the relationship between AUC_{(0-∞)} and dose after single-dose, IV slow bolus administration.

FIG. 12 is a graph depicting mean vWF activity over time after single-dose, IV push administration of ARC1779 (Cohorts 1-3).

FIG. 13 is a graph depicting mean vWF activity over time after single-dose, slow bolus administration of ARC1779 (Cohorts 4-7).

FIG. 14 is a graph depicting mean PFA-100® closure time after single-dose, IV push administration of ARC1779 (Cohorts 1-3).

FIG. 15 is a graph depicting mean PFA-100® closure time after single-dose, IV slow bolus administration of ARC1779 (Cohorts 4-7).

FIG. 16 is a graph depicting concentration- and vWF activity-time profiles of ARC1779 after single-dose, IV push administration.

FIG. 17 is a graph depicting concentration- and vWF activity-time profiles of ARC1779 after single-dose, IV slow bolus administration.

FIG. 18 is a graph depicting the PK/PD relationship of vWF inhibition to ARC1779 concentration after single-dose, slow bolus administration of ARC1779 fitted to an Eₘₐₓ model (Cohorts 4-7).

FIG. 19 is a graph depicting concentration- and PFA-100®-time profiles of ARC1779 after single-dose, IV push administration.

FIG. 20 is a graph depicting concentration- and PFA-100®-time profiles of ARC1779 after single-dose, IV slow bolus administration.

FIG. 21 is a graph depicting PK/PD relationship of PFA-100® closure time to ARC1779 concentration after single-dose, IV slow bolus administration fitted to an Eₘₐₓ model (Cohorts 4-7).

FIG. 22 is a series of graphs depicting the cutaneous bleeding time (CBT) profiles as a function of time. FIG. 22A shows the CBT profile for ARC1779 administered via IV push. FIG. 22B shows the CBT profile for ARC1779 administered via IV slow bolus. FIG. 22C shows the CBT profile for ARC1779 administered via IV slow bolus+infusion.

FIG. 23 is a series of graphs depicting ARC1779 plasma concentration as a function of time. FIG. 23A shows the plasma concentration for ARC1779 administered via IV push. FIG. 23B shows the plasma concentration for ARC1779 administered via IV slow bolus. FIG. 23C shows the plasma concentration for ARC1779 administered via IV slow bolus+infusion.

FIG. 24 is a series of graphs depicting vWF activity as a function of time. FIG. 24A shows activity for vWF administered via IV push. FIG. 24B shows activity for vWF administered via IV slow bolus. FIG. 24C shows activity for vWF administered via IV slow bolus+infusion.

FIG. 25 is a series of graphs depicting PFA-100® closure time profiles as a function of time. FIG. 25A shows the profile for ARC1779 administered via IV push. FIG. 25B shows the profile for ARC1779 administered via IV slow bolus. FIG. 25C shows the profile for ARC1779 administered via IV slow bolus+infusion.

FIG. 26 is a graph depicting the ARC1779 concentration-activity relationship for ARC1779 administered via IV slow bolus.

FIG. 27 is a series of graphs depicting Eₘₐₓ modeling of the ARC1779 PK/PD relationship. FIG. 27A shows the percent vWF inhibition as a function of time. FIG. 27B shows the PFA-100® closure time as a function of ARC1779 concentration.

FIG. 28 shows the platelet response to the anti-von Willebrand Factor aptamer ARC1779 in a patient with acute refractory TTP. ARC1779 increased platelet counts in each period, and discontinuation of the infusion induced a reproducible profound decrease in platelet counts each time (red circles).

FIG. 29 is a series of graphs showing plasma concentrations of ARC1779 and their relation to platelets, lactate dehydrogenase (LDH) and creatinine levels.

FIG. 30 is a graph depicting the pharmacokinetic profile of ARC1779 following single IV bolus at 5, 10 and 20 mg/kg to male and female cynomolgus monkeys.

FIG. 31 is a graph depicting the pharmacokinetic profile of ARC1779 following IV bolus plus continuous infusion to male and female cynomolgus monkeys.

FIG. 32 is a graph depicting mean ARC1779 concentration profile vs. mean ARC1779 vWF activity as a function of time.

FIG. 33A is a diagram of the proposed secondary structure of ARC1779. ARC1779 is a synthetically manufactured, modified DNA/RNA aptamer that is conjugated to a polyethylene glycol (PEG, MW 20 kDa) moiety at the 5′-terminus. Black=2′-O-methyl substituted nucleotides; bold=2′-deoxyribonucleotides; ps=phosphorothioate internucleotide linkage; iT=inverted deoxythymidine. FIG. 33B is a graph depicting the binding isotherms for the oligonucleotide core of ARC1779. Trace 5′-³²P end-labeled aptamer was incubated with increasing concentrations of human VWF (squares) or human serum albumin (circles) in the presence of 0.1 mg/ml BSA. The ratio of bound aptamer to total aptamer was used to determine aptamer affinities for VWF.

FIGS. 34A-D illustrate that ARC1779 inhibits platelet adhesion at high shear rates. In FIG. 34A, human whole blood with labeled platelets was perfused over immobilized collagen at an arterial shear rate of 1,500/s. Quantification of the fluorescently covered area after 2 min. showed significantly reduced platelet adhesion with increasing ARC1779 concentrations; bars represent mean, error bars SEM, n=8. In FIG. 34B, after a 1 min. pre-perfusion period with untreated whole blood (normalized to 100%), platelets treated with 200 nM ARC1779 (triangles) failed to adhere to the collagen surface and already immobilized platelets. Untreated platelets (squares) increased the covered area 16-fold during 3 min. FIG. 34C illustrates platelet dwell time analysis with high temporal resolution. Human whole blood with labeled platelets was perfused over collagen at 1,500/s and the time of immobilization (dwell time) of 50 randomly chosen platelets was measured. 66% of platelets in 200 nM ARC1779 treated blood (triangles) were immobile for less than 1 s. after attaching to the surface and then detached, whereas 88% of untreated platelets (squares) did not detach during the observation period (p<0.001). The dark lines represent the mean. FIG. 34D shows the effect of ARC1779 in comparison with abciximab on platelet adhesion and thrombus formation following perfusion of untreated and treated human blood on injured porcine arterial surfaces in perfusion flow chambers at 6974/sec of shear rate. SEM of platelet adhesion.

FIGS. 35A-B show ARC1779 activity in platelet aggregation assays. FIG. 35A is a graph depicting the activity of ARC1779 in botrocetin-induced platelet aggregation (BIPA) (circles) and ADP-induced platelet aggregation (squares) of citrated human platelet rich plasma (PRP). Aptamer was added to pre-warmed PRP 1 minute prior to either botrocetin or ADP. Area under the curve (AUC) was measured for 6 minutes post addition of agonists. Percent inhibition was calculated from AUC. Data shown is from single representative donors. FIG. 35B is a graph depicting the inhibition of VWF-dependent platelet activation as assayed using a PFA-100® platelet function analyzer. Aperture occlusion time was measured (y-axis). In these experiments, curves were fit to the raw data and ˜IC₉₅ values estimated. Data shown averaged over all donors.

FIGS. 36A-B show the relationship of ARC1779 concentration to pharmacodynamic effects on platelet aggregation and template bleeding time. FIG. 36A is a graph depicting the aptamer concentration (y-axis)-time (x-axis) profile for ARC1779 dosed at 0.5 mg/kg in cynomolgus macaques (n=3). FIG. 36B is a graph depicting the PFA-100 closure time (y-axis) as a function of time (x-axis) for ARC1779 dosed at 0.5 mg/kg in cynomolgus macaques.

FIGS. 37A-B show aptamer efficacy in a model of occlusive thrombus formation. FIG. 37A is a bar graph depicting the average time to vessel occlusion (±SEM) in the electrical injury (EI) model of arterial thrombosis in cynomolgus macaques. The overall P value for the data set was <0.0001, while the P value for the comparison of the 700 nM ARC1779* and abciximab*groups with the saline group was <0.05 and the P value for the comparison of the 1300 μM ARC1779 (±SEM) and the saline group was <0.01. FIG. 37B is a bar graph depicting the average template bleeding time during the 6 hour infusion during the EI model (±SEM). The overall P value for the data set was <0.0001, while only the 1300 nM ARC1779* and abciximab*groups had statistically significant P values when compared with the saline group was (<0.001 in both cases).

### DETAILED DESCRIPTION

The details of one or more embodiments of the invention are set forth in the accompanying description below. Although any materials and methods similar or equivalent to those described herein can be used in the practice or testing of the present invention, the preferred materials and methods are now described. Other features, objects and advantages of the invention will be apparent from the description. In the description, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present description will control.

The present invention provides pharmaceutical compositions, or formulations, of aptamers that bind to von Willebrand Factor (vWF), referred to herein as “vWF aptamers”, and methods for using such vWF aptamers to treat vWF-mediated diseases and disorders, including the treatment of thrombotic disorders involving vWF-mediated platelet aggregation. The formulations comprise a vWF aptamer or a pharmaceutically acceptable salt thereof and a pharmaceutically acceptable solvent. The formulations and dosages described herein are designed to maximize clinical efficacy in the treatment of thrombotic disorders while simultaneously decreasing or minimizing adverse side effects, such as bleeding and/or complement activation.

The formulations described herein are stable. The term “stable”, as used herein, means remaining in a state or condition that is suitable for administration to a patient.

The formulations are, preferably, substantially pure. As used herein, “substantially pure” means the active ingredient (aptamer) is the predominant species present (i.e., on a molar basis it is more abundant than any other individual species in the composition), and preferably a substantially purified fraction is a composition wherein the active ingredient comprises at least about 50 percent (on a molar basis) of all macromolecular species present. Generally, a substantially pure composition will comprise more than 80% of all macromolecular species present in the composition, more preferably more than 85%, 90%, 95% and 99%. Most preferably, the active ingredient is purified to essential homogeneity (contaminant species cannot be detected in the composition by conventional detection methods) wherein the composition consists essentially of a single macromolecular species.

### vWF Aptamers

The formulations described herein comprise a vWF aptamer or a pharmaceutically acceptable salt thereof. The formulations may comprise any aptamer or a combination of aptamers that bind to vWF or a variant or a fragment thereof. Preferably, the aptamer binds full length vWF. If the aptamer binds a fragment of vWF, it is preferable that the aptamer bind the A1 domain of vWF. Preferably, the vWF aptamer specifically binds to a von Willebrand Factor full length target and a von Willebrand Factor domain A1 target, and more preferably, the vWF aptamer specifically binds to a human vWF full length target and a human vWF domain A1 target. The vWF protein may be from any species, but is preferably human. The vWF aptamer preferably comprises a dissociation constant for human von Willebrand Factor domain A1, or a variant thereof, of 100 nM or less, preferably 50 nM or less, preferably 10 nM or less, preferably 5 nM or less, preferably 1 nM or less, and more preferably 500 μM or less.

Aptamers and methods for identifying an aptamer that binds to vWF are known in the art. For example, U.S. patent application Ser. No. 11/222,346 and its corresponding applications describe such aptamers and methods in detail. For example, any of the following aptamers may be used in the formulations and methods of the invention: ARC1779, ARC1368, ARC1361, ARC1346, ARC1172, ARC1115 and ARC1029. Aptamers, including chemically substituted aptamers, can be synthesized using any oligonucleotide synthesis techniques known in the art, including solid phase oligonucleotide synthesis techniques (see, e.g., Froehler et al., Nucl. Acid Res., 14:5399-5467 (1986) and Froehler et al., Tet. Lett., 27:5575-5578 (1986)) and solution phase methods, such as triester synthesis methods (see, e.g., Sood et al., Nucl. Acid Res., 4:2557 (1977) and Hirose et al., Tet. Lett., 28:2449 (1978)).

The vWF aptamers used in the formulations and methods of the invention are ribonucleic acid, deoxyribonucleic acid or mixed ribonucleic acid and deoxyribonucleic acid. Aptamers may be single stranded ribonucleic acid, deoxyribonucleic acid or mixed ribonucleic acid and deoxyribonucleic acid. In some embodiments, the aptamer comprises at least one chemical modification. In some embodiments, the chemical modification is selected from a chemical substitution of the nucleic acid at a sugar position, a chemical substitution at a phosphate position and a chemical substitution at a base position. In other embodiments, the chemical modification is selected from incorporation of a modified nucleotide; 3′ capping; conjugation to a high molecular weight, non-immunogenic compound; conjugation to a lipophilic compound; and incorporation of phosphorothioate into the phosphate back bone. In a preferred embodiment, the non-immunogenic, high molecular weight compound is polyalkylene glycol, and more preferably is polyethylene glycol (PEG).

Those of ordinary skill in the art will appreciate that the composition of an oligonucleotide can influence complement activation. For example, the number of phosphorothioate substitutions is directly related to the relative degree of complement activation. Thus, in a preferred embodiment, the vWF aptamer used in the formulations and methods provided herein has a nucleotide sequence that includes no more than four, no more than three, no more than two or no more than one phosphorothioate backbone modification. Preferably, the binding affinity for vWF is increased relative to a second aptamer having the same nucleotide sequence but lacking the phosphorothioate backbone modification.

The vWF aptamer used in the formulations and methods described herein prevent vWF-mediated platelet aggregation, preferably while not significantly increasing bleeding time in a subject. That is, the vWF aptamer used in the formulations and methods described herein do not increase bleeding time in a subject in a clinically significant (i.e., clinically meaningful) manner. In some embodiments, an increase in bleeding time is less than 15 minutes, preferably less than 10 minutes, more preferably less than 5 minutes, and in some embodiments, less than 3 minutes relative to the bleeding time of a subject not treated with the aptamer. In some embodiments, the bleeding time is determined by cutaneous (or template) bleeding time. In addition, the vWF aptamer helps to rapidly restore platelet count to normal, near normal or functionally safe levels.

Preferably, the vWF aptamer binds to vWF or a variant or a fragment thereof and acts as an antagonist to inhibit the function of vWF. When vWF is activated, it is responsible for the adhesion, activation and aggregation of platelets, which are involved in the formation of blood clots.

Preferably, the vWF aptamer is ARC1779. ARC1779, is a synthetically manufactured, modified DNA/RNA aptamer that is conjugated to a polyethylene glycol (PEG, 20 kDa) moiety at the 5′-terminus.

ARC1779 is an aptamer having the following structure:

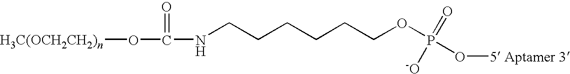

wherein: “n” is about 454 ethylene oxide units (PEG=20 kDa) and the aptamer comprises the following nucleic acid sequence or fragment thereof:
mGmCmGmUdGdCdAmGmUmGmCmCmUmUmCmGmGmCdCmG-s-dTmGdCdGdGdTmGmCdCmUdCdCmGmUdCmAmCmGmC-3T (SEQ ID NO: 1), wherein “m” is a 2′-OMe substituted nucleotide, “d” is a deoxyribonucleotide, “s” is a phosphorothioate internucleotide linkage and “3T” is an inverted deoxythymidine.

ARC1779 binds the A1 domain of human vWF and prevents the interaction of the vWF A1 domain with the platelet GPIb receptor. Therefore, ARC1779 is a competitive antagonist of the vWF/platelet interaction.

The core aptamer portion of ARC1779 (MW ˜13 kDa), is a 40-mer modified DNA/RNA oligonucleotide composed of 13 unmodified 2′-deoxyribonucleotides, 26 modified 2′-O-methyl-substituted nucleotides (to minimize endonuclease digestion), 1 inverted deoxythymidine nucleotide as a 3′ terminus “cap” (to minimize 3′ exonuclease digestion), and a single phosphorothioate linkage between nucleotide positions 20 (mG) and 21 (dT) (to enhance affinity for vWF). The core 40-mer is synthesized with a hexylamine at the 5′ terminus as a reactive site for subsequent conjugation (“PEGylation”) of a 20 kDa PEG moiety to form the active pharmaceutical ingredient ARC1779.

The chemical name for ARC1779 is:
N-(methoxy-polyethyleneglycol)-6-aminohexylyl-(1→5′)-2′-OMe-guanylyl-(3′→5′)-2′-OMe-cytidylyl-(3′→5′)-2′-OMe-guanylyl-(3′→5′)-2′-OMe-uracylyl-(3′→5′)-2′-deoxyguanylyl-(3′→5′)-2′-deoxycytidylyl-(3′→5′)-2′-deoxyadenylyl-(3′→5′)-2′-OMe-guanylyl-(3′→5′)-2′-OMe-uracylyl-(3′→5′)-2′-OMe-guanylyl-(3′→5′)-2′-OMe-cytidylyl-(3′→5′)-2′-OMe-cytidylyl-(3′→5′)-2′-OMe-uracylyl-(3′→5′)-2′-OMe-uracylyl-(3′→5′)-2′-OMe-cytidylyl-(3′→5′)-2′-OMe-guanyiyl-(3′→5′)-2′-OMe-guanylyl-(3′→5′)-2′-OMe-cytidylyl-(3′→5′)-2′-deoxycytidylyl-(3′→5′)-2′-OMe-P-thioguanylyl-(3′→5′)-2′-deoxythymidylyl-(3′→5′)-2′-OMe-guanylyl-(3′→5′)-2′-deoxycytidylyl-(3′→5′)-2′-deoxyguanylyl-(3′→5′)-2′-deoxyguanylyl-(3′→5′)-2′-deoxythymidylyl-(3′→5′)-2′-OMe-guanylyl-(3′→5′)-2′-OMe-cytidylyl-(3′→5′)-2′-deoxycytidylyl-(3′→5′)-2′-OMe-uracylyl-(3′→5′)-2′-deoxycytidylyl-(3′→5′)-2′-deoxycytidylyl-(3′→5′)-2′-OMe-guanylyl-(3′→5′)-2′-OMe-uracylyl-(3′→5′)-2′-deoxycytidylyl-(3′→5′)-2′-OMe-adenylyl-(3′→5′)-2′-OMe-cytidylyl-(3′→5′)-2′-OMe-guanylyl-(3′→5′)-2′-OMe-cytidylyl-(3′→5′)-(3′→3′)-2′-deoxythymidine.

The calculated molecular weight (MW) of ARC1779 is ˜33,138 Da. However, all doses and concentrations of ARC1779 are based on the core oligonucleotide mass, exclusive of the PEG mass. ARC1779 is soluble in water.

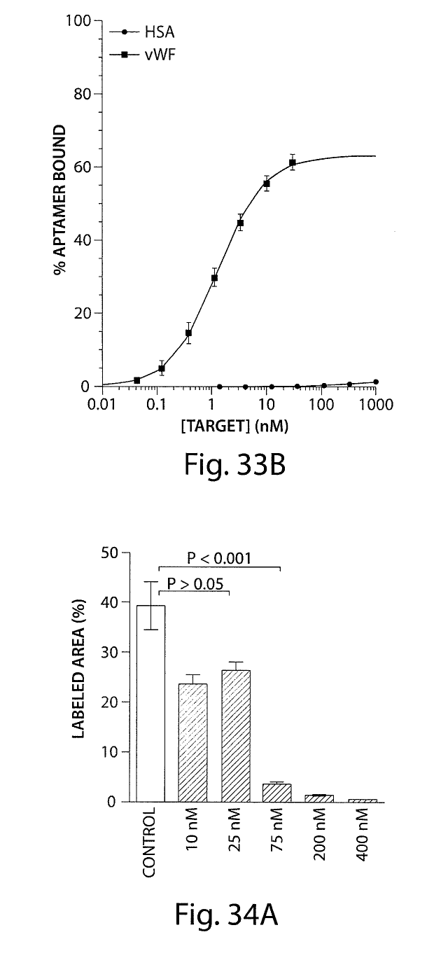

The proposed secondary structure of ARC1779 is depicted in FIG. 33A. ARC1779 binds to the A1 domain of human vWF with high affinity, preventing interaction with platelet GPIb. The core aptamer portion of ARC1779 (MW ˜13 kDa) is a 40-mer modified DNA/RNA oligonucleotide. The core 40-mer is synthesized with a hexylamine at the 5′-terminus as a reactive site for subsequent conjugation of a 20 kDa PEG moiety to form the active pharmaceutical ingredient ARC1779 (MW ˜33 kDa). As shown in FIG. 33B, the core oligonucleotide of ARC1779 binds to human vWF with high affinity (K_{D}˜2 nM).

The formulations described herein may use ARC1779 and/or other aptamers specifically capable of binding and modulating, e.g., antagonizing, full length von Willebrand Factor and/or von Willebrand Factor domain A1.

Examples of such other aptamers include, but are not limited to, ARC1368, ARC1361, ARC1346, ARC1172, ARC1115 and ARC1029.

ARC1368 is an aptamer having the following structure: mGmCmGmUdGdCdAmGmUmGmCmCmUmUmCmGmGmCdCmG-s-dTmGdCdGdGdTmGmCdCmUdCdCmGmUdCmAmCmGmC-3T (SEQ ID NO: 2), wherein the aptamer sequence is written in the 5′ to 3′ direction, “m” is a 2′-O Methyl substituted nucleotide, “d” is a deoxyribonucleotide, “s” is a phosphorothioate internucleotide linkage and “3T” is an inverted deoxythymidine.

ARC1361 is an aptamer having the following structure: mGmCmGmUdGdCdAmGmUmGmCmCmUmUmCmGmGmCdCmGdTmGdCdGdGdTmGm CdCmUdCdCmGmUdCmAmCmGmC-3T (SEQ ID NO: 3), wherein the aptamer sequence is written in the 5′ to 3′ direction, “m” is a 2′-O Methyl substituted nucleotide, “d” is a deoxyribonucleotide and “3T” is an inverted deoxythymidine.

ARC1346 is an aptamer having the following structure: mGmCmGmUdGdCdAmGmUmGmCmCmUmUmCmGmGmCdCmGdTmGdCdGdGdTmGm CdCmUdCdCmGmUmCmAmCmGmC-3T (SEQ ID NO: 4), wherein the aptamer sequence is written in the 5′ to 3′ direction, “m” is a 2′-O Methyl substituted nucleotide, “d” is a deoxyribonucleotide and “3T” is an inverted deoxythymidine.

ARC1172 is an aptamer having the following structure: dGdGdCdGdTdGdCdAdGdTdGdCdCdTdTdCdGdGdCdCdGdTdGdCdGdGdTdGdCdCdTdCd CdGdTdCdAdCdGdCdC-3T (SEQ ID NO: 5), wherein the aptamer sequence is written in the 5′ to 3′ direction, “d” is a deoxyribonucleotide and “3T” is an inverted deoxythymidine.

ARC1115 is an aptamer having the following structure: dGdGdCdGdTdGdCdAdGdTdGdCdCdTdTdCdGdGdCdCdGdTdGdCdGdGdTdGdCdCdTdCd CdGdTdCdAdCdGdCdC (SEQ ID NO: 6), wherein the aptamer sequence is written in the 5′ to 3′ direction and “d” is a deoxyribonucleotide.

ARC1029 is an aptamer having the following structure: GGCGTGCAGTGCC-[PEG]-GGCCGTGCGGTGCCTCCGTCACGCC-3T (SEQ ID NO: 7), wherein the sequence is written in the 5′ to 3′ direction, “3T” is an inverted deoxythymidine and “[PEG]” is a polyethylene glycol moiety.

As stated previously, the formulations may comprise a vWF aptamer or its pharmaceutically acceptable salt. As used herein, the term “pharmaceutically acceptable salt” refers to salt forms of the active compound that are prepared with counter ions that are non-toxic under the conditions of use and are compatible with a stable formulation. Examples of pharmaceutically acceptable salts of vWF aptamers include hydrochlorides, sulfates, phosphates, acetates, fumarates, maleates and tartrates.

The formulations may comprise any amount of vWF aptamer. Typically, the formulations comprise 1-50 mg/ml vWF aptamer or a pharmaceutically acceptable salt thereof. For example, suitable concentrations of vWF aptamer or a pharmaceutically acceptable salt thereof include, but are not limited to, 1-50 mg/ml or any 0.1 mg/ml increment thereof. Preferably, intravenous formulations comprise 5-25 mg/ml of vWF aptamer or a pharmaceutically acceptable salt thereof. More preferably, intravenous formulations comprise 5-15 mg/ml of vWF aptamer or a pharmaceutically acceptable salt thereof. Most preferably, intravenous formulations comprise 10 mg/ml of vWF aptamer or a pharmaceutically acceptable salt thereof. Alternatively, subcutaneous formulations preferably comprise 25-50 mg/ml of vWF aptamer or a pharmaceutically acceptable salt thereof. More preferably, subcutaneous formulations comprise 40-50 mg/ml of vWF aptamer or a pharmaceutically acceptable salt thereof. Most preferably, subcutaneous formulations preferably comprise 50 mg/ml of vWF aptamer or a pharmaceutically acceptable salt thereof. Preferably, subcutaneous formulations are low volume formulations.

### Pharmaceutically Acceptable Solvent

The formulations also comprise a pharmaceutically acceptable solvent. The term “pharmaceutically acceptable solvent”, as used herein, means being compatible with the other ingredients of the formulation and not deleterious to the recipient thereof. Pharmaceutically acceptable solvents are well known in the art. Examples of pharmaceutically acceptable solvents can be found, for example, in Goodman and Gillmans, The Pharmacological Basis of Therapeutics, latest edition. Preferably, the pharmaceutically acceptable solvent is selected from the group consisting of 0.9% saline (physiological saline or sterile isotonic saline solution) or phosphate buffered saline. Most preferably, the pharmaceutically acceptable solvent is 0.9% saline.

The formulations may comprise any amount of pharmaceutically acceptable solvent. Preferably, subcutaneous formulations comprise 0.1-2 ml of pharmaceutically acceptable solvent. For example, suitable subcutaneous formulations include, but are not limited to, 0.1-2 ml of pharmaceutically acceptable solvent or any 0.1 ml increment thereof. Most preferably, subcutaneous formulations comprise 1 ml or less of pharmaceutically acceptable solvent. Alternatively, intravenous formulations may comprise any amount of pharmaceutically acceptable solvent.

### Optional Components

The formulations of the invention only require a vWF aptamer or a pharmaceutically acceptable salt thereof, and a pharmaceutically acceptable solvent. However, various embodiments of the formulations may, optionally, include one or more of the following: buffer, pH adjuster, tonicity agent, cosolvent or pharmaceutically acceptable carrier.

In some embodiments, the formulations may further comprise a buffer. A buffer is any substance that, when added to a solution, is capable of neutralizing both acids and bases without appreciably changing acidity or alkalinity of the solution. Examples of buffers include, but are not limited to, pharmaceutically acceptable salts and acids of acetate, glutamate, citrate, tartrate, benzoate, lactate, histidine or other amino acids, gluconate, phosphate, malate, succinate, formate, propionate and carbonate.

In some embodiments, the formulations may further comprise a pH adjuster. A pH adjuster is used to adjust the pH of the formulation. Suitable pH adjusters typically include at least an acid or a salt thereof and/or a base or a salt thereof. Acids and bases can be added on an as needed basis in order to achieve a desired pH. For example, if the pH is greater than the desired pH, an acid may be used to lower the pH to the desired pH. Examples of acids include, but are not limited to, hydrochloric acid, phosphoric acid, citric acid, ascorbic acid, acetic acid, sulphuric acid, carbonic acid and nitric acid. By way of another example, if the pH is less than the desired pH, a base can be used to adjust the pH to the desired pH. Examples of bases include, but are not limited to, sodium hydroxide, potassium hydroxide, calcium hydroxide, sodium carbonate, sodium citrate, sodium acetate and magnesium hydroxide.

In some embodiments, the formulations may further comprise a tonicity agent. Tonicity agents are used to adjust the osmolality of the formulations in order to bring them closer to the osmotic pressure of body fluids, such as blood or plasma. Examples of tonicity agents include, but are not limited to, anhydrous or hydrous forms of sodium chloride, dextrose, sucrose, xylitol, fructose, glycerol, sorbitol, mannitol, potassium chloride, mannose, calcium chloride, magnesium chloride and other inorganic salts.

In some embodiments, the formulations may further comprise a cosolvent. A cosolvent is a solvent that is added to the aqueous formulation in a weight amount that is less than that of water and assists in the solubilization of the vWF aptamer. Examples of cosolvents include, but are not limited to, glycols, ethanol and polyhydric alcohols.

In some embodiments, the formulations may further comprise a pharmaceutically acceptable carrier. The term “pharmaceutically acceptable carrier”, as used herein, means being compatible with the other ingredients of the formulation and not deleterious to the recipient thereof. Pharmaceutically acceptable carriers are well known in the art. Examples of pharmaceutically acceptable carriers can be found, for example, in Goodman and Gillmans, The Pharmacological Basis of Therapeutics, latest edition.

### Dosing

The dose of vWF aptamer is administered so as to achieve a steady state blood concentration equal to at least the EC₉₀, and preferably of 2-12 μg/ml. The steady state blood concentration can be any concentration between, and including, 2 μg/ml and 12 μg/ml in increments of 0.1 μg/ml. Preferably, the dose achieves a steady state blood concentration of 3-μg/ml. More preferably, the dose achieves a steady state blood concentration of 3-8 μg/ml. Most preferably, the dose achieves a steady state blood concentration of 3-6 μg/ml.

The dose of vWF aptamer is administered in mg per kg (mg/kg) of body weight. The daily dose of vWF aptamer is 0.05-10 mg/kg. The daily dose can be any dose between, and including, 0.05 mg/kg and 10 mg/kg in increments of 0.01 mg/kg. For example, acceptable dosage ranges include, but are not limited to, 0.05-1 mg/kg, 0.5-1.5 mg/kg, 1-2 mg/kg, 1.5-2.5 mg/kg, 2-3 mg/kg, 2.5-3.5 mg/kg, 3-4 mg/kg, 3.5-4.5 mg/kg, 4-5 mg/kg, 4.5-5.5 mg/kg, 5-6 mg/kg, 5.5-6.5 mg/kg, 6-7 mg/kg, 6.5-7.5 mg/kg, 7-8 mg/kg, 7.5-8.5 mg/kg, 8-9 mg/kg, 8.5-9.5 mg/kg, 9-10 mg/kg and 9.5-10 mg/kg. This dosage may be administered in a single dose, multiple doses, as a continual dose or a combination thereof.

Preferably, in order to treat TMA/TPP, once the desired blood concentration is reached, a dose of vWF aptamer of 0.384-2.592 mg/kg is administered on a daily basis. More preferably, a dose of 0.576-2.16 mg/kg is administered on a daily basis. Even more preferably, a dose of 0.576-1.728 mg/kg is administered on a daily basis. Most preferably, a dose of 0.576-1.296 mg/kg is administered on a daily basis. This dosage may be administered in a single dose, multiple doses, as a continual dose or a combination thereof. For example, a loading dose or doses may be administered followed by a maintenance dose or doses. The exact dose, however, is determined by the physician and is dependent upon many factors, such as the age, weight, condition and response of the patient. To achieve the desired blood concentration level, any dose of 0.05-10 mg/kg can be used.

For example, for a body weight of 10-200 kg, the dosage level of vWF aptamer is 0.5-2000 mg per day. For an average body weight of 70 kg, the dosage level is 3.5-700 mg per day. The dose may be any one of 0.5, 1, 1.5, 2, 2.5, 3, 3.5, 4, 4.5, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000, 1005, 1010, 1015, 1020, 1025, 1030, 1035, 1040, 1045, 1050, 1055, 1060, 1065, 1070, 1075, 1080, 1085, 1090, 1095, 1100, 1105, 1110, 1115, 1120, 1125, 1130, 1135, 1140, 1145, 1150, 1155, 1160, 1165, 1170, 1175, 1180, 1185, 1190, 1195, 1200, 1205, 1210, 1215, 1220, 1225, 1230, 1235, 1240, 1245, 1250, 1255, 1260, 1265, 1270, 1275, 1280, 1285, 1290, 1295, 1300, 1305, 1310, 1315, 1320, 1325, 1330, 1335, 1340, 1345, 1350, 1355, 1360, 1365, 1370, 1375, 1380, 1385, 1390, 1395, 1400, 1405, 1410, 1415, 1420, 1425, 1430, 1435, 1440, 1445, 1450, 1455, 1460, 1465, 1470, 1475, 1480, 1485, 1490, 1495, 1500, 1505, 1510, 1515, 1520, 1525, 1530, 1535, 1540, 1545, 1550, 1555, 1560, 1565, 1570, 1575, 1580, 1585, 1590, 1595, 1600, 1605, 1610, 1615, 1620, 1625, 1630, 1635, 1640, 1645, 1650, 1655, 1660, 1665, 1670, 1675, 1680, 1685, 1690, 1695, 1700, 1705, 1710, 1715, 1720, 1725, 1730, 1735, 1740, 1745, 1750, 1755, 1760, 1765, 1770, 1775, 1780, 1785, 1790, 1795, 1800, 1805, 1810, 1815, 1820, 1825, 1830, 1835, 1840, 1845, 1850, 1855, 1860, 1865, 1870, 1875, 1880, 1885, 1890, 1895, 1900, 1905, 1910, 1915, 1920, 1925, 1930, 1935, 1940, 1945, 1950, 1955, 1960, 1965, 1970, 1975, 1980, 1985, 1990, 1995 and 2000 mg.

For example, for a body weight of 10-50 kg, the dosage level of vWF aptamer is 0.5-500 mg per day. The dose may be any one of 0.5, 1, 1.5, 2, 2.5, 3, 3.5, 4, 4.5, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495 and 500 mg.

For example, for a body weight of 50-100 kg, the dosage level of vWF aptamer is 2.5-1000 mg per day. The dose may be any one of 2.5, 3, 3.5, 4, 4.5, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995 and 1000 mg.

For example, for a body weight of 100-150 kg, the dosage level of vWF aptamer is 5-1500 mg per day. The dose may be any one of 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000, 1005, 1010, 1015, 1020, 1025, 1030, 1035, 1040, 1045, 1050, 1055, 1060, 1065, 1070, 1075, 1080, 1085, 1090, 1095, 1100, 1105, 1110, 1115, 1120, 1125, 1130, 1135, 1140, 1145, 1150, 1155, 1160, 1165, 1170, 1175, 1180, 1185, 1190, 1195, 1200, 1205, 1210, 1215, 1220, 1225, 1230, 1235, 1240, 1245, 1250, 1255, 1260, 1265, 1270, 1275, 1280, 1285, 1290, 1295, 1300, 1305, 1310, 1315, 1320, 1325, 1330, 1335, 1340, 1345, 1350, 1355, 1360, 1365, 1370, 1375, 1380, 1385, 1390, 1395, 1400, 1405, 1410, 1415, 1420, 1425, 1430, 1435, 1440, 1445, 1450, 1455, 1460, 1465, 1470, 1475, 1480, 1485, 1490, 1495 and 1500 mg.

For example, for a body weight of 150-200 kg, the dosage level of vWF aptamer is 7.5-2000 mg per day. The dose may be any one of 7.5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000, 1005, 1010, 1015, 1020, 1025, 1030, 1035, 1040, 1045, 1050, 1055, 1060, 1065, 1070, 1075, 1080, 1085, 1090, 1095, 1100, 1105, 1110, 1115, 1120, 1125, 1130, 1135, 1140, 1145, 1150, 1155, 1160, 1165, 1170, 1175, 1180, 1185, 1190, 1195, 1200, 1205, 1210, 1215, 1220, 1225, 1230, 1235, 1240, 1245, 1250, 1255, 1260, 1265, 1270, 1275, 1280, 1285, 1290, 1295, 1300, 1305, 1310, 1315, 1320, 1325, 1330, 1335, 1340, 1345, 1350, 1355, 1360, 1365, 1370, 1375, 1380, 1385, 1390, 1395, 1400, 1405, 1410, 1415, 1420, 1425, 1430, 1435, 1440, 1445, 1450, 1455, 1460, 1465, 1470, 1475, 1480, 1485, 1490, 1495, 1500, 1505, 1510, 1515, 1520, 1525, 1530, 1535, 1540, 1545, 1550, 1555, 1560, 1565, 1570, 1575, 1580, 1585, 1590, 1595, 1600, 1605, 1610, 1615, 1620, 1625, 1630, 1635, 1640, 1645, 1650, 1655, 1660, 1665, 1670, 1675, 1680, 1685, 1690, 1695, 1700, 1705, 1710, 1715, 1720, 1725, 1730, 1735, 1740, 1745, 1750, 1755, 1760, 1765, 1770, 1775, 1780, 1785, 1790, 1795, 1800, 1805, 1810, 1815, 1820, 1825, 1830, 1835, 1840, 1845, 1850, 1855, 1860, 1865, 1870, 1875, 1880, 1885, 1890, 1895, 1900, 1905, 1910, 1915, 1920, 1925, 1930, 1935, 1940, 1945, 1950, 1955, 1960, 1965, 1970, 1975, 1980, 1985, 1990, 1995 and 2000 mg.

Preferably, for a body weight of 10-200 kg, the dosage level of vWF aptamer is 3.84-518.4 mg per day. Preferably, for a body weight of 10-50 kg, the dosage level is 3.84-129.6 mg per day; for a body weight of 50-100 kg, the dosage level is 129.6-259.2 mg per day; for a body weight of 100-150 kg, the dosage level is 259.2-388.8 mg per day; and for a body weight of 150-200 kg, the dosage level is 388.8-518.4 mg per day. The dose may be any one of 3.84, 4, 4.5, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515 and 518.4 mg.

Preferably, for a body weight of 10-200 kg, the dosage level of vWF aptamer is 5.76-432 mg per day. Preferably, for a body weight of 10-50 kg, the dosage level is 5.76-108 mg per day; for a body weight of 50-100 kg, the dosage level is 108-216 mg per day; for a body weight of 100-150, the dosage level is 216-324 mg per day; and for a body weight of 150-200 kg, the dosage level is 324-432 mg per day. The dose may be any one of 5.76, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425 and 430 mg.

Preferably, for a body weight of 10-200 kg, the dosage level of vWF aptamer is 5.76-345.6 mg per day. Preferably, for a body weight of 10-50 kg, the dosage level is 5.76-86.4 mg per day; for a body weight of 50-100 kg, the dosage level is 86.4-172.8 mg per day; for a body weight of 100-150 kg, the dosage level is 172.8-259.2 mg per day; and for a body weight of 150-200 kg, the dosage level is 259.2-345.6 mg per day. The dose may be any one of 5.76, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345 and 345.6 mg.

Preferably, for a body weight of 10-200 kg, the dosage level of vWF aptamer is 5.76-259.2 mg per day. Preferably, for a body weight of 10-50 kg, the dosage level is 5.76-64.8 mg per day; for a body weight of 50-100 kg, the dosage level is 64.8-129.6 mg per day; for a body weight of 100-150 kg, the dosage level is 129.6-194.4 mg per day; and for a body weight of 150-200 kg, the dosage level is 194.4-259.2 mg per day. The dose may be any one of 5.76, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255 and 259.2 mg.

Preferably, in order to treat ACS, a dose or doses of vWF aptamer are administered sufficient to maintain the desired blood level concentration, preferably through the pre-intervention period, more preferably through the pre-intervention and intervention periods, and most preferably through the pre-intervention, intervention and initial post-intervention periods. Preferably, a dose of 0.3-10 mg/kg is administered.

### Administration

ARC1779 is manufactured for clinical use as a sterile isotonic saline solution (0.9% saline solution) for injection. Preferably, the formulation is provided in a 10 mg/mL solution. The formulation may be administered directly into an individual or may be diluted into an IV bag prior to administration.

The formulations are suitable for parenteral administration. Suitable routes for parenteral administration include intravenous, subcutaneous, intradermal, intramuscular, intraarticular and intrathecal administration. Suitable routes of administration may also be used in combination, such as intravenous administration followed by subcutaneous administration. The route of administration, however, is determined by the attending physician. Preferably, the formulations are administered subcutaneously. Most preferably, the formulations are administered intravenously.

The formulations may be administered parenterally, for example, as a bolus; a slow bolus over a short period of time, such as 15 minutes; a continual infusion or a continual drip. Preferably, the formulations are administered by continual infusion.

Administration by continual infusion may be at a constant rate. Alternatively, the rate of administration may be varied (not constant) over time in order to take into account a loading doses prior to or at the beginning of administration and tapering of the infusion rate at the end of administration. Preferably, the rate of continual infusion is varied.

### Preferred Dosing and Administration

Preferably, in order to treat TMA/TPP, the formulations are administered using a loading dose, a maintenance dose and a tapered dose.

Preferably, the loading dose is an intravenous infusion of vWF aptamer in a dose- and duration-escalation design. For example, the loading dose may be administered in three ten-minute intervals over 30 minutes or three twenty-minute intervals over 60 minutes. In the first ten-minute or twenty-minute interval, a dose of X mg/kg is administered by constant infusion. In the second ten-minute or twenty-minute interval, a dose of 2×mg/kg is administered by constant infusion. In the third ten-minute or twenty-minute interval, a dose of 4×mg/kg is administered by constant infusion. The sum of X, 2× and 4× equals the total loading dose. Preferably, the total loading dose is 0.093-0.56 mg/kg. More preferably, the total loading dose is 0.14-0.466 mg/kg. Even more preferably, the total loading dose is 0.14-0.373 mg/kg. Most preferably, the total loading dose is 0.14-0.28 mg/kg. However, the dose and duration may be varied to achieve essentially the same result of a safe and tolerable dose that rapidly achieves the desired steady state concentration.

Preferably, the maintenance dose of vWF aptamer is administered as a continual infusion at a constant rate. Preferably, the maintenance dose is administered at a rate of 0.0002-0.0018 mg/kg/min, which is equal to 0.384-2.592 mg/kg/day, respectively. More preferably, the maintenance dose is administered at a rate of 0.0004-0.0015 mg/kg/min, which is equal to 0.576-2.16 mg/kg/day, respectively. Even more preferably, the maintenance dose is administered at a rate of 0.0004-0.0012 mg/kg/min, which is equal to 0.576-1.728 mg/kg/day, respectively. Most preferably, the maintenance dose is administered at a rate of 0.0004-0.0009 mg/kg/min, which is equal to 0.576-1.296 mg/kg/day, respectively. Preferably, the maintenance dose is administered as a continuous infusion until normalization of platelet count is achieved. Normalization is defined as a platelet count ≧˜150×10⁹ per liter measured over three consecutive days.

Preferably, the maintenance dose of vWF aptamer is tapered by 50% (½ of the administration rate) on the next to last day of administration and by another 50% (½ of the administration rate to 25% of the total) on the last day of administration. The infusion will be stopped after the tapering procedure.

Alternatively, the formulation may be administered as the loading dose described above (dose- and duration-escalation design) followed by a subcutaneous injection.

In another embodiment and after plasma exchange, half of the original loading dose of vWF aptamer is administered over 30 minutes. Preferably, the formulation is administered 0-30 minutes, and more preferably 0-15 minutes, after plasma exchange. The purpose of this is to restore aptamer concentration to the target level. Then, the continuous infusion will resume.

In specific embodiments, the formulations are dosed and administered according to the following Table 0.

TABLE 0

| | Target | 2 | 3 | 6 | 8 | 10 | 12 |
|---|---|---|---|---|---|---|---|
| | [plasma] | μg/ml | μg/ml | μg/ml | μg/ml | μg/ml | μg/ml |
| | Total loading | 0.093 | 0.14 | 0.28 | 0.373 | 0.466 | 0.56 |
| | dose | mg/kg | mg/kg | mg/kg | mg/kg | mg/kg | mg/kg |
| Infusion regimen | loading dose | 0.0133 | 0.02 | 0.04 | 0.0533 | 0.0666 | 0.08 |
| (mg/kg) | 0-10 min or | mg/kg | mg/kg | mg/kg | mg/kg | mg/kg | mg/kg |
| | 0-20 min | | | | | | |
| Infusion regimen | loading dose | 0.0266 | 0.04 | 0.08 | 0.1066 | 0.133 | 0.16 |
| (mg/kg) | 11-20 min or | mg/kg | mg/kg | mg/kg | mg/kg | mg/kg | mg/kg |
| | 21-40 min | | | | | | |
| Infusion regimen | loading dose | 0.053 | 0.08 | 0.16 | 0.213 | 0.266 | 0.32 |
| (mg/kg) | 21-30 min or | mg/kg | mg/kg | mg/kg | mg/kg | mg/kg | mg/kg |
| | 41-60 min | | | | | | |
| Infusion regimen | loading dose | 0.00132 | 0.002 | 0.004 | 0.00532 | 0.0066 | 0.008 |
| (mg/kg/min) | 0-10 min | | | | | | |
| Infusion regimen | loading dose | 0.00066 | 0.001 | 0.002 | 0.00266 | 0.0033 | 0.004 |
| (mg/kg/min) | 0-20 min | | | | | | |
| Infusion regimen | loading dose | 0.00266 | 0.004 | 0.008 | 0.0106 | 0.0132 | 0.016 |
| (mg/kg/min | 11-20 min | | | | | | |
| Infusion regimen | loading dose | 0.00133 | 0.002 | 0.004 | 0.0053 | 0.0066 | 0.008 |
| (mg/kg/min) | 21-40 min | | | | | | |
| Infusion regimen | loading dose | 0.00532 | 0.008 | 0.016 | 0.0212 | 0.0198 | 0.32 |
| (mg/kg/min) | 21-30 | | | | | | |
| Infusion regimen | loading dose | 0.00266 | 0.004 | 0.008 | 0.0106 | 0.0099 | 0.16 |
| (mg/kg/min) | 41-60 min | | | | | | |
| | Maintenance | 0.384 | 0.576 | 1.296 | 1.728 | 2.16 | 2.592 |
| | dose | | | | | | |
| | (mg/kg/day) | | | | | | |
| Infusion regimen | Maintenance | 0.000266 | 0.0004 | 0.0009 | 0.0012 | 0.0015 | 0.0018 |
| (mg/kg/min) | dose | | | | | | |

More preferably, in order to treat ACS, a dose of 0.3 mg/kg is administered. Even more preferably, a dose of 0.6 mg/kg is administered. Most preferably, a dose of 1.0 mg/kg is administered. Preferably, the formulation is administered as a bolus or a slow bolus over a 15 minute time period.

The formulations may be administered to a vertebrate, preferably a mammal, and more preferably a human. The terms “patient” and “subject” are used interchangeably throughout the application, and these terms include both human and veterinary subjects. The aptamer formulations provided herein are administered to subjects, particularly, human subjects, in an amount effective to inhibit, reduce, block or otherwise modulate vWF-mediated platelet aggregation.

### Indications

The formulations are used to treat, prevent or ameliorate vWF-mediated diseases and disorders, including the treatment of thrombotic disorders involving vWF-mediated platelet aggregation. The diseases and disorders to be treated, prevented or ameliorated are selected from the group consisting of: essential thrombocytopenia, thrombotic microangiopathies (TMA), thrombotic thrombocytopenic purpura (TTP), Type 2b von Willebrand's Disease, pseudo type 2b von Willebrand's Disease, peripheral artery disease, e.g., peripheral arterial occlusive disease, unstable angina, angina pectoris, arterial thrombosis, atherosclerosis, myocardial infarction, acute coronary syndrome (ACS), atrial fibrillation, carotid stenosis, unstable carotid lesions, cerebral infarction, cerebral thrombosis, ischemic stroke, and transient cerebral ischemic attack. In some unstable carotid disease embodiments, the pharmaceutical composition of the invention is administered prior to, during and/or after carotid revascularization procedures, either percutaneously or surgically.

### Therapeutic Rationale

Without wishing to be bound by theory regarding mechanism of action, the following therapeutic rationale is offered by way of example only.

### ACS

Because activated vWF plays a role in thrombus formation, the anti-vWF aptamer formulations provided herein are used to inhibit activated vWF in order to improve the outcome for ACS patients. The aptamer formulations provided herein address significant, unmet medical needs in the treatment of patients who are suffering from ACS, or heart attack, and who are undergoing a procedure called angioplasty, or PCI. These unmet needs include the improvement of blood flow to the heart, reduction of bleeding risk and improved therapeutic administration.

There is a large surface area of interaction between the A1 domain and its platelet binding site. An aptamer, with its large, three dimensional structure, is better suited to block this interaction than small molecules, which may be too small to effectively block an interaction between proteins with a large surface area. In addition, while biologics, such as monoclonal antibodies, with their large size, may be well-suited to block this interaction, it is difficult to rationally design the duration of the action of an antibody for an acute care procedure such as PCI. In one embodiment, the duration of the anti-platelet function following slow bolus infusion over 15 minutes is approximately equal to the length of the PCI procedure, thereby allowing reversal of the vWF inhibition following the completion of the procedure. The aptamer referred to herein as ARC1779 has this duration of action.

ARC1779 binds with high affinity and specificity to a region of activated vWF known as the A1 domain. When exposed to high shear forces, the A1 domain binds to its receptor on a platelet. Once bound, the platelet adheres to the blood vessel wall and then recruits and activates additional platelets. As these platelets aggregate, a thrombus is formed. The aptamers provided herein are agents that inhibit the three steps of platelet activity—adhesion, activation and aggregation—and, therefore, participate in all aspects of platelet-mediated thrombosis in order to provide a more precise and effective method for preventing platelet-induced thrombus formation than currently approved drugs.

By targeting vWF, ARC1779 improves myocardial perfusion in patients suffering from ACS. ARC1779 inhibits the local activation of vWF and prevents clot formation in the microvasculature. By targeting vWF, ARC1779 also reduces bleeding risk during PCI. Because ARC1779 targets and binds to only activated vWF, the anti-platelet effect of ARC1779 should only be present in regions subject to high shear forces. These shear forces are only present in the arteries, including those leading into and within the heart. These shear forces would be especially high in areas of vessels partially occluded by the presence of atherosclerotic lesions. In these areas, it has been estimated that shear force can be as high as 40 times that of the normal artery. Therefore, ARC1779 can locally suppress platelet function and thrombus formation in the coronary arteries, while not disrupting normal platelet function and blood clotting in the remainder of the body.

The vWF aptamer formulations, e.g., ARC1779 formulations, are used in the treatment of ACS patients who are undergoing percutaneous coronary intervention (PCI). The formulations and dosages are designed to improve the risk-to-benefit profile of adjuvant pharmacotherapy of PCI for high risk patients by introducing a novel anti-thrombotic therapeutic principle, namely antagonism of the binding of vWF to the GPIb receptor on platelets. vWF antagonism, using the aptamer formulations provided herein, provides anti-thrombotic efficacy comparable to GPIIb/IIIa antagonism, while reducing the relative risk of bleeding complications. In addition, the vWF aptamers help restore platelet count to within the normal range.

### TAM/TTP

Thrombotic microangiopathies (TMAs) are a result of impaired cleavage of vWF. This is due to a deficiency or defect of the vWF protease, ADAMTS-13, which leads to the formation of ultra-large vWF multimers that act as nidus for platelet-rich thrombi. The rationale for blocking vWF is to inhibit the formation of diffuse microvascular thrombi and improve end-organ perfusion, thereby reducing ischemia.

Using the aptamer formulations provided herein, e.g., ARC1779 formulations, to target activated vWF reduces or eliminates the formation of blood clots that cause the morbidity and mortality associated with TTP.

vWD

von Willebrands Disease (vWD) is also characterized by vWF abnormalities. For example, type 2 vWD is characterized by defective vWF. The rationale for treating vWD-type 2b is to reduce thrombocytopenia and enable the use of concomitant procoagulant therapy.

### Atherothrombosis

Atherothrombosis is a result of elevated and activated vWF. This is due to the fact that endothelial injury and shear forces in atherosclerotic arteries lead to vWF secretion and activation, which promotes platelet adhesion, activation and aggregation. The rationale for blocking vWF is to inhibit shear-dependent arterial thrombosis and improve end-organ perfusion, thereby reducing ischemia.

### Combination Therapy

One embodiment of the invention comprises a formulation of the invention used in combination with one or more other treatments for thrombotic related disorders. The aptamer formulation of the invention may contain, for example, more than one aptamer, e.g., an anti-thrombin aptamer and an anti-vWF aptamer. In some embodiments, an aptamer formulation of the invention, containing one or more aptamers, is administered in combination with another useful formulation or drug, such as an anti-inflammatory agent, an immunosuppressant, an antiviral agent, or the like. In another embodiment, the aptamer formulations are used in combination with non-drug therapies or treatments, such as plasma exchange and PCI. In general, the currently available dosage forms of the known therapeutic agents and the uses of non-drug therapies for use in such combinations will be suitable.

“Combination therapy” (or “co-therapy”) includes the administration of an aptamer formulation of the invention and at least a second agent or treatment as part of a specific treatment regimen intended to provide the beneficial effect from the co-action of these therapeutic agents or treatments. The beneficial effect of the combination includes, but is not limited to, pharmacokinetic or pharmacodynamic co-action resulting from the combination of therapeutic agent or treatments. Administration of these therapeutic agents or treatments in combination typically is carried out over a defined time period (usually minutes, hours, days or weeks depending upon the combination selected).

“Combination therapy” may, but generally is not, intended to encompass the administration of two or more of these therapeutic agents or treatments as part of separate monotherapy regimens that incidentally and arbitrarily results in the combinations of the present invention. “Combination therapy” is intended to embrace administration of these therapeutic agents or treatments in a sequential manner, that is, wherein each therapeutic agent or treatment is administered at a different time, as well as administration of these therapeutic agents or treatments, or at least two of the therapeutic agents or treatments, in a substantially simultaneous manner. Substantially simultaneous administration can be accomplished, for example, by administering to the subject a single injection having a fixed ratio of each therapeutic agent or in multiple, single injections for each of the therapeutic agents.

Sequential or substantially simultaneous administration of each therapeutic agent or treatment can be effected by any appropriate route including, but not limited to, topical routes, oral routes, intravenous routes, subcutaneous, intramuscular routes, and direct absorption through mucous membrane tissues. The therapeutic agents or treatments can be administered by the same route or by different routes. For example, a first therapeutic agent or treatment of the combination selected may be administered by injection while the other therapeutic agents or treatments of the combination may be administered subcutaneously. Alternatively, for example, all therapeutic agents or treatments may be administered subcutaneously or all therapeutic agents or treatments may be administered by injection. The sequence in which the therapeutic agents or treatments are administered is not critical unless noted otherwise.

“Combination therapy” also can embrace the administration of the therapeutic agent or treatments as described above in further combination with other biologically active ingredients. Where the combination therapy comprises a non-drug treatment, the non-drug treatment may be conducted at any suitable time so long as a beneficial effect from the co-action of the combination of the therapeutic agent and non-drug treatment is achieved. For example, in appropriate cases, the beneficial effect is still achieved when the non-drug treatment is temporally removed from the administration of the therapeutic agent, perhaps by days or even weeks.

The formulations may be administered in combination with other drugs or therapies. For example, the formulations of the invention may be used in combination with plasma exchange, corticosteroids, immunosuppressives, aspirin, clopidogrel, or aspirin and clopidogrel for use in treating TMAs. By way of another example, the formulations may be administered in combination with aspirin, clopidogrel, or aspirin and clopidogrel for use in treating ACS and TMAs. As a further example, the formulations may be administered in combination with antibiotics for use in treating HUS. The formulations are also compatible with other standard hypersensitivity regimens, such as corticosteroids and antihistamines.

### Packaging

The formulations can be packaged for use in a variety of pharmaceutically acceptable containers using any pharmaceutically acceptable container closure, as the formulations are compatible with PVC-containing and PVC-free containers and container closures. Examples of pharmaceutically acceptable containers include, but are not limited to, ampuls, pre-filled syringes, intravenous bags, intravenous bottles and admix bags. For example, the formulation may be an aqueous formulation containing both vWF aptamer and pharmaceutically acceptable solvent. Alternatively, the formulation may contain lyophilized vWF aptamer in one compartment of an admix bag and a pharmaceutically acceptable solvent in a separate compartment of the admix bag such that the two compartments may be mixed together prior to administration to a patient. Pharmaceutically acceptable containers are well known in the art and commercially available. Preferably, the formulations are stored in a Type 1 glass vial with a butyl rubber stopper. The formulations in liquid form must be stored in a refrigerated environment. Preferably, the liquid formulations are stored at 4° C. Alternatively, the lyophililized formulations may be stored at room temperature, or refrigerated or frozen.

Preferably, the formulations are sterile. A “sterile” formulation, as used herein, means a formulation that has been brought to a state of sterility and has not been subsequently exposed to microbiological contamination, i.e., the container holding the sterile composition has not been compromised. Sterile compositions are generally prepared by pharmaceutical manufacturers in accordance with current Good Manufacturing Practice (“cGMP”) regulations of the U.S. Food and Drug Administration.

Procedures for filling pharmaceutical formulations in pharmaceutically acceptable containers, and their subsequent processing are known in the art. These procedures can be used to produce sterile pharmaceutical drug products often required for health care. See, e.g., Center for Drug Evaluation and Research (CDER) and Center for Veterinary Medicine (CVM), “Guidance for Industry for the Submission Documentation for Sterilization Process Validation in Applications for Human and Veterinary Drug Products”, (November 1994). Examples of suitable procedures for producing sterile pharmaceutical drug products include, but are not limited to, terminal moist heat sterilization, ethylene oxide, radiation (i.e., gamma and electron beam) and aseptic processing techniques. Any one of these sterilization procedures can be used to produce the sterile pharmaceutical formulations described herein.

In some embodiments, sterile pharmaceutical formulations can be prepared using aseptic processing techniques. Sterility is maintained by using sterile materials and a controlled working environment. All containers and apparatus are sterilized, preferably by heat sterilization, prior to filling. Then, the container is filled under aseptic conditions, such as by passing the composition through a filter and filling the units. Therefore, the formulations can be sterile filled into a container to avoid the heat stress of terminal sterilization.

In some embodiments, the formulations are terminally sterilized using moist heat. Terminal sterilization can be used to destroy all viable microorganisms within the final, sealed container containing the pharmaceutical formulation. An autoclave is typically used to accomplish terminal heat-sterilization of drug products in their final packaging. Typical autoclave cycles in the pharmaceutical industry to achieve terminal sterilization of the final product are 121° C. for at least 10 minutes.

### Kits

The formulations may also be packaged in a kit. The kit will contain the formulation, along with instructions regarding administration of the drug. The kit may also contain one or more of the following: a syringe, an intravenous bag or bottle, the same drug in a different dosage form or another drug. For example, the kit may contain both an intravenous formulation and a subcutaneous formulation of the present invention. Alternatively, the kit may contain lyophilized vWF aptamer and an intravenous bag of solution. The kit form is particularly advantageous when the separate components must be administered in different dosage forms (i.e., parenteral and oral) or are administered at different dosage intervals.

Preferably, the kits are stored at 5±3° C. The kits can also be stored at room temperature or frozen at −20° C.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

### EXAMPLES

The following examples, including the experiments conducted and results achieved are provided for illustrative purposes only and are not to be construed as limiting upon the present invention.

For all of the following examples, ARC1779 was provided in a formulation comprising ARC1779 and isotonic saline.

All concentrations and doses of ARC1779 are expressed on the basis of oligonucleotide mass, exclusive of PEG mass.

### Example 1

### Pharmacokinetics and Pharmacodynamics of an Aptameric Antagonist of von Willebrand Factor

ARC1779 was investigated in a Phase 1 clinical trial conducted in healthy volunteers. This first-in-human study evaluated five ascending doses of ARC1779 from 0.05 to 1.0 mg/kg, each administered as a single IV bolus, and one dose of ARC1779 administered as an initial 0.3 mg/kg bolus followed by 4-hour infusion at 0.00125 mg/kg/min. Pharmacokinetic (PK) and pharmacodynamic (PD) findings of this study are described herein.

Blood samples were collected for analysis of PD effects of ARC1779, effects on platelet aggregation and determination of plasma concentrations of ARC1779. ARC1779 was given to subjects according to a weight-adjusted dosing regimen.

This study was an ascending-dose, double-blind, placebo-controlled study in 47 healthy volunteers at doses of 0 (placebo, n=6) or 0.05 to 1.0 mg/kg ARC1779 (n=41) given via IV push, “slow bolus” IV infusion over 15 minutes, or “slow bolus” followed by 4-hour IV infusion. PK parameters were estimated from plasma ARC1779 concentrations determined with a validated assay. PD effects were measured by an ELISA for free vWF A1 binding sites and by a platelet function analyzer, the PFA-100®.

Part A of the study assessed the PK and PD relative to placebo of 5 ascending dose levels of ARC1779 (0.05, 0.1, 0.3, 0.6, or 1.0 mg/kg) each administered as a single, IV bolus. After completion of the first 2 cohorts (6 subjects per cohort) (0.05, 0.1 mg/kg) and dosing of 5 subjects enrolled in Cohort 3 (0.3 mg/kg), the method of bolus administration was modified from IV push to administration as a short infusion or “slow bolus” over a period of 15 minutes. Additional subjects were then enrolled at the 0.1 and 0.3 mg/kg dose levels to allow evaluation of ARC1779 administration at these dose levels by slow bolus. As a result of these changes, Part A of the study enrolled 41 subjects (7 cohorts, N=6/cohort, with exception of Cohort 3, N=5, see Table 1). Within each dose level of Part A, subjects were randomized to ARC1779 or placebo at a ratio of 5:1. To minimize risk to study subjects, safety and tolerability data (collected up to and including Day 2) were reviewed at each dose level prior to escalation to the next dose level.

Part B of the study assessed the PK and PD relative to placebo of a single dose level of ARC1779 administered as a single, IV slow bolus over 15 minutes followed by continuous IV infusion for 4 hours. Part B enrolled a single cohort of 6 subjects randomized (5:1) to receive either ARC1779 or placebo. ARC1779 was given to subjects in Part B as an initial slow bolus of 0.3 mg/kg over 15 minutes followed by infusion of an additional 0.3 mg/kg over 4 hours at a rate of 0.00125 mg/kg/min.

A total of 47 healthy subjects were enrolled in the study and were dosed with ARC1779. Of this total, 41 subjects (Cohorts 1-7) participated in the dose-escalation arm (Part A) and 6 subjects (Cohort 8) participated in the bolus plus infusion arm (Part B).

At this time, PK and PD data are available for the 41 subjects of Part A: 6 subjects who received placebo and 35 subjects who received ARC1779 via either IV push or slow bolus administration (Table 1).

TABLE 1
Dosing Cohorts for Study Part A (N = 41)

| Cohort | Dose Level (mg/kg) | Administration Method | N |
|---|---|---|---|
| 1 | 0.05 | IV push | 6 |
| 2 | 0.1 | IV push | 6 |
| 3 | 0.3 | IV push | 5 |
| 4 | 0.1 | Slow bolus | 6 |
| 5 | 0.3 | Slow bolus | 6 |
| 6 | 0.6 | Slow bolus | 6 |
| 7 | 1.0 | Slow bolus | 6 |

### Patient Disposition and Demographics

In Part A of the study, six (6) subjects received a single dose of placebo (0.9% saline, USP) and 35 subjects received a single dose of ARC1779 at 0.05, 0.1, 0.3, 0.6 or 1.0 mg/kg by either IV push or slow bolus administration. Overall, the placebo and active treatment (ARC1779) groups were similar in terms of demographic characteristics.

Of the 35 subjects in the active treatment group of Part A, 34 (˜97%) were male and 1 (˜3%) was female. Twenty-five (˜71%) of the subjects were Black or African American, 9 (˜26%) of the subjects were White and 1 (˜3%) subject was Asian. Two subjects (˜6%) were of Latino or Hispanic ethnicity, while the majority (33, ˜94%) were identified as Not Hispanic or Latino.

The mean age of subjects in the active treatment group was 33 (±9.7) years, with a median age of 31 years, and a range from 20 to 55 years. The mean weight of subjects was 81.9 (±12.6) kg, with a median weight of 83.9 kg, and a range from 58.3 to 103.6 kg. The mean height of subjects was 175.7 (±6.5) cm, with a median height of 178 cm, and a range from 163 to 188 cm. The mean body mass index (BMI) of subjects was 26.5 (±3.4 kg/m²), with a median BMI of 26.4 and a range from 19.4 to 33.1 kg/m².

Blood-group typing classified 13 (˜38%) of the 35 subjects in the active treatment group to blood group A, 12 (˜35%) to blood group O, 5 (115%) to blood group B and 4 (˜12%) to blood group AB. Blood group typing information was not available for 1 of the 35 subjects.

In part B of the study, one subject received a single dose of placebo (0.9% saline, USP) and 5 subjects received a single dose of ARC1779 at 0.3 mg/kg by IV slow bolus administration followed by continuous infusion.

Of the 5 subjects in the active treatment group of Part B, 5 (100%) were male. Four (80%) of the subjects were Black or African American and 1 (20%) of the subjects was White.

The mean age of subjects in the active treatment group was 34 (±6.9) years, with a range from 25 to 42 years. The mean weight of subjects was 74.4 (±5.7) kg, with a range from 68.3 to 78.9 kg. The mean height of subjects was 173.4 (±3.6) cm, with a range from 168 to 178 cm. The mean body mass index (BMI) of subjects was 24.7 (±1.8 kg/m²), with a range from 22.8 to 26.4 kg/m².

Blood-group typing classified 2 (40%) of the 5 subjects in the active treatment group to blood group A, 3 (60%) to blood group O, 0 (0%) to blood group B and 0 (0%) to blood group AB.

### Other Clinical Assessments

The following clinical assessments were measured: vital signs, such as heart rate, respiratory rate, blood pressure and temperature; ECG; a physical exam that included height and weight, evaluation of general appearance, mental status, HEENT, and the following body systems: dermatologic, cardiovascular, respiratory, gastrointestinal, extremities and neurological; clinical laboratory assessments, such as clinical chemistry and hematology parameters; cutaneous bleeding time and complement activation.

### CBT

Cutaneous bleeding time (CBT) was measured as a proxy for bleeding risk potentially associated with ARC1779 administration. CBT was determined by the standard template method with a maximal period of observation of 20 minutes. Samples for CBT measurements were collected at Screening (subjects with CBT>15 minutes were excluded from the study), Day 0 prior to drug administration and Day 0 post-drug administration (at ˜15, 240 and 1440 minutes).

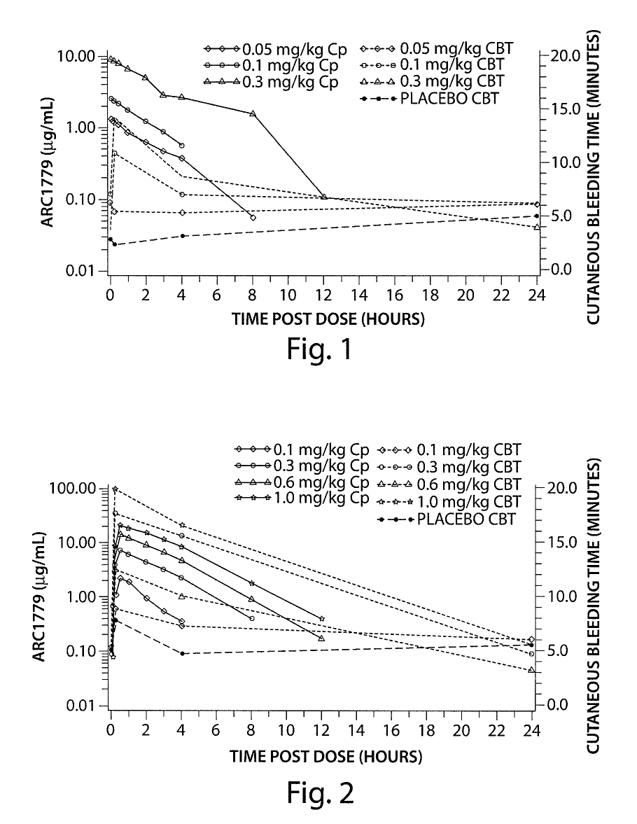

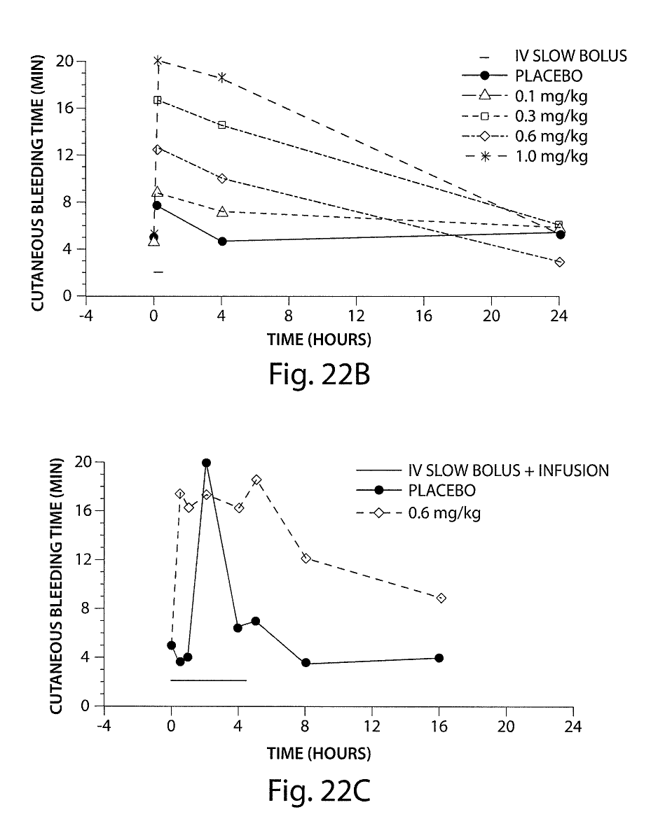

There was a high degree of variability in CBT values within each cohort and in subjects treated with placebo. CBT values as high as 13 minutes were observed “post-dose”. Mean CBT values over time for the 0.05, 0.1 and 0.3 mg/kg IV push dose groups (Cohorts 1-3), and the 0.1, 0.3, 0.6 and 1.0 mg/kg slow bolus dose groups (Cohorts 4-7) are shown in FIG. 1 and FIG. 2, respectively, and in FIG. 22. Dose-related increases in CBT (≦20 minutes) relative to placebo were observed at 15 and 240 minutes (4 hours) post-dose in IV push and slow bolus dose groups; however, these increases were modest and CBT returned to baseline by 1440 minutes post-dose (24 hours) in all groups. ARC1779 administration was not associated with sustained, clinically significant prolongation of CBT at the single, IV bolus doses evaluated. The maximal increase in CBT to ˜19-20 minutes occurred during the initial 4 hours post-dose at the highest (1.0 mg/kg) dose level with mean ARC1779 plasma concentrations over that interval of ˜8-9 μg/mL.

### Complement Activation

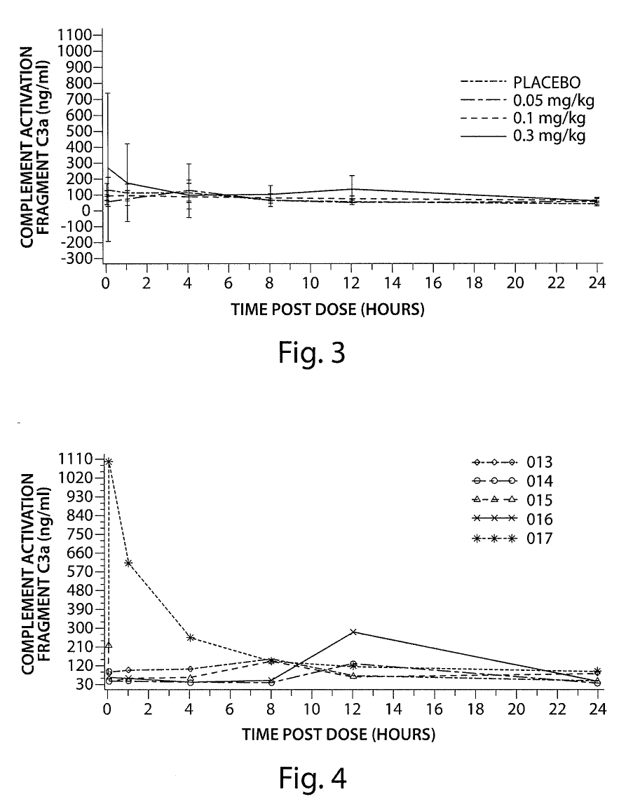

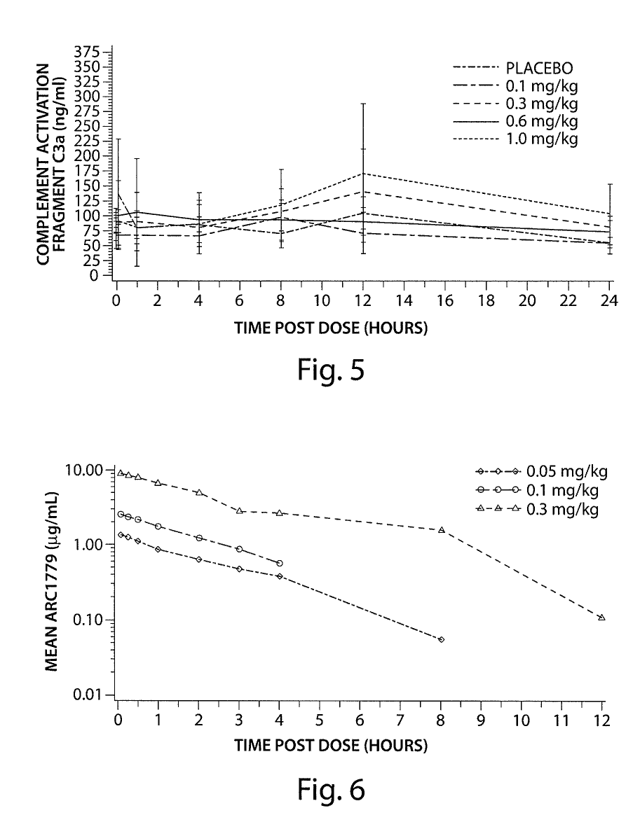

Blood samples were collected during Part A of the study in order to determine levels of complement protein fragment C3a as a measure of complement system activation. Samples for complement analysis were collected at Day 0 prior to drug administration and on Day 0 post-drug administration (at ˜5, 60, 240, 480, 720 and 1440 minutes). Mean C3a concentration data for subjects in the 0.05, 0.1 and 0.3 mg/kg IV push dose groups (Cohorts 1-3), and the 0.01, 0.3, 0.6 and 1.0 mg/kg slow bolus dose groups (Cohorts 4-7) are shown in FIG. 3 and FIG. 5, respectively. C3a concentration data for individual subjects in the 0.3 mg/kg IV push dose group (Cohort 3) are shown in FIG. 4.

No clinically significant alterations in C3a levels were observed up to 1440 minutes (24 hours) post-dose in subjects who received ARC1779 by IV push administration at the 0.05 and 0.1 mg/kg dose levels (Cohorts 1 and 2), or among subjects in subsequent cohorts (Cohorts 4-7) to whom ARC1779 was administered by slow bolus infusion at dose levels from 0.1 mg/kg to 1.0 mg/kg. As a group, subjects at the 0.3 mg/kg dose level to whom ARC1779 was administered by the IV push method (Cohort 3) showed a modest elevation in mean C3a concentration post-dose relative to subjects who received placebo (FIG. 3). However, this increase could be attributed largely to a single subject (Subject 017) in Cohort 3 who experienced a hypersensitivity reaction in association with rapid administration of ARC1779 by the IV push method, and showed significant elevations (up to ˜18-fold) in concentrations of complement fragment C3a (>1100 ng/mL; normal range <146 ng/mL) relative to other subjects in Cohort 3 (FIG. 4). Notably, maximal ARC1779 plasma concentrations in Subject 017 (8.3 μg/mL) were similar to maximal concentrations (8.3-10.6 μg/mL) measured in other individuals in Cohort 3. As shown in FIG. 5, no clinically significant elevations of C3a were observed in any of the successive cohorts (4-7) to whom ARC1779 was administered by the slow bolus method.

### Pharmacokinetics

An accurate, sensitive, reproducible and specific high performance liquid chromatography (HPLC) assay was developed for quantitative determination of ARC1779 concentrations in human plasma. The lower limit of quantification (LLOQ) of the HPLC assay is 0.25 μg/mL, with a concentration range from 0.25 to 200 μg/mL. Plasma concentrations of ARC1779 in Part A were determined from samples collected at Day 0 prior to drug administration, Day 0 post-drug administration (at ˜5, 15, 30, 60, 120, 180, 240, 480, 720 and 1440 minutes), Day 2 and Day 7 after IV push or slow bolus administration. All ARC1779 concentrations were reported based on oligonucleotide mass, excluding the mass of PEG.

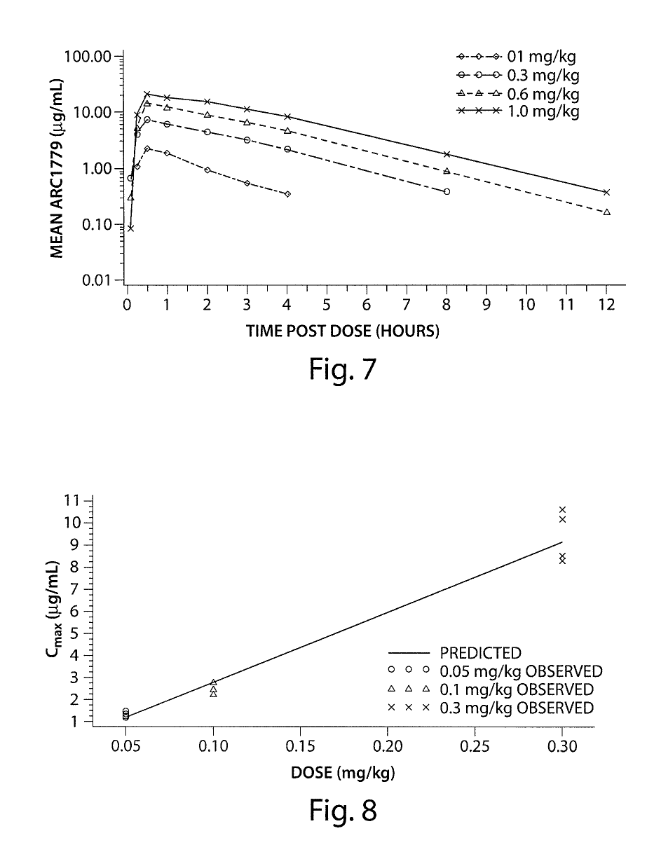

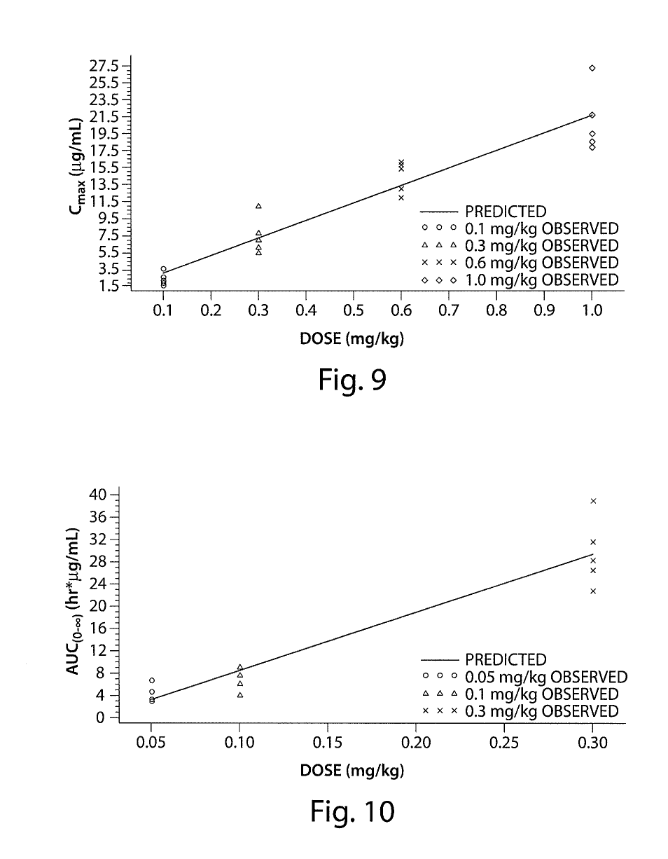

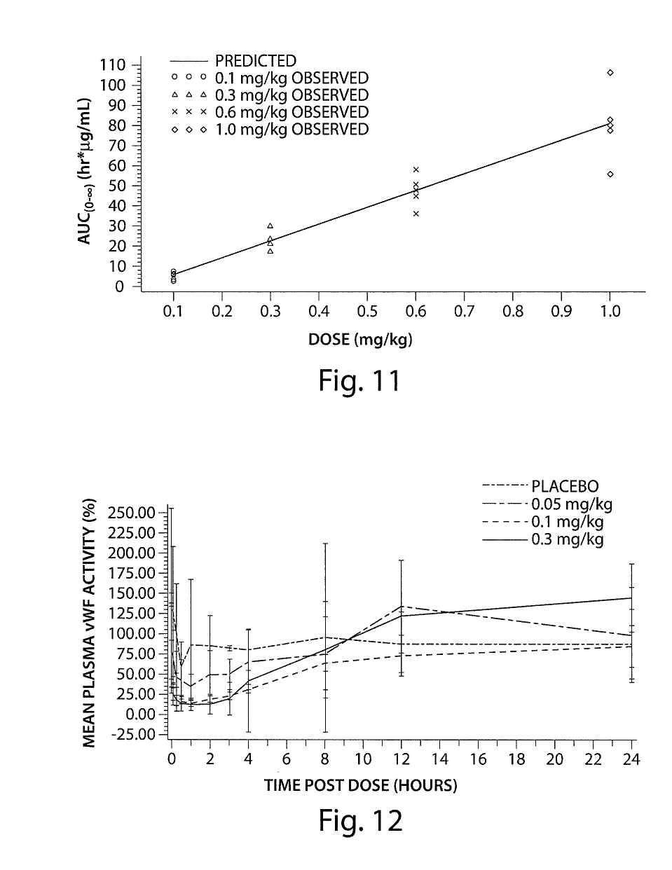

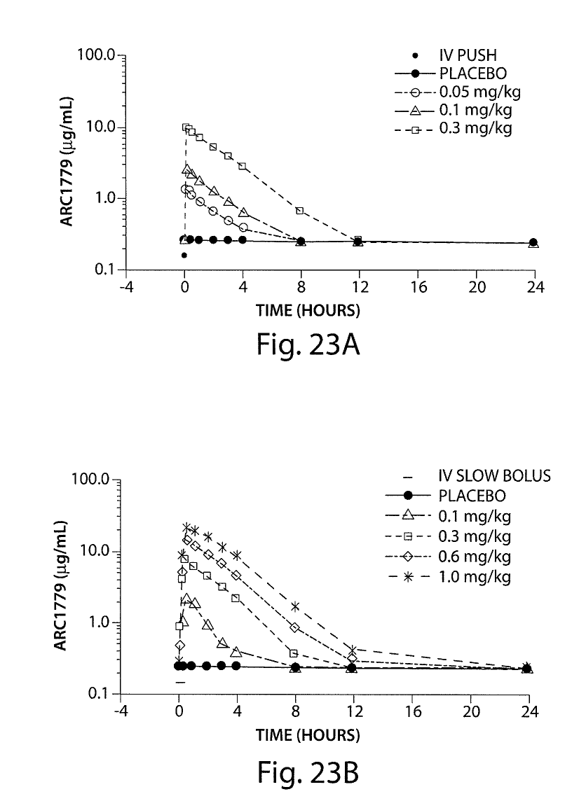

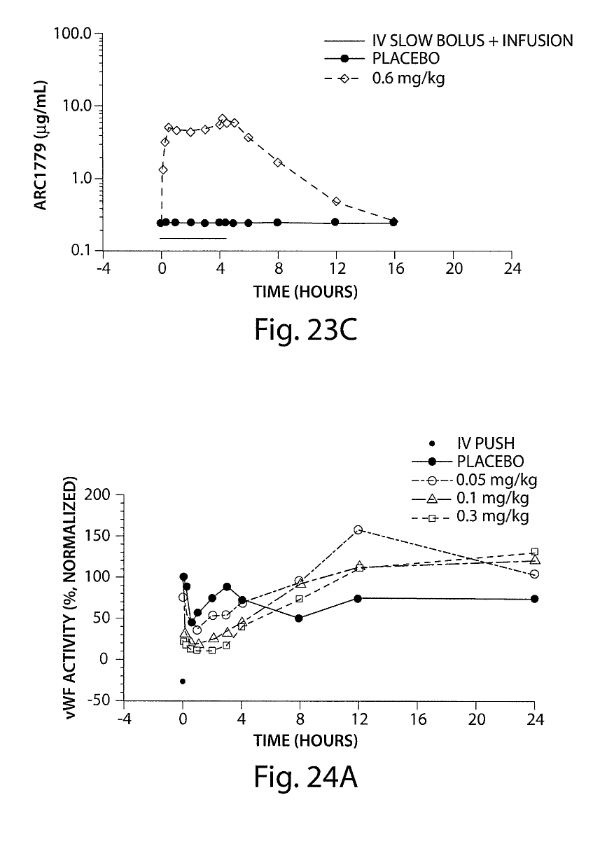

The mean plasma concentrations of ARC1779 over time for the 0.05, 0.1 and 0.3 mg/kg IV push dose groups, and 0.1, 0.3, 0.6 and 1.0 mg/kg slow bolus dose groups of Part A are shown in FIG. 6 and FIG. 7, respectively, and in FIG. 23. The concentration-time profiles of ARC1779 after IV push or slow bolus administration were monophasic. Non-compartmental analysis (NCA) of ARC1779 concentration-time profiles in Part A showed dose-proportional increases in mean Cₘₐₓ, AUC₍₀₋ₗₐₛₜ₎ and AUC_{(0-∞)}. The relationship between the dose of ARC1779 administered and increases in Cₘₐₓ or AUC_{(0-∞)} for the IV push and slow bolus groups are plotted in FIGS. 8, 9, 10 and 11.

Maximal exposure to ARC1779 was produced by the 1.0 mg/kg slow bolus administration, which gave a mean Cₘₐₓ of 21.15 μg/mL and a mean AUC_{(0-∞)} of 80.92 μg·hr/mL. Tₘₐₓ after IV push was 7-10 minutes, and 30 minutes after slow bolus administration. The apparent elimination half-life of ARC1779 (t_{1/2β)} was 2 hours, and mean residence time (MRT) was ˜3 hours. Mean volumes of distribution (V_{z} and Vₛₛ) across the dose range examined were ˜2-fold less than the blood volume in humans (˜74.29 mL/kg; Davies & Morris, 1993) indicating that ARC1779 was not widely distributed beyond the central compartment. Mean CL values after IV push or slow bolus administration ranged from ˜10% to ˜21% of the glomerular filtration rate (GFR) (˜107.14 mL/hr/kg; Davies & Morris, Pharm. Res., vol. 10(7): 1093-95 (1993)), suggesting that renal filtration is not the major route of clearance of ARC1779 in humans.

The complete set of PK parameter estimates for the 0.05, 0.1 and 0.3 dose groups (Cohorts 1-3) after IV push administration of ARC1779 are summarized in Table 2, and PK parameter estimates for the 0.1, 0.3, 0.6 and 1.0 mg/kg dose groups (Cohorts 4-7) after slow bolus administration of ARC1779 are summarized in Table 3.

TABLE 2
Mean PK Parameter Estimates for ARC1779 in Healthy Subjects after
Single-dose, IV Push Administration
Mean Pharmacokinetic Parameters for ARC1779 Following an IV Bolus
Cohorts 1-3 (by IV Push)
PK Analysis Set

| | | Doses Administered by IV Push | | | | | |
|---|---|---|---|---|---|---|---|
| Parameter | | 0.05 mg/kg | | 0.1 mg/kg | | 0.3 mg/kg | |
| Estimate | Unit | Mean | Std. Dev. | Mean | Std. Dev. | Mean | Std. Dev. |
| t ½, beta | hr | 2.4811 | 0.69748 | 2.0333 | 0.47990 | 2.1719 | 0.40298 |
| Tmax | hr | 0.1164 | 0.07468 | 0.1664 | 0.18649 | 0.1164 | 0.07468 |
| Cmax | μg/mL | 1.3724 | 0.10677 | 2.5464 | 0.23737 | 9.1746 | 1.13266 |
| NCmax | kg*μg/mL/μg | 0.0274 | 0.00212 | 0.0255 | 0.00236 | 0.0306 | 0.00378 |
| AUC(0-last) | hr*μg/mL | 3.1535 | 1.34376 | 5.2961 | 1.36226 | 27.0914 | 7.21074 |
| AUC(0-inf) | hr*μg/mL | 4.2582 | 1.58780 | 7.3107 | 2.03862 | 29.6811 | 6.09881 |
| NAUC(0-inf) | hr*kg*μg/mL/μg | 0.0852 | 0.03175 | 0.0731 | 0.02041 | 0.0989 | 0.02035 |
| AUC(Extrap) | % | 26.2944 | 7.05065 | 26.9007 | 5.38441 | 9.1101 | 10.66036 |
| Vz | mL/kg | 43.1155 | 5.44138 | 41.2589 | 7.29023 | 33.2446 | 12.01060 |
| Cl | mL/hr/kg | 12.8711 | 3.86699 | 14.7931 | 5.09679 | 10.4349 | 2.02387 |
| MRT(0-last) | hr | 1.7848 | 0.64034 | 1.4646 | 0.22154 | 2.3734 | 0.49623 |
| MRT(0-inf) | hr | 3.4987 | 0.95270 | 2.9119 | 0.69231 | 3.0375 | 0.26487 |
| Vss | mL/kg | 42.1606 | 4.48473 | 40.8242 | 6.58094 | 31.8761 | 8.08387 |

TABLE 3
Mean PK Parameter Estimates for ARC1779 in Healthy Subjects after
Single-dose, IV Slow Bolus Administration
Mean Pharmacokinetic Parameters for ARC1779 Following an IV Bolus
Cohorts 4-7 (by Slow Bolus)
PK Analysis Set

| | | Doses Administered by Slow Bolus | | | | | | | |
|---|---|---|---|---|---|---|---|---|---|
| Parameter | | 0.1 mg/kg | | 0.3 mg/kg | | 0.6 mg/kg | | 1.0 mg/kg | |
| Estimate | Unit | Mean | Std. Dev. | Mean | Std. Dev. | Mean | Std. Dev. | Mean | Std. Dev. |
| t ½, beta | hr | 1.5544 | 0.44082 | 1.6682 | 0.19650 | 1.7728 | 0.35237 | 1.8176 | 0.40262 |
| Tmax | hr | 0.6000 | 0.22361 | 0.4500 | 0.11180 | 0.5000 | 0.00000 | 0.5000 | 0.00000 |
| Cmax | μg/mL | 2.3344 | 0.76033 | 7.5156 | 2.14253 | 14.5660 | 1.87102 | 21.1542 | 3.77204 |
| NCmax | kg*μg/mL/μg | 0.0233 | 0.00761 | 0.0250 | 0.00713 | 0.0243 | 0.00311 | 0.0211 | 0.00377 |
| AUC(0-last) | hr*μg/mL | 4.0980 | 1.53140 | 22.1543 | 4.70285 | 46.4213 | 7.88129 | 79.3905 | 18.29128 |
| AUC(0-inf) | hr*μg/mL | 5.1019 | 1.97499 | 23.1015 | 4.61833 | 47.7908 | 8.10046 | 80.9190 | 17.92570 |
| NAUC(0-inf) | hr*kg*μg/mL/μg | 0.0510 | 0.01979 | 0.0770 | 0.01538 | 0.0797 | 0.01350 | 0.0809 | 0.01794 |
| AUC(Extrap) | % | 18.9986 | 7.82097 | 4.2885 | 1.37134 | 2.8658 | 0.58022 | 2.0365 | 1.35762 |
| Vz | mL/kg | 48.0464 | 16.19813 | 32.7564 | 9.97520 | 32.2486 | 4.42714 | 33.4923 | 8.57888 |
| Cl | mL/hr/kg | 22.6209 | 10.04313 | 13.3952 | 2.61240 | 12.8683 | 2.34346 | 12.8854 | 3.06112 |
| MRT(0-last) | hr | 1.3129 | 0.19861 | 2.3887 | 0.03766 | 2.6280 | 0.39114 | 2.8850 | 0.30431 |
| MRT(0-inf) | hr | 2.2145 | 0.65232 | 2.7353 | 0.15189 | 2.8934 | 0.41962 | 3.1022 | 0.37735 |
| Vss | mL/kg | 47.3362 | 15.77622 | 36.9275 | 9.06515 | 36.6806 | 4.49484 | 39.4621 | 7.18640 |

In summary, the concentration-time profiles for ARC1779 after IV push or slow bolus appeared monophasic, though the terminal phase may not have been fully captured. The Cₘₐₓ and AUC values were dose-proportional. The highest exposure was observed after 1.0 mg/kg slow bolus, with mean Cₘₐₓ of 21.15 μg/mL and AUC_{(0-∞)} of 80.92 μg-hr/mL. The mean apparent elimination half-life (t_{1/2β)} was ˜2 hours, and mean residence time (MRT) was 3 hours. The mean apparent volumes of distribution (V_{z} and Vₛₛ) were ˜½ of the blood volume, suggesting that ARC1779 distribution is in the central compartment. The mean clearance (CL) values ranged from ˜10% to 21% of the glomerular filtration rate (GFR), suggesting that renal filtration may not be a major mechanism of clearance of ARC1779.

### Pharmacodynamics

The pharmacodynamic parameters assessed in Part A of the study included evaluation of the effects of ARC1779 on vWF activity and platelet function (i.e., vWF-mediated, high shear force-dependent platelet aggregation). Measurement of vWF activity was performed by quantifying vWF A1-domain binding activity in plasma using an enzyme-linked immunosorbent assay (ELISA). Measurement of platelet function in whole blood was assessed using the platelet function analyzer (PFA-100®) assay. Data from the ELISA and PFA-100® assays for the 0.05, 0.1 and 0.3 mg/kg IV push dose groups (Cohorts 1-3), and 0.05, 0.1, 0.3, 0.6 and 1.0 mg/kg slow bolus dose groups (Cohorts 4-7) of Part A are presented below. The PK/PD relationship of ARC1779 concentration to inhibition of vWF activity and platelet aggregation is also discussed below.

### Inhibition of vWF Activity

The inhibitory effect of ARC1779 on plasma vWF activity [a measure of the amount of active (“free”) vWF with functional A1 domain present in plasma] was evaluated with a commercially available, quantitative, direct ELISA kit. Plasma vWF antigen concentration, a measure of the total amount of vWF present in plasma, was also evaluated with a commercially available, quantitative, sandwich ELISA kit with results reported as mU/mL (data not shown).

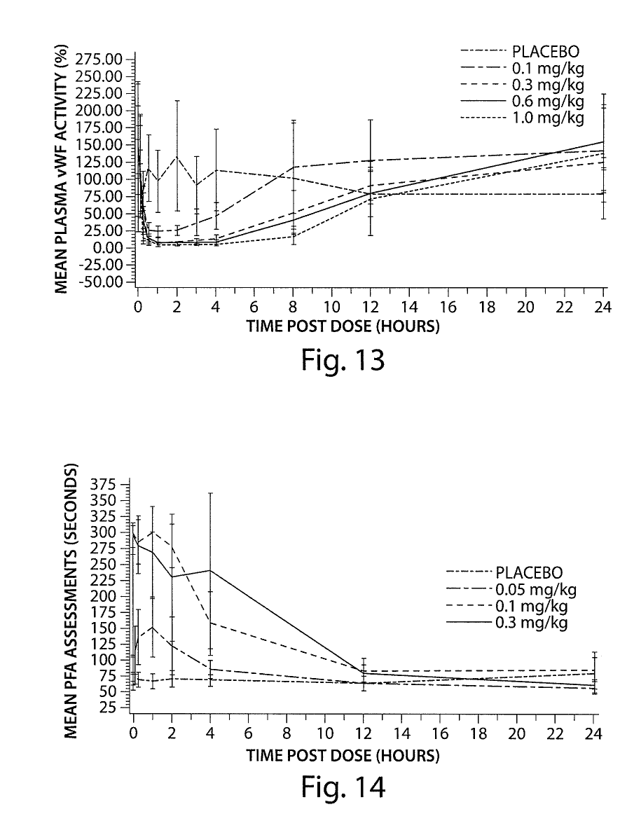

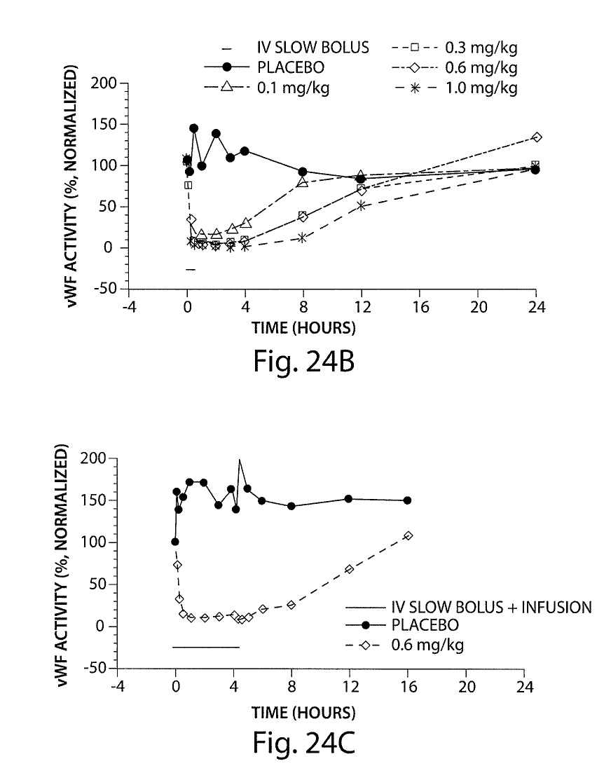

vWF activity was evaluated from samples collected at Day 0 prior to drug administration, Day 0 post-drug administration (at ˜5, 15, 30, 60, 120, 180, 240, 480, 720 and 1440 minutes), Day 2 and Day 7. Plots of the mean percentage (%) of vWF activity over time for the 0.05, 0.1 and 0.3 mg/kg IV push dose groups (Cohorts 1-3), and 0.1, 0.3, 0.6 and 1.0 mg/kg slow bolus dose groups (Cohorts 4-7) of Part A are shown in FIGS. 12 and 13, respectively, and in FIG. 24.

vWF activity was inhibited in a dose-dependent manner after single-dose IV push or slow bolus administration with rapid onset of action and gradual restoration of vWF activity by 12-24 hours post-dose. Near complete inhibition of vWF activity (to ≦3% of baseline or the LLOQ of the assay) was achieved and sustained for at least 4 hours with 1.0 mg/kg of ARC1779. Virtually complete inhibition (to ˜5% of baseline) was achieved and maintained for nearly this same duration with 0.6 mg/kg of ARC1779.

### Inhibition of Platelet Aggregation (PFA-100®)

The effect of ARC1779 on vWF-mediated platelet function was evaluated using the PFA-100® instrument. The PFA-100® assay simulates platelet-related primary hemostasis in vivo by evaluating platelet adhesion and aggregation in whole blood in vitro. The value reported is termed the closure time and represents the elapsed time in seconds until closure (i.e., formation of a hemostatic plug in the microscopic aperture of the instrument) with maximal closure time of 300 seconds. PFA-100® closure time was measured in samples collected at Day 0 prior to drug administration and Day 0 post-drug administration (at ˜5, 15, 60, 120, 240, 480, 720, and 1440 minutes).

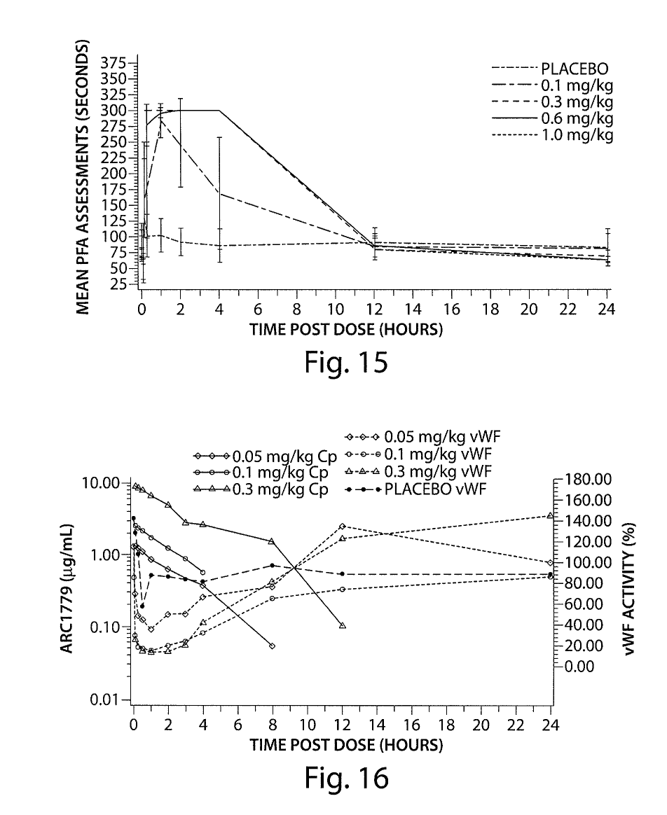

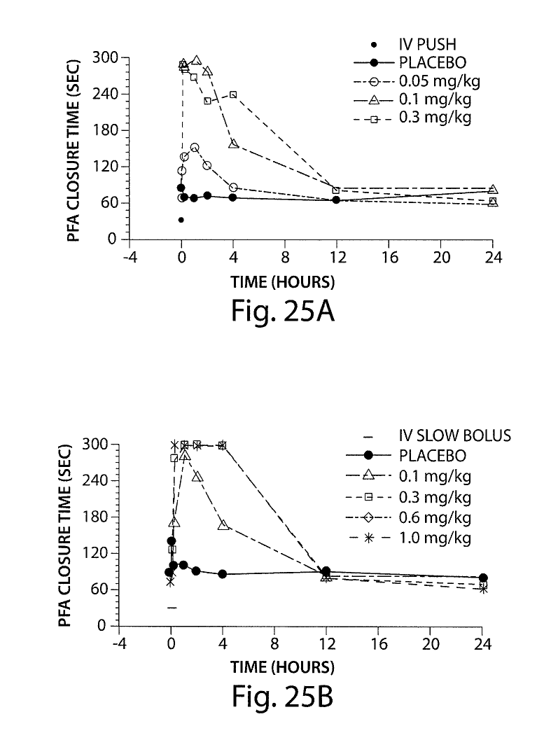

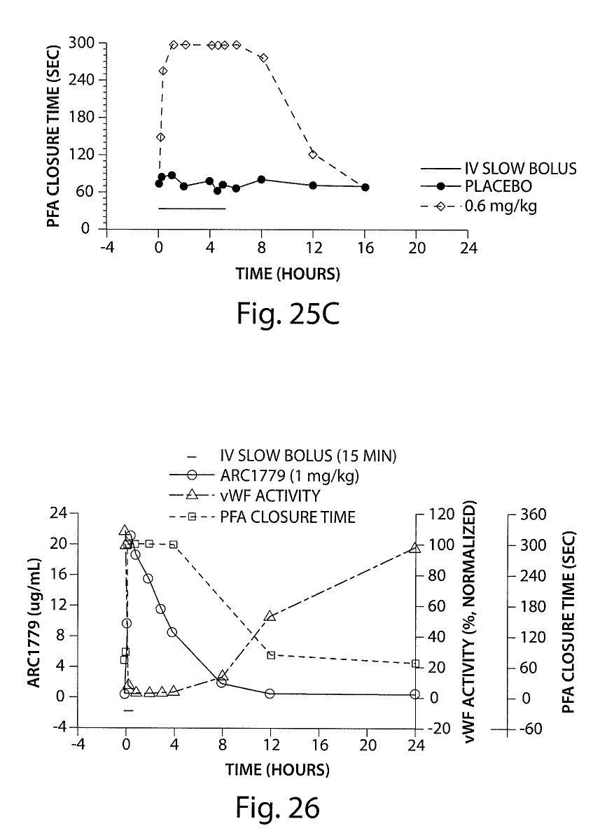

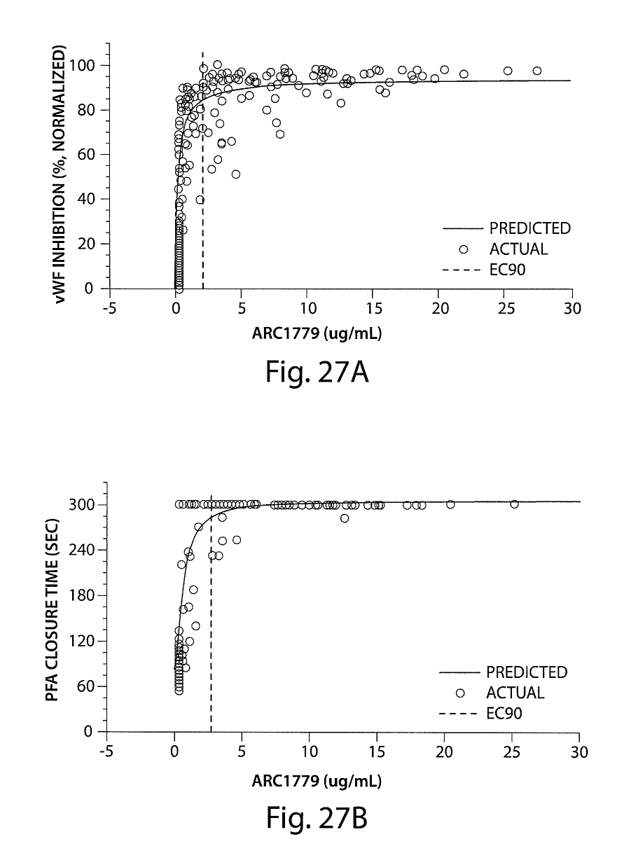

Plots of mean PFA-100® closure times for the 0.05, 0.1 and 0.3 mg/kg IV push dose groups (Cohorts 1-3), and the 0.1, 0.3, 0.6 and 1.0 mg/kg IV slow bolus dose groups (Cohorts 4-7) of Part A are shown as a function of time in FIGS. 14 and 15, respectively, and in FIG. 25. Platelet function as assessed by prolongation of PFA-100® closure time was inhibited in a dose-dependent manner up to a dose level of 0.3 mg/kg. At doses higher than 0.3 mg/kg, prolongation of PFA-100® closure times became saturated with complete inhibition of platelet function for ˜4 hours. Restoration of platelet function occurred by 8-12 hours post-dose.

### Pharmacokinetic and Pharmacodynamic Relationship

The PK/PD relationship between ARC1779 plasma concentration and the extent of inhibition of vWF activity was evaluated for the IV push (Cohorts 1-3) and IV slow bolus dose groups (Cohorts 4-7). vWF activity was inhibited in a dose-dependent and concentration-dependent manner following single-dose administration of ARC1779 via the IV push and slow bolus methods.

After a single IV push at 0.05, 0.1 or 0.3 mg/kg (Cohorts 1-3), the extent of inhibition of vWF activity ranged from ˜24 to 60%, 62 to 83% and 63 to 89% at Tₘₐₓ (5 minutes post-dose) to 4 hours post-dose with mean Cₘₐₓ of 1.37, 2.55 and 9.17 μg/mL, respectively. vWF activity returned to baseline in a dose-dependent manner, with full or near-full activity restored (>70%) by 8 hours post-dose for all dose groups (FIGS. 16 & 26).

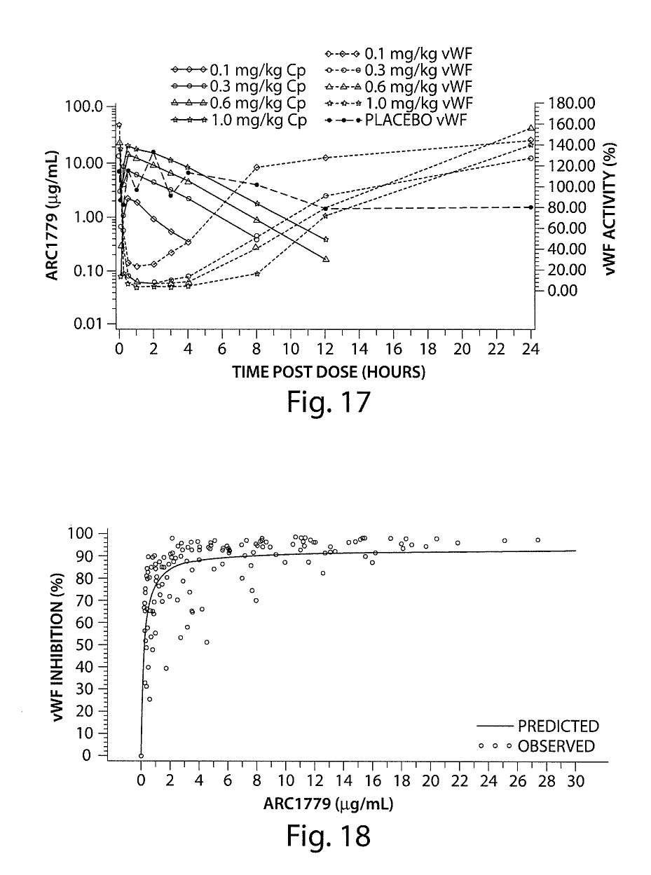

After a single slow bolus at 0.1, 0.3, 0.6 or 1.0 mg/kg (Cohorts 4-7), the extent of inhibition of vWF activity ranged from ˜67 to 83%, 89 to 90%, 94 to 96% and 96 to 98% at Tₘₐₓ (0.5 hours post-dose) to 4 hours post-dose with mean Cₘₐₓ from 0.11 to 2.21 μg/mL, 2.19 to 7.42 μg/mL, 4.68 to 14.57 μg/mL and 8.49 to 21.15 μg/mL, respectively. vWF activity returned to baseline in a dose-dependent manner. By 12 hours post-dose, 100%, 77%, 56% and 46% of vWF activity was restored for the 0.1, 0.3, 0.6 and 1.0 mg/kg, respectively, and by 24 hours post-dose full activity was restored for all groups (FIGS. 17 & 26).

The relationship of ARC1779 concentration to inhibition of vWF activity was analyzed by Eₘₐₓ modeling, as shown in FIGS. 18 & 27.

The fitted EC₅₀ and EC₉₀ values for inhibition of vWF activity after single-dose administration of ARC1179 were 0.22 μg/mL (17 nM) and 1.98 μg/mL (151 nM), respectively.

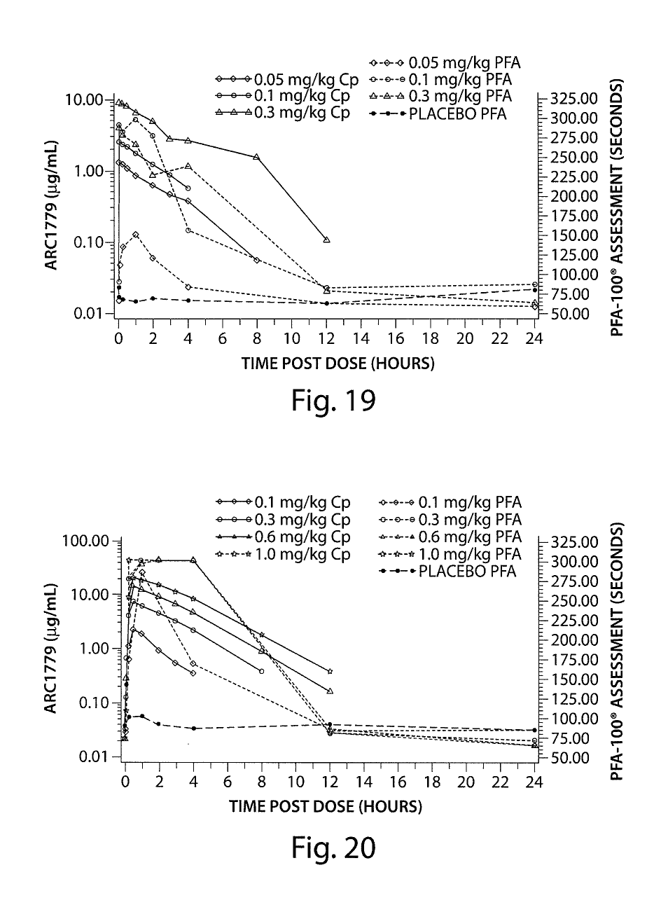

The PK/PD relationship between ARC1779 plasma concentration and inhibition of platelet function was similarly evaluated by measurement of PFA-100® closure times for the IV push (Cohorts 1-3) and IV slow bolus dose groups (Cohorts 4-7). PFA-100® closure times were prolonged in a dose-dependent manner following IV push administration of ARC1779 at 0.05, 0.1 and 0.3 mg/kg (Cohorts 1-3). Closure time showed a modest increase relative to baseline at mean Cₘₐₓ (5 minutes post-dose) of 1.37 μg/mL after a single, IV push of ARC1779 at 0.05 mg/kg. At 0.1 or 0.3 mg/kg kg, Cₘₐₓ values (5 minutes post-dose) were 2.55 and 9.17 μg/mL, respectively, and closure times became saturated with complete inhibition of platelet function for ˜2 or ˜4 hours and returned to baseline by ˜12 hours post-dose (FIG. 19).

After slow bolus administration of ARC1779 at 0.1 mg/kg, ARC1779 PFA-100® closure times saturated at mean Cₘₐₓ(1 hour post-dose) of 1.88 μg/mL and platelet function was completely inhibited for ˜1 hour. Slow bolus administration of ARC1779 at 0.3, 0.6 and 1.0 mg/kg was associated with saturation of closure times by 15 minutes post-dose, and complete inhibition of platelet function for ˜4 hours, with return to baseline by 12 hours post-dose (FIG. 20).

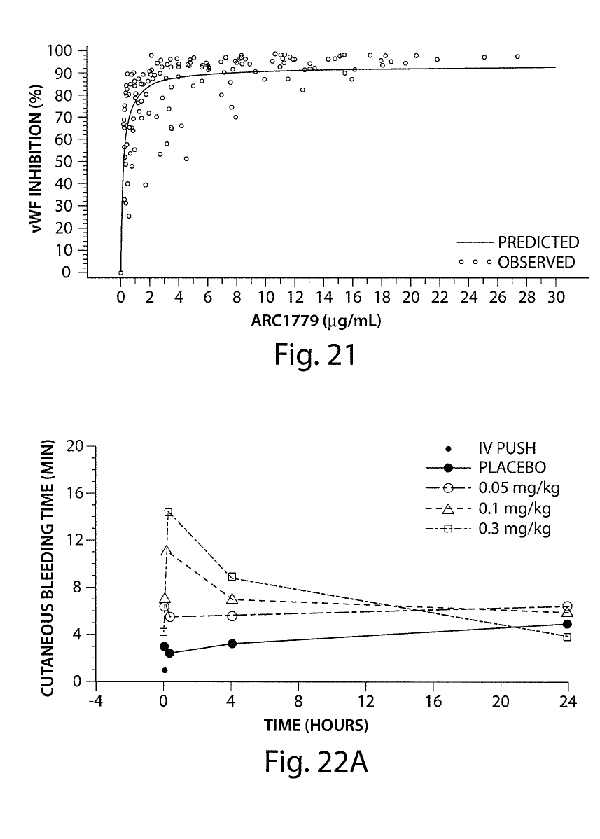

By Eₘₐₓ modeling, the fitted EC₅₀ and EC₉₀ values for platelet function inhibition (as assessed by PFA-100® closure time) after single-dose administration of ARC1779 were 0.75 μg/mL (57 nM) and 2.57 μg/mL (196 nM), respectively (FIGS. 21 & 27).

In summary, inhibition of vWF A1 binding was achieved in a dose-dependent and concentration-dependent manner, with respective EC₅₀ and EC₉₀ values of 0.22 μg/mL (17 nM) and 1.98 μg/mL (151 nM). Platelet function inhibition (PFA-100® closure time) was achieved, with respective EC₅₀ and EC₉₀ values of 0.75 μg/mL (57 nM) and 2.57 μg/mL (196 nM). vWF activity returned in a dose-dependent and concentration-dependent manner.

### Example 2

### Functional Cell Assays

The effectiveness of various aptamers in blocking vWF function in several biological assays is described in this Example.

In one assay, botrocetin is used. Botrocetin, a protein isolated from snake venom, is known to induce von Willebrand Factor binding to the gpIb receptor on live and fixed platelets. This reaction causes agglutination of suspensions of fixed platelets via vWF multimerization. In preparations of platelet rich plasma (hereinafter “PRP”), vWF/botrocetin induction of agglutination is followed by a second phase of platelet aggregation caused by metabolic activation of the platelets. These two reactions: vWF binding to fixed platelets and vWF mediated platelet aggregation, can be used to measure the activity of aptamers of the invention.

The amount of vWF bound to fixed platelets can be measured with an antibody to vWF. The fluorescence signal from bound antibody incubated with a fluorescein conjugated secondary antibody is then detected and quantified by flow cytometry. The ability of an aptamer to block vWF binding to platelets is correlated with a reduction in fluorescence signal.

Botrocetin induces the binding of the A1 domain as well as the full length protein of vWF to platelets. It was determined by the inventors that 6-Histidine-tagged rabbit A1 domain vWF purified protein could be induced to bind to human lyophilized platelets with botrocetin. Rabbit A1 binding to platelets is measured with an anti-poly-His antibody followed by incubation with a phycoerythrin conjugated secondary antibody. The degree of binding can be quantified by flow cytometric analysis. The ability of aptamers to block the binding of rabbit A1 to human fixed platelets was correlated with decreased fluorescence signal.

In platelet rich plasma isolated from fresh human blood, botrocetin induces platelet aggregation via vWF. Platelet aggregate formation can be measured optically as an increase in percent light transmittance on a Chronolog Model 490-4D Aggregometer because aggregation of platelets clarifies the plasma. Aptamers were analyzed for their ability to inhibit botrocetin induced platelet aggregation (“BIPA”) in human blood. An aptamer was considered to be active if it could prevent aggregate formation for six minutes post botrocetin addition.

Another assay is an agonist independent but vWF dependent assay that uses the PFA-100® instrument (Harrison et al., Clin. Lab. Haem., v24:225-32 (2002)). The PFA-100® simulates the formation of a hemostatic plug under conditions of high shear force in vivo by recording the time required for platelets to aggregate and block the flow of citrated whole blood through a microscopic aperture in a membrane coated with collagen and either epinephrine or ADP. This activity is von Willebrand factor dependent as high MW vWF multimers bind to immobilized collagen on the membrane and then bind to and activate platelets because of the shear force induced by drawing the blood through the microscopic aperture. Thus, this assay is complimentary to the BIPA and FACS assays in that it is vWF dependent, however it has some advantages in that it does not require the addition of the vWF agonist botrocetin and uses whole blood instead of platelet rich plasma.

Another assay uses ADP to induce platelet aggregation. Aggregation of platelet rich plasma (PRP) can be in induced in multiple ways. The snake venom protein botrocetin acts on vWF as described above, stabilizing its interaction with the platelet receptor gplb, thereby inducing platelet aggregation. Binding of vWF to gplb is an early step in platelet aggregation, thus there is an expectation that inhibitors that block downstream components of the aggregation process (i.e.; the IIbIIIa antagonists INTEGRILIN® and REOPRO® would also prevent botrocetin induced platelet aggregation). However, in the case of agonists that act directly on platelets and induce aggregation (ADP for example), one would expect that antagonists upstream of the agonist would be ineffective (an anti-vWF aptamer for example), while antagonists that act directly on platelets (IIbIIIa antagonists) would remain potent. The specificity of a vWF antagonist relative to a IIbIIIa antagonist will increase the safety of the anti-vWF antagonist by decreasing the bleeding time associated with treatment. For patients with atherosclerotic plaques in stenosed arteries, platelet aggregation occurs as platelets bind to collagen immobilized vWF on the surface of the plaque. Thus, both inhibiting the vWF/gpIb interaction as well as blocking the IIbIIIa receptor binding to fibrin will prevent platelet aggregation. The biological specificity conferred by targeting vWF insures that, unlike anti-IIbIIIa treatment, platelets themselves are not targeted directly, insuring they can still be activated by other means, thus reducing potential bleeding complications associated with anti-platelet therapy.

### Example 3

### Aptamer-Mediated Inhibition of von Willebrand Factor-Mediated Platelet Function with Platelets from Patients with Acute Myocardial Infarction

ARC1779, an aptamer that blocks the binding of the A1 domain of von Willebrand Factor (vWF) to the platelet GPIb receptor, was used to treat platelets collected from acute myocardial infarction (MI) patients. vWF is increased in the elderly and in the setting of MI, as reflected in higher vWF levels in circulation and in increased shear-dependent platelet function, as measured by the platelet function analyzer (PFA-100®) and cone and plate analyzer (IMPACT@). Conventional pharmacotherapy of myocardial infarction partially reduces platelet activation and aggregation, but does not address excessive vWF activity or platelet adhesion.

In the studies described herein, the dose response curves for ARC1779 on platelet function tests (PFA-100® and IMPACT®), agonist-induced platelet aggregation and vWF activity (free A1 domain sites) of patients with acute myocardial infarction (AMI) on standard treatment including aspirin and clopidogrel (n=40), young (n=20) and elderly controls (n=20) were evaluated.

ARC1779 fully blocked collagen adenosine diphosphate (ADP) induced platelet plug formation ex vivo, as measured by PFA-100®, with an IC₁₀₀ of approximately 1-2 mcg/mL with citrate anticoagulation and 3-5 mcg/mL with hirudin anticoagulation.

ARC1779 fully blocked shear-dependent platelet adhesion, as measured by the IMPACT® analyzer, with an IC₁₀₀ of approximately 1 mcg/mL with citrate anticoagulation. In contrast to GPIIb/IIIa antagonists, ARC1779 did not inhibit platelet aggregation by ADP, collagen or arachidonic acid at concentrations (10 mcg/mL) that fully inhibited vWF dependent platelet function. ARC1779 fully blocked ex vivo vWF activity with an IC₉₀ of approximately 1 mcg/mL in young controls and 6-8 mcg/mL in STEMI and NSTEMI patients.

The results of these studies are summarized in the Table 4 below.

TABLE 4

| ARC1779 (mcg/mL) | Young Controls | Elderly Controls | NSTEMI | STEMI |
|---|---|---|---|---|
| IC₁₀₀ PFA: Citrate | 0.9 +/− 0.5 | 1.7 +/− 0.9 | 0.9 +/− 0.9 | 1.2 +/− 0.4 |
| Mean +/− SD | | | | |
| IC₁₀₀ PFA: Hirudin | 3.0 +/− 1.6 | 4.7 +/− 2.0 | 2.9 +/− 2.4 | 4.5 +/− 1.1 |
| Mean +/− SD | | | | |
| IC₁₀₀ IMPACT | 0.6 +/− 0.4 | 0.7 +/− 0.1 | 1.4 +/− 0.4 | 0.9 +/− 0.5 |
| Mean +/− SD | | | | |
| IC₉₀ vWF activity | 1.8 +/− 0.8 | NA | 7.5 +/− 8.8 | 6.4 +/− 5.2 |
| Mean +/− SD | | | | |

ARC1779 is a potent and specific inhibitor of vWF activity and vWF dependent platelet function, even in the setting of AMI where vWF activity is increased. Thus, vWF is a target for AMI therapy and ARC1779 is useful as a vWF antagonist in the treatment of patients with acute myocardial infarction.

### Example 4

### Treatment Regimen for Use of Anti-vWF Antagonist Aptamers in Treatment of Patients with Acute Coronary Syndrome

ARC1779, a potent and specific antagonist of von Willebrand Factor (vWF), is used in patients diagnosed with acute coronary syndromes (ACS) who are undergoing percutaneous coronary intervention (PCI) procedures. vWF function impacts thrombotic diseases, such as ACS, by recruiting and activating platelets to vascular lesions and forming a bridge between exposed collagen on the wall of a damaged vessel and GPIb on platelets via its A1 domain.

Formulations of ARC1779 are administered in patients with ACS undergoing PCI and the ability of these formulations to improve myocardial perfusion while also providing therapeutically effective anti-thrombotic treatment at the primary site of arterial blockage is evaluated. For example, a patient's myocardial perfusion is improved by reducing blood clots in the microcirculation.

Evaluation of ARC1779 injection as an anti-platelet agent for use during percutaneous coronary intervention (PCI) procedures in patients with acute coronary syndromes (ACS) was performed in a Phase 2a clinical trial. That trial, a multi-center, randomized, double-blind, dose-escalation study, was terminated prematurely as a result of the Sponsor's decision to discontinue development of ARC1779 injection for the ACS indication. ARC1779 injection or comparator drug (REOPRO®) was administered to 18 ACS patients with acute non-ST-elevation myocardial infarction (NSTEMI). Patients were treated with ARC1779 injection at the lowest planned dose (0.1 mg/kg) as an IV slow bolus given over 15 minutes. Serious adverse events (SAEs) that were unrelated to study drug administration were reported in one patient who died as a result of worsening chronic obstructive pulmonary disease. No hypersensitivity reactions occurred among ACS patients treated with ARC1779 injection. Preliminary data from a subset of patients suggest that the 0.1 mg/kg dose was associated with a mean ARC1779 plasma concentration of ˜1.5 mcg/mL and ˜70% inhibition of VWF activity measured immediately post-PCI.

### Example 5

### Aptamer-Mediated Inhibition of von Willebrand Factor-Mediated Ex Vivo Platelet Function in Thrombotic Thrombocytopenic Purpura (TTP)

ARC1779 is an aptamer that blocks the binding of the vWF A1 domain to platelet GPIb receptors. In thrombotic thrombocytopenic purpura (TTP), there is an excess of ultra-large multimers of vWF, which are especially avid for binding GPIb and give rise to disseminated platelet thrombi that are fibrin-poor and vWF-rich in composition. ARC1779 is used as front-line therapy of acute TTP in conjunction with plasma exchange. ARC1779 has already been shown in healthy volunteers to inhibit vWF activity and vWF-dependent platelet function. ARC1779 has no anticoagulant effect and does not inhibit other pathways of platelet activation. Without intending to be bound by theory, ARC1779 is expected to normalize platelet dysfunction and prevent the thrombotic end-organ complications of TTP based upon the mechanism of action defined for ARC1779 and the mechanism of thrombosis defined for TTP.

vWF activity (vWF:RiCO) and platelet function were assessed in blood samples taken from TTP patients and age-matched, healthy controls. The ex vivo dose response curves for ARC1779 on vWF activity (free A1 domain sites) and on platelet function assessed by the platelet function analyzer (PFA-100®), cone and plate analyzer (IMPACT®) and agonist-induced impedance platelet aggregometry (MULTIPLATE®) of TTP patients (N=10, 3 in acute phase and 7 in remission) and healthy age-matched controls (N=23) were studied.

vWF:RiCO activity (p=0.002) and vWF-dependent platelet plug formation (p=0.001) were increased in TTP patients relative to healthy controls, but agonist-induced platelet aggregation (ADP, arachidonic acid, TRAP) was not. ARC1779 fully blocked platelet plug formation, as measured by PFA-100®, with an IC₁₀₀ of ˜1 mcg/mL with citrate anticoagulation and ˜3-4 mcg/mL with hirudin anticoagulation in both TTP patients and in healthy controls. ARC1779 fully blocked shear-dependent platelet adhesion, as measured by the IMPACTS analyzer, with an IC₁₀₀ of ˜1 mcg/mL with citrate anticoagulation in both TTP patients and in healthy controls. ARC1779 fully blocked vWF activity (free A1 domain sites) with an IC₉₀ of ˜6 mcg/mL in TTP patients and ˜2 mcg/mL in young controls (p<0.001 between groups). ARC1779 did not inhibit platelet aggregation by ADP or arachidonic acid at concentrations (10 mcg/mL) that fully inhibited vWF dependent platelet function.

The results of these studies are summarized in the Table 5 below.

TABLE 5

| | Healthy Controls | TTP Patients |
|---|---|---|
| | (N = 23) | (N = 10) |
| | Mean +/− SD | Mean +/− SD |
| vWF: RiCO (%) | 94.9 +/− 60.4 | 153.3 +/− 55.9 |
| PFA-100 ® Closure Time in Citrate (sec) | 90.9 +/− 16.0 | 66.8 +/− 12.7 |
| PFA-100 ® Closure Time in Hirudin (sec) | 84.0 +/− 12.9 | 64.6 +/− 11.9 |
| ARC1779 IC₁₀₀ PFA in Citrate (ARC 1779 mcg/mL) | 0.9 +/− 0.4 | 1.4 +/− 0.6 |
| ARC1779 IC₁₀₀ PFA in Hirudin (ARC 1779 mcg/mL) | 3.2 +/− 1.5 | 4.4 +/− 2.7 |
| IC₁₀₀ IMPACT in Citrate (ARC1779 mcg/mL) | 0.8 +/− 1.2 | 0.8 +/− 0.8 |
| IC₉₀ vWF free A1 domain sites (ARC 1779 mcg/mL) | 1.8 +/− 0.8 | 6.2 +/− 2.7 |

ARC1779 potently and specifically inhibits vWF activity and vWF dependent platelet function in the setting of TTP where vWF activity is increased. Thus, vWF is a target for TTP therapy and ARC1779 is useful as a vWF antagonist in the treatment of TTP.

### Example 6

### Use of ARC1779 in Conjunction with Plasma Exchange in Patient with Acute Thrombotic Thrombocytopenic Purpura (TTP)

In this study, a patient presenting with acute TTP was treated with ARC1779 on a named patient basis (compassionate use) in accordance with standard procedures in Europe where medical practitioners can request and use certain drug product candidates prior to their approval by the applicable regulatory authorities when there is an unmet clinical need and the practitioners are satisfied that the use of the product candidate would provide a direct benefit to the patient.

ARC1779 was administered in conjunction with daily plasma exchange to a single patient with TTP beginning on Day 1. The final course of ARC1779 was administered on Day 24. During this course of treatment, a sustained rise in the patient's platelet count and a reduction in the levels of biomarkers (LDH, bilirubin and troponin) associated with cellular damage in the circulatory system was observed. In addition, vWF activity decreased during treatment. These data demonstrate that ARC1779 interfered with the disease process, reducing the excessive vWF activity and resulting platelet aggregation that is the hallmark of acute TTP.

### BACKGROUND

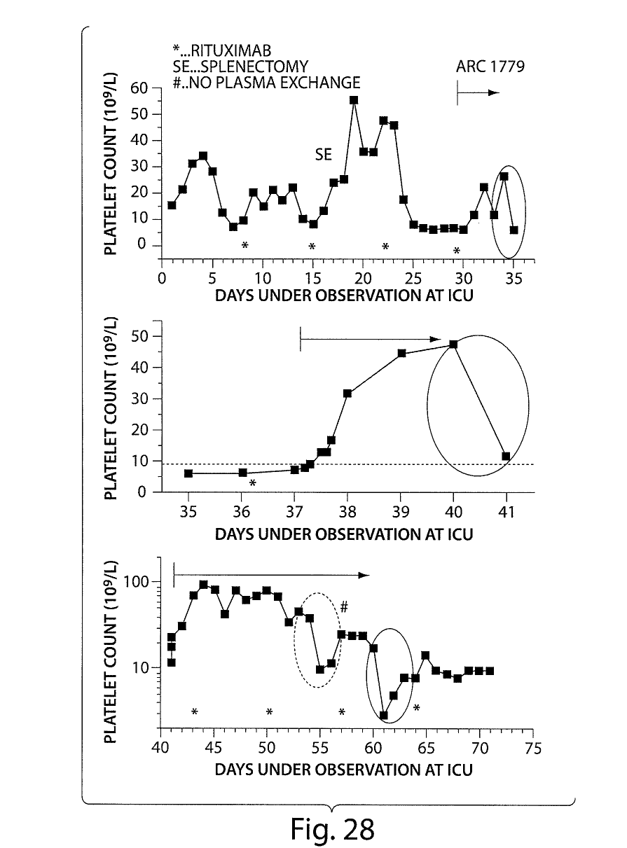

A 39 year old male patient (95 kg) was first admitted to another hospital because of symptoms of angina pectoris on Sep. 3, 2007. The initial lab values showed severe thrombocytopenia (13/nL), haemolytic anemia (hemoglobin 9.9 g/dL) with red cell fragmentation, and elevated lactate-dehydrogenase (LDH: 1171 U/dL), creatinine (2.5 mg/dL) and troponin T. It was 26 days before acute thrombotic thrombocytopenic purpura (TTP) was suspected. The patient was transferred to the University Hospital of Vienna and daily plasma exchange with 4000 mL OCTAPLAS®, (Octapharma, Vienna, Austria) was started on September 29^{th} (considered day 1 in the following chronology of events). At this time the patient was unconscious and had severe organ dysfunction (creatinine 1.23 mg/dL, troponin T 0.15 ng/mL, several episodes of bradycardia (HR<40/min), T-wave inversion on the ECG, elevated neurone specific enolase). He was intubated to prevent aspiration. ADAMTS13 activity and antigen levels (Technozym ADAMTS-13, Technoclone, Vienna, Austria) were below the detection limit (<0.02 U/mL; normal 0.5-1.1 U/mL), and a high titer anti-ADAMTS13 antibody (Technozym ADAMTS-13 INH) was found (233 U/mL; normal <12 U/mL). Under daily plasma exchange therapy and methylprednisone (100 mg/day) only a transient increase in platelet counts (max. 60/nL) were observed, but LDH remained elevated (FIGS. 28 & 29). Despite treatment escalation (twice daily plasma exchange, rituximab (4 times 375 mg/sqm/week started on day 8) and splenectomy on day 17, platelet counts remained low (FIGS. 28 & 29) and organ dysfunction persisted.

### Intervention

Currently there are no drugs authorized specifically for the treatment of acute TTP, but the ability of rituximab to prevent relapsing TTP is currently being tested in clinical trials. The investigational anti von Willebrand Factor (vWF) aptamer ARC1779 effectively inhibits vWF activity in plasma samples of TTP patients. As this would offer a new treatment possibility for our critically ill patient, we started therapy with ARC1779 according to §8 of the Austrian Drug Law for the emergency treatment of a named patient. This therapy was approved by the medical director of the hospital and the patient's relatives.

### Results & Discussion

### Clinical Course

While continuing daily plasma exchange treatments, the patient also received a bolus-primed, continuous intravenous infusion of ARC1779 at a rate of 2 μg/kg/min beginning on day 30, which was increased to 3.5 μg/kg/min after 1 day. The platelet counts increased slightly from 7 to a maximum of 30/nL after 91 hours of treatment with ARC1779 (FIG. 28), during which time Enlerococci septicaemia with elevated acute phase reactants was present.

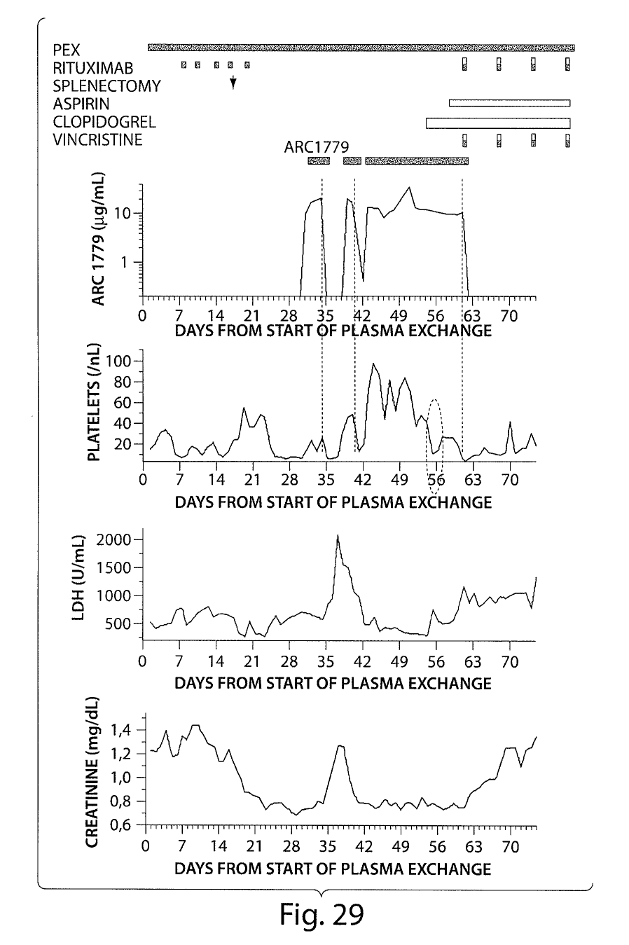

The infusion was stopped for safety reasons after 96 h (day 34), because bloody fluid was noted in the nasogastric tube. Gastroscopy revealed only slight mucosal inflammation and no further evidence of bleeding. After suspension of the ARC1779 infusion, platelet counts dropped to 5/nL in parallel with declining ARC1779 plasma concentrations (FIG. 28) and resulting loss of vWF-inhibition 16 h after suspension of infusion. In parallel, creatinine levels increased from 0.7 to 1.4 mg/dL, and LDH from 460 to 2254 U/L (FIG. 29), indicating exacerbation of thrombotic microangiopathy.

The continuous infusion of ARC1779 (2 μg/kg/min) was re-started on day 37 (FIGS. 28 & 29) without a recurrence of overt bleeding. Platelet counts increased from 9 to 45/nL by 38 h after resuming the infusion (FIG. 28) and creatinine levels returned to normal (FIG. 29). Due to a temporary lack of drug supply, the dose of ARC1779 was tapered to 1 μg/kg/min and stopped after 78 h. During this period, the platelet counts decreased to 12/nL by 12 h after stop of the infusion (FIG. 28). When ARC1779 was re-started, platelet counts increased to a maximum of 97/nL (FIG. 28) and LDH dropped to 264 U/mL (FIG. 29). The patient's neurologic state and level of consciousness continuously improved.

Additional therapy consisted of 4 further cycles of rituximab together with vincristine (2 mg/week) starting on day 61. Aspirin (100 mg/day p.o) was administered from day 53 onwards (FIG. 29). Interruption of plasma exchange for only one day while continuing ARC1779 infusion precipitated a fall in platelet counts on day 54 (dotted red circle in FIG. 28), indicating that ARC1779 is best used in conjunction with plasma exchange in such critically ill TTP patients. A loading dose of clopidogrel was given on day 57 followed by a maintenance dose of 75 mg/day (which effectively decreased P2Y12 receptor dependent signalling, as measured by the vasodilator protein phosphorylation [VASP] assay). On day 60, the ARC1779 infusion was stopped due to lack of further drug supply. At this time, a cumulative dose of approximately 10 g ARC1779 had been given during 26 days of therapy. Platelet counts declined to 3/nL within 20 hours after stop of ARC1779 infusion (FIG. 28), and creatinine and LDH values increased again (FIG. 29).

On day 87, after 91 plasma exchange treatments, the patient achieved “full recovery” of his platelet counts to the normal range (159/nL). However, ADAMTS13 activity remained undetectable (<12 U) continuously until day 103, but on day 130 levels of 0.2 U/mL were measured, while anti-ADAMTS13-antibody titer continuously dropped to <1 U/mL on day 130. At this time, the neurologic state had completely recovered.

### Pharmacokinetic/Pharmacodynamics

PK/PD modeling of data from a previous Phase I study indicated that the dose/concentration (FIG. 29) and concentration/activity response curves for ARC1779 were quite predictive of the data observed in this patient with severe, refractory TTP. Plasma concentrations of 10 μg/mL ARC1779 were associated with a ≧96% decrease in vWF-activity (assay limit of detection=4%). A sigmoid E-max model indicated that the EC₅₀ for vWF inhibition was in the range of 0.25-0.74 Hg/mL and the EC₉₀ was 1.56-3.08 μg/mL. As the volume of distribution of ARC1779 approximates the plasma volume, plasma exchange treatment acutely decreased the plasma concentrations of ARC1779 by an average of 47% (range 40-61%). Therefore, additional mini-bolus infusions of ARC1779 were given after each plasma exchange in order to restore steady-state therapeutic concentrations rapidly.

Platelet counts increased during each ARC1779 infusion period (FIGS. 28 & 29); even more convincingly, platelet counts dropped sharply after each stop of ARC1779 infusion (indicated by arrows in FIG. 29), consistent with the short half life (2 h) of ARC1779. This repeated pattern upon re-challenge provides a proof of concept that ARC1779 increases platelet counts in a patient with refractory TTP when given in addition to plasma exchange. The drop in platelet counts after stop of ARC1779 infusion was associated with a progression in organ damage (increase in creatinine levels and LDH, FIG. 29). Together with the course of the neurologic state of our patient, this provides some evidence that ARC1779 might have an impact on organ dysfunction. However, causality cannot be assigned unless tested in a randomized, placebo controlled clinical trial.

Conclusions: ARC1779 was well-tolerated and caused a clear and reproducible rise in platelet counts in an otherwise refractory TTP case. The induced platelet response alleviated severe thrombocytopenia and thus potentially reduced the risk of bleeding. This effect, which was reproducible under serial “re-challenge”, was not seen with combination therapy of aspirin plus clopidogrel. Together with the observed improvement in neurologic function, the data from this “N-of-One experiment” provide clinical proof-of-concept and suggest that ARC1779 treatment might improve the organ dysfunction that typically occurs in acute TTP. These clinical, pharmacokinetic and pharmacodynamic data provide a rational basis for clinical trials with ARC1779 in TTP.

### Example 7

### Pharmacokinetic Profiles and Pharmacokinetic and Pharmacodynamic

Relationship of a vWF Inhibitory Aptamer in the Cynomolgus Monkey The vWF aptamer ARC1779 is a 5′-20 KDa-PEGylated aptamer directed against the A1 domain of human von Willebrand Factor (vWF). ARC1779 is used as an anti-platelet agent for inhibition of vWF-mediated thrombus formation. The studies described herein were designed to determine the pharmacokinetic (PK) profile, and the pharmacokinetic and pharmacodynamic (PK/PD) relationship of ARC1779 in cynomolgus monkeys.

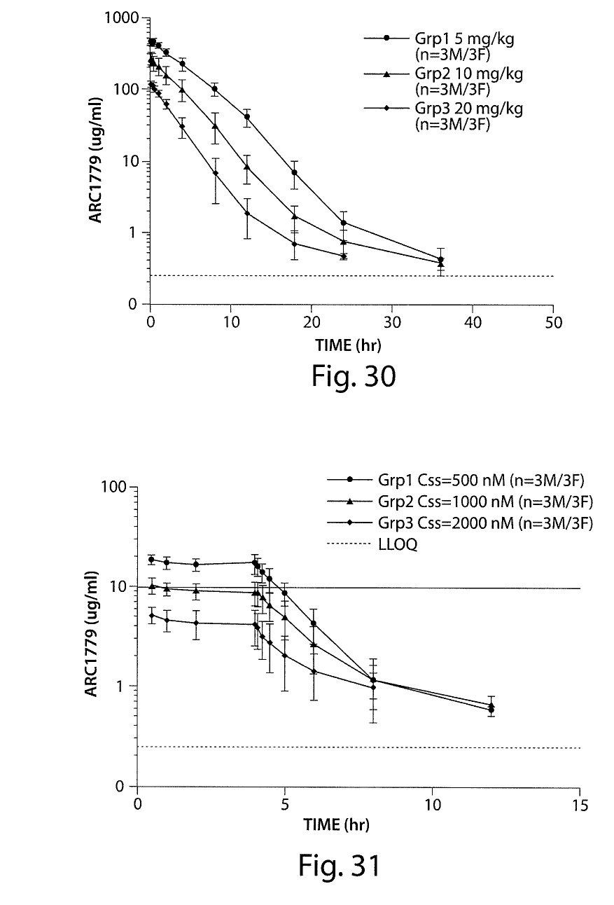

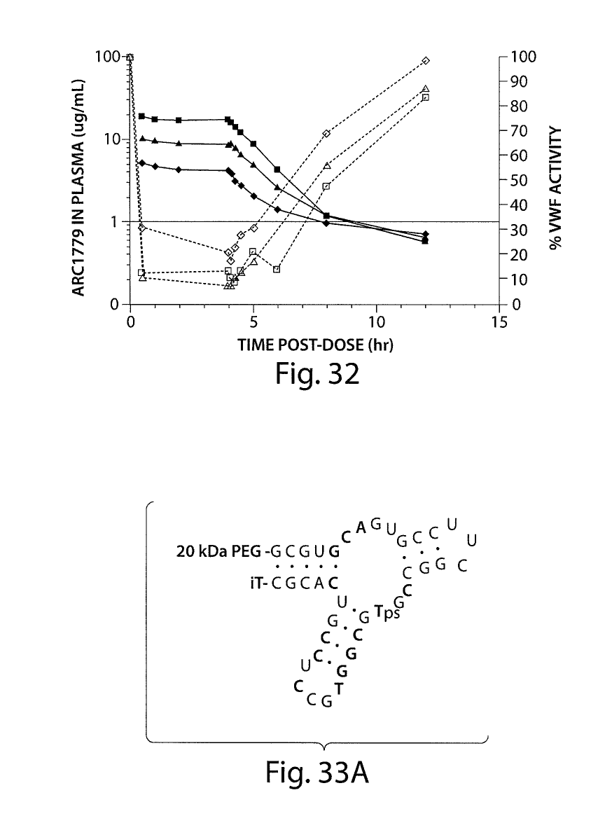

Female and male monkeys were assigned to 3 treatment groups (3M/3F each). Monkeys received single IV doses of 5, 10 or 20 mg/kg, followed by a two week washout, then an IV bolus+a continuous infusion for 4-hrs to give a total dose of 0.6, 1.2 and 2.4 mg/kg (FIGS. 30 and 31). Blood samples were obtained pre-dose and at various points post-dose. Plasma concentration was determined by HPLC. Inhibition of vWF activity was measured by a platelet function analyzer (PFA-100®) and READDS™ vWF activity ELISA KIT (FIG. 32). vWF plasma concentrations were determined by IMUBIND® vWF ELISA KIT. Noncompartmental PK parameter estimates were determined from individual plasma concentration-time data using WINNONLIN™.

TABLE 6
Study Design for IV Bolus Phase

| Phase/ | | Dose | Dose | Dose Volume | Animals | |
|---|---|---|---|---|---|---|
| Group | Route | Level | Conc. | (ml/kg) | M | F |
| 1/1 | IV | 5 | 5 | 1 | 3 | 3 |
| 1/2 | IV | 10 | 10 | 1 | 3 | 3 |
| 1/3 | IV | 20 | 20 | 1 | 3 | 3 |

TABLE 7
Study Design for IV Bolus Plus Continuous Infusion Phase

| Phase/ | | Css | | Dose_{bolus} | Dose_{infusion} | Animals | |
|---|---|---|---|---|---|---|---|
| Group | Route | nM | ug/ml | (mg/kg) | (mg/kg) | M | F |
| 2/1 | IV + IV | 500 | 6.57 | 0.200 | 1 | 3 | 3 |
| | Infusion | | | | | | |
| 2/2 | IV + IV | 1000 | 13.14 | 0.394 | 1 | 3 | 3 |
| | Infusion | | | | | | |
| 2/3 | IV + IV | 2000 | 26.28 | 0.783 | 1 | 3 | 3 |
| | Infusion | | | | | | |

Following IV bolus or IV bolus+4-hr infusion, Cmax, AUC₀₋ₗₐₛₜ and AUC_{0-∞} values increased linearly and proportionally with dose. Following a bolus dose at 5, 10 and 20 mg/kg, the Cmax values were 118.00, 281.56 and 494.58 μg/mL, the AUC₀₋ₗₐₛₜ values were 374.49, 1083.69 and 2487.25 μg·hr/mL, with elimination t½ values of 4.33, 5.58 and 4.35 hrs, respectively. Following IV bolus+4-hr infusion, the mean plasma concentrations during infusion were 4.54, 9.35 and 17.39 μg/mL with AUC₀₋ₗₐₛₜ values of 23.06, 50.11 and 90.12 μg·hr/mL for the total dose of 0.6, 1.2 and 2.4 mg/kg, respectively. Following a single IV bolus or a single IV bolus+4-hr infusion in monkeys, the PK/PD relationship of ARC1779 plasma concentrations to the EC₉₀ for inhibition of platelet function by PFA-100® were 5.81 and 3.28 μg/mL, respectively. The PK/PD relationship of ARC1779 plasma concentration with respect to the inhibition of vWF activity by the activity ELISA were 5.95 and 4.25 μg/mL, respectively. vWF plasma concentration was not effected by ARC1779.

TABLE 8
Mean Non-Compartmental Pharmacokinetic Analysis of
ARC1779 Following Single IV Bolus Administration

| PK | | 5 mg/kg | 10 mg/kg | 20 mg/kg |
|---|---|---|---|---|
| Parameters | units | (M & F) | (M & F) | (M & F) |
| t_{1/2} | hr | 4.33 | 5.58 | 3.87 |
| Cₘₐₓ | μg/mL | 118.00 | 281.56 | 494.58 |
| NCₘₐₓ | kg · μg/mL · mg | 23.60 | 28.16 | 24.73 |
| AUC₀₋ₗₐₛₜ | μg · hr/mL | 374.49 | 1083.69 | 2487.25 |
| AUC_{0-∞} | μg · hr/mL | 377.34 | 1086.97 | 2490.33 |
| NAUC_{0-∞} | kg · μg · hr/mL · mg | 75.47 | 108.70 | 124.52 |
| V_{z} | mL/kg | 83.38 | 80.37 | 46.54 |
| Cl | mL/hr/kg | 13.74 | 9.85 | 8.15 |

TABLE 9
Mean Non-Compartmental Pharmacokinetic Analysis of ARC1779
Following IV Bolus Plus Continuous Infusion Administration

| PK | | 500 nM | 1000 nM | 2000 nM |
|---|---|---|---|---|
| Parameters | units | (M & F) | (M & F) | (M & F) |
| t_{1/2} | hr | 1.79 | 1.61 | 1.22 |
| Cₘₐₓ | μg/mL | 5.27 | 10.44 | 19.09 |
| NCₘₐₓ | kg · μg/mL · mg | 8.84 | 8.80 | 8.06 |
| AUC₀₋ₗₐₛₜ | μg · hr/mL | 23.06 | 50.11 | 90.12 |
| AUC_{0-∞} | μg · hr/mL | 25.95 | 52.00 | 91.29 |
| NAUC_{0-∞} | kg · μg · hr/mL · mg | 43.55 | 43.84 | 38.57 |
| V_{z} | mL/kg | 56.88 | 51.75 | 45.54 |
| Cl | mL/hr/kg | 29.05 | 24.70 | 26.89 |

The study showed ARC1779 PK profiles were linear and proportional with increase in dose following a single IV bolus or IV bolus+4 hrs infusion, and ARC1779 blocked vWF dependent platelet function in a predictable concentration dependent manner characterized by a typical Eₘₐₓ model.

### Example 8

### Inhibition of Platelet Function by Anti-vWF Aptamer

von Willebrand Factor (vWF) interacts with platelet GPIb to facilitate adhesion and subsequent aggregation of platelets. After vascular injury, an essential first step in primary hemostasis is platelet adhesion, which leads to platelet activation and subsequent platelet aggregation via platelet GPIIb/IIIa interactions with fibrinogen, causing thrombus formation. vWF promotes platelet adhesion by binding to collagen in exposed vascular subendothelium via the A3 domain and to platelet GPIb via the A1 domain.

The studies presented herein were designed to determine the effect of the anti-vWF aptamer, ARC1779, vWF-dependent and vWF-independent platelet activation and aggregation.

ARC1779 is a highly potent and specific inhibitor of vWF activity, and vWF-mediated platelet adhesion and aggregation. With normal human samples in vitro, ARC1779 displayed comparable concentration-dependent activity in 3 different measures of vWF inhibition (PFA-100e, activity ELISA and BIPA). With normal human samples in vitro, the IC₉₀ values were 3.72, 1.15 and 4.52 μg/mL (˜0.286, 0.088 and 0.348 μM), respectively, for the ELISA, PFA-100® and BIPA assays. In human samples, maximal prolongation of PFA-100® closure times (≧300 seconds) was achieved at ˜1 μg/mL (˜0.08 μM). Despite potent inhibition of vWF-mediated platelet aggregation, ARC1779 (up to 130 μg/mL or 10 μM) had no effect on the vWF-independent platelet aggregation induced by platelet agonists, such as epinephrine, arachidonic acid, ADP, collagen and thrombin.

### Example 9

### A Phase 2 Pilot Study of the Pharmacokinetics and Pharmacodynamics of ARC1779 Injection in Patients with von Willebrand Factor-Related Platelet Function Disorders

The study objectives are: to establish the overall tolerability of ARC1779 injection in patients with von Willebrand Factor (vWF)-related platelet function disorders, including patients with thrombotic thrombocytopenic purpura (TTP) in remission, patients experiencing a current episode of acute TTP, patients with familial TTP and patients with von Willebrand Disease Type-2b (vWD-2b); to characterize the pharmacodynamic (PD) profile of ARC1779 in patients with vWF-related platelet function disorders with respect to parameters of platelet function and vWF activity; to characterize the pharmacokinetic (PK) and PD profiles of ARC1779 following subcutaneous (SC) injection; and to assess the concentration- and dose-response relationships among ARC1779 PD and PK parameters.

### Study Design

ARC1779 injection will be investigated in five cohorts of TTP patients as an uncontrolled, open-label study at a single clinical site. Patients with vWD-2b will be enrolled in one additional cohort in a randomized, blinded, double-dummy and placebo-controlled study.

Collectively, patients representing three different vWF-related platelet function disorders: TTP in remission, acute TTP and vWD-2b will be treated in a total of six cohorts. Three cohorts will consist of patients who are status post an episode of TTP (“TTP Remission Cohort 1, 2 and 3”) and will be treated by IV infusion of ARC1779 injection in a dose- and duration-escalation design. In parallel, a single cohort of patients with acute TTP (“Acute TTP Cohort 4”) will be treated by IV infusion of ARC1779 injection according to an individual patient titration-to-response paradigm. This cohort will be opened for enrollment at the beginning of the study and closed after all of the other cohorts are completed. Also in parallel, one cohort of patients with familial TTP (“Familial TTP Cohort 6”) that manifest chronic/recurrent thrombocytopenia requiring regular plasma infusion therapy will be treated by repeated-dose subcutaneous (SC) administration of ARC1779 injection. Approximately two patients are to be enrolled in each of the TTP Remission Cohorts and in the Familial TTP Cohort. Patients are to be enrolled into the Acute TTP Cohort as they become available for treatment at the clinical site. Finally, a single cohort of patients with vWD-2b (“vWD-2b Cohort 5”) will begin enrollment at the commencement of the study and continue independently of the course of the TTP Remission Cohorts. vWD-2b patients will be treated by IV infusion of ARC1779 injection, desmopressin (desmopressin is a synthetic analogue of the natural hormone arginine vasopressin that stimulates release of endothelial vWF), or their combination in a randomized, double-dummy treatment sequence. This cohort is will consist of approximately six vWD-2b patients.

ARC1779 injection will be administered to patients in TTP Remission Cohorts 1, 2 and 3 and Acute TTP Cohort 4 via weight-based dosing regimens that are intended to produce target ARC1779 plasma concentrations of 6 or 12 mcg/mL. These regimens will consist of a loading dose of ARC1779 injection given over 30 minutes as a stepwise infusion in which the rate of drug administration is increased gradually in 10-minute increments. The loading dose will be followed immediately by continuous infusion of ARC1779 injection for 4 hours or 24 hours.

Patients in Acute TTP Cohort 4 are expected to initiate treatment with ARC1779 injection for 24 hours at the 6 mcg/mL target concentration. Continuous infusion of ARC1779 injection in acute TTP patients may continue for ≦14 days and may be increased in order to achieve the 12 mcg/mL target concentration depending upon the patient's clinical and laboratory response.

Patients in Familial TTP Cohort 6 will be treated initially with an IV dose of ARC1779 injection given as a 30-minute stepwise infusion. One hour after completion of IV administration, patients will initiate a course of treatment of once-daily SC injection for ≦14 days. Treatment with ARC1779 injection will continue for 12 days at a fixed daily SC dose (50 mg) intended to result in an ARC1779 plasma concentration of approximately 3 to 10 mcg/mL. The SC dose of ARC1779 injection will be tapered by 50% on Day 13, and again by 50% on Day 14, the last day of treatment [note: if required for patient compliance or tolerability, daily SC administration of the full dose (50 mg) of ARC1779 Injection may be discontinued after Day 7 and the dose tapered by 50% daily over the next two consecutive days].

ARC1779 injection or placebo will be administered to patients in the vWD-2b Cohort in combination with a 30-minute infusion of desmopressin or placebo in a block-randomized, three-period crossover, double-dummy treatment sequence. Patients will receive a 30-minute stepwise infusion plus continuous 4-hour infusion of ARC1779 Injection (as in TTP Remission Cohort 1) in combination with a dummy 30-minute infusion of placebo, a 30-minute infusion of desmopressin in combination with a dummy 30-minute stepwise infusion plus continuous 4-hour infusion of placebo, or a 30-minute stepwise infusion plus continuous 4-hour infusion of ARC1779 injection in combination with 30-minute infusion of desmopressin.

### Study Population

The study will include patients with prior episodes of primary acute TTP, patients with primary or secondary forms of acute TTP and patients with vWD-2b.

### Investigational Product, Mode(s) of Administration, and Dose(s)

### Investigational Product:

ARC1779 injection

### Mode(s) of Administration:

IV: Stepwise infusion of a weight-based dose (mg/kg) over a period of 30 minutes followed by continuous infusion of a weight-based dose (mg/kg) for durations of 4 hours, 24 hours or ≦14 days.
SC: Repeated fixed doses (mg) given by once-daily SC injection for ≦14 days.

### Dose(s):

The total doses of ARC1779 injection to be administered are as follows: Cohort 1, 0.47 mg/kg; Cohort 2, 1.67 mg/kg; Cohort 3, 3.34 mg/kg; Cohort 4, 40.78 mg/kg; Cohort 5, 0.47 mg/kg; Cohort 6, ˜656 mg (for an 80 kg patient).

### Study Treatments

The TTP Remission Cohorts will follow a pre-specified sequence of escalation of total dose and/or duration of infusion of ARC1779 injection. The first of these cohorts, TTP Remission Cohort 1, will receive a total dose of 0.47 mg/kg of ARC1779 as an initial stepwise infusion of 0.23 mg/kg given over 30 minutes and subsequent continuous infusion of an additional 0.24 mg/kg given over 4 hours at a rate of 0.001 mg/kg/min. TTP Remission Cohort 2 will receive a total dose of ARC1779 injection of 1.67 mg/kg as an initial stepwise infusion of 0.23 mg/kg given over 30 minutes and subsequent continuous infusion of an additional 1.44 mg/kg given over 24 hours at a rate of 0.001 mg/kg/min. The dosing regimens for TTP Remission Cohorts 1 and 2 are intended to achieve a target ARC1779 plasma concentration of 6 mcg/mL for 4 hours and 24 hours, respectively. TTP Remission Cohort 3 will receive a total dose of ARC1779 injection of 3.34 mg/kg given as an initial stepwise infusion of 0.46 mg/kg over 30 minutes and subsequent continuous infusion of an additional 2.88 mg/kg given over 24 hours at a rate of 0.002 mg/kg/min. The dosing regimen for TTP Remission Cohort 3 is intended to achieve a target ARC1779 plasma concentration of 12 mcg/mL for 24 hours.

The Acute TTP Cohort will include patients with an ongoing episode of acute TTP as they become available at the study site. It is anticipated that patients in the Acute TTP Cohort will initiate treatment with ARC1779 injection for 24 hours according to the dosing regimen that will produce a target ARC1779 plasma concentration of 6 mcg/mL (i.e., the same regimen used for TTP Remission Cohort 2). The continuous infusion may be continued in acute TTP patients for ≦14 days. After initiation of ARC1779 injection infusion at the established rate, the dose may be titrated to achieve a target ARC1779 plasma concentration of 12 mcg/mL as needed, on the basis of clinical and laboratory data, according to the investigator's judgment.

Familial TTP Cohort 6 will receive an initial stepwise infusion of ARC1779 injection at 0.23 mg/kg given over 30 minutes (as in Cohort 2). One hour after completion of stepwise infusion of ARC1779 injection, patients will be given a SC injection of 50 mg of study drug. SC administration of 50 mg of ARC1779 injection will be repeated once per day as a 12-day course of treatment. This SC dosing regimen is expected to result in an ARC1779 plasma concentration of 3 to 10 mcg/mL. The daily SC dose of ARC1779 injection will be tapered by 50% on Day 13 (i.e. to 25 mg) and by 50% again on Day 14 (i.e. to 12.5 mg).

The vWD-2b Cohort will be treated according to a three-period, crossover, double-dummy design with ARC1779 injection or placebo, desmopressin or placebo, or their combination given in a block-randomized sequence. In one period of the sequence, Period X, ARC1779 injection will be administered to all members of the cohort at a total dose of 0.47 mg/kg given as a stepwise infusion of 0.23 mg/kg over 30 minutes and subsequent continuous infusion of an additional 0.24 mg/kg given over 4 hours at a rate of 0.001 mg/kg/min (the same regimen as given to TTP Remission Cohort 1) in combination with a dummy 30-minute infusion of placebo. In another period, Period Y, patients will receive a single infusion of desmopressin at a dose of 0.4 mcg/kg given over 30 minutes in combination with a dummy 30-minute stepwise infusion plus 4-hour continuous infusion of placebo. In the one other period of the sequence, Period Z, patients will receive the combination of desmopressin (as in Period Y) and ARC1779 injection (as in Period X).

### Criteria for Evaluation

### PK Variables:

Plasma samples will be analyzed for quantification of ARC1779 concentrations using a validated high performance liquid chromatography (HPLC) method.

### PD Variables:

The PD variables to be measured in this Phase 2 study are: platelet count, vWF activity (by ELISA directed toward the A1 domain), vWF antigen, vWF multimer gel electrophoresis, shear-dependent platelet function (PFA-100®), shear-dependent platelet adhesion (cone and plate analyzer) and multiplate platelet function analysis.

### Study Duration

Patients in TTP Remission Cohorts 1, 2 and 3 will participate in the study for up to approximately 3 weeks, including a Screening Period (Day-14 to Day-1) prior to dosing, Treatment (Day 1) and Follow-up Visits on Day 2, Day 3 (TTP Remission Cohort 2 and Cohort 3 only) and Day 8. Patients in Acute TTP Cohort 4 and Familial TTP Cohort 6 will participate in the study for up to approximately 4 weeks, including a Pre-dose Assessment and Treatment Period (Day 1 to Day 14) and Follow-up Visits between Day 21 and 28. Patients in vWD-2b Cohort 5 will participate in the study for up to approximately 5-7 weeks, including a Screening Period (Day-14 to Day-1) and 3 single-day treatments, with 1-week washout in between each treatment.

### Example 10

### Inhibition of vWF-mediated Platelet Activation and Thrombosis by ARC1779, an Anti-vWF A1-Domain Aptamer

ARC1779 binds to the A1-domain of vWF with high affinity and potently inhibits vWF-dependent platelet aggregation. ARC1779 was able to reduce platelet accumulation and adhesion in human whole blood. In addition, human platelet thrombus formation on denuded porcine arteries was reduced. ARC1779 inhibited formation of occlusive thrombi in cynomolgus macaques during electrical injury to the carotid arteries throughout a 6-hour intravenous infusion, while demonstrating reduced template bleeding relative to similarly protective anti-glycoprotein GPIIb/IIIa inhibitor, abciximab.

### Methods

### Oligonucleotide Synthesis and PEG Conjugation

Oligonucleotides were synthesized on either an EXPEDITE® 8909 DNA/RNA Synthesizer (ABI, Foster City, Calif.) (≦1 μmole scale) or on an AKTA OLIGOPILOT® (Amersham Pharmacia Biotech, Piscataway N.J.) (≧1 μmole scale) using standard phosphoramidite solid-phase chemistry. All phosphoramidites were acquired from Chemgenes Corporation (Wilmington, Mass.). A subset of aptamers was synthesized with a hexylamine moiety at the 5′-end and conjugated to high molecular weight PEGs from Nippon Oil and Fat (NOF) (White Plains, N.Y.) post-synthetically via amine-reactive chemistry. The resulting products were purified by ion exchange and reverse phase HPLC.

### Nitrocellulose Filter Binding Assays

For binding assays, the oligonucleotide core of ARC1779 was synthesized with a 5′-terminal hydroxyl moiety instead of a 5′ hexylamine and 20 kDa PEG and subsequently 5′-end labeled with γ-³²P ATP using T4 polynucleotide kinase (New England BioLabs, Beverly, Mass.). Aptamer was incubated at room temperature for 30 minutes in PBS containing 0.1 mg/ml BSA (as a specificity control) and increasing concentrations of human VWF. Bound complexes were separated from free DNA using an acrylic mini-dot blot apparatus (Schleicher and Schuell, Keene, N.H.) and a membrane sandwich (top to bottom) of Protran BA85 0.45 μm Nitrocellulose Membrane (Schleicher and Schuell, Keene, N.H.), Hybond P Nylon Membrane (Amersham, Piscataway, N.J.), and 3mM filter paper (Whatman, Florham Park, N.J.) all pre-wet with Dulbecco's PBS. Radioactivity associated with protein:DNA complexes (nitrocellulose) and free DNA (nylon) was quantified on a Storm 860 Phosphorimager (Molecular Dynamics/Amersham, Piscataway, N.J.). Binding constants were determined by fitting the fraction of aptamer bound as a function of the concentration of vWF.

### Adhesion of Platelets to Collagen Associated vWF

A parallel plate with a 0.0127 cm silicon rubber gasket (Glycotech) coated with 100 μg/mL collagen (Nycomed) was used. A 20 mL blood sample was collected from healthy donors into 90 μM PPACK. Washed platelets were obtained from 10 mL of blood and labeled with 2.5 μg/mL calcein orange, then added back to the whole blood. Blood was treated with various concentrations of ARC1779 at 37° C. (10 nM-500 nM). Platelet adhesion was monitored with an Axiovert 135 inverted microscope (Zeiss) at 32× and silicon-intensified tube camera C 2400 (Hamamatsu). Adhesion was analyzed with Image SXM 1.62 (NIH Image).

### Thrombus Formation on Injured Porcine Artery

A 120 ml sample of venous human blood from normal volunteers was anti-coagulated with PPACK (90 ml) or ACD (30 ml). Porcine aorta segments were isolated, dissected free of surrounding tissues, cut into rings and longitudinally opened. Injured segments were prepared by lifting and peeling off the intima to expose the subjacent media. The segments were placed into Badimoon perfusion chambers with a 1 mm internal diameter. The chambers were placed in parallel (two per side) in a thermostatically controlled water bath (at 37° C.), thus permitting simultaneous parallel, pair wise perfusion over arterial tissues of treated or untreated blood at high shear (6974/sec in 1 mm ID chambers). In the experiments, blood was perfused and recirculated over the arterial segments, for 10 min followed by an additional 5 min without (control) or with abciximab (100 nM), or ARC1779 (150 nM, 600 nM or 1.5 μM). At the end of the perfusion, the surfaces were immediately fixed in tissue fix followed by SEM examination of thrombus mass on the different surfaces.

### Platelet Function Assays

Blood was drawn into 0.105 M sodium citrate by venipuncture from healthy donors who had not taken non-steroidal anti-inflammatory drugs for at least 3 days. Platelet-rich plasma (PRP) and platelet-poor plasma (PPP) were prepared from the collected blood by centrifugation. PRP was aliquoted into cuvettes containing stir bars at a volume of 470 μl. A sample of 500 μl of PPP was aliquoted into a cuvette and placed in the PPP reference cell of the model 490-4D platelet aggregometer (Chronolog, Havertown, Pa.). Samples of PRP were pre-warmed at 37° C. in the platelet aggregometer for 3-5 minutes before being used in aggregation assays. For botrocetin-induced platelet aggregation (BIPA) reactions, 1.5 U of botrocetin was used per 500 μl reaction. Test articles were then assayed by diluting increasing concentrations of aptamer into pre-warmed PRP, incubating the mixture for 1 minute and then adding botrocetin. The platelet aggregometer monitored changes in light transmission over 6 minutes post addition of the agonist.

The platelet function analyzer 100 (PFA-100®) (Dade Behring, Deerfield, Ill.) was used to assay the shear force-dependent anti-platelet activity of aptamers in whole blood. Test article or vehicle was added to citrated whole blood from healthy volunteers (obtained as described above) and was assayed for anti-platelet activity by using collagen/epinephrine cartridges according the manufacturer's protocols. Inhibition of shear force-dependent platelet function was correlated with the time to occlusion of the cartridge aperture with a maximum measurable time of 300 seconds.

### Analysis of Aptamer Concentrations in Cynomolgus Macaque Plasma

Blood samples were obtained at selected time points during all animal studies via saphenous venipuncture, transferred directly to EDTA-coated tubes and placed on wet ice. Plasma was harvested by centrifugation of blood-EDTA. Plasma samples were stored at −80° C. Samples were analyzed for aptamer concentration by ion-exchange HPLC (Agilent 1100 series, Santa Clara, Calif.) with UV detection at 256 nm using a DNAPak PA-100 column (4×250 mm) (Dionex, Sunnyvale, Calif.). Sample concentrations were determined by interpolation of integrated sample peak area at the known retention time from a standard curve generated using reference standards of known concentration. The HPLC methods were qualified with a lower limit of quantitation (LLOQ) of approximately 0.02 μM and a linear range of 0.02 to 50 μM.

### Template Bleeding Time in Cynomolgus Macaques

Using a SURGICUTT® Automated Incision Device (ITC, Edison, N.J.), a longitudinal incision was made over the lateral aspect of the volar surface of the forearm. Bleeding times were measured according to the manufacturer's instructions. In cases where bleeding time would have exceeded 15 minutes, the wound was closed with a suture. Bleeding time was determined to within 30 seconds of the time when blood no longer wicked onto the paper.

### Electrical Injury Model of Arterial Thrombosis

The electrical injury model of arterial thrombosis was performed on 22 cynomolgus macaques according to the methods of Rote et al. Electrolytic injury to the intimal surface of each carotid artery was accomplished via a continuous current delivered to each vessel via an intravascular electrode for a period of 3 hours or for 30 minutes after complete occlusion, whichever was shorter. Control animals received a saline injection and infusion for 6 hours. Doses of ARC1779 and abciximab are summarized in Table 15. Approximately 15 minutes after test article administration, the electrical current was applied at 100 μA. The current was terminated either ˜30 minutes after the blood flow signal remained stable at zero flow (indicating the formation of an occlusive thrombus at the site) or after 180 minutes of electrical stimulation without occlusion, whichever occurred first. Approximately 195 minutes after onset of the test article infusion, the left carotid artery was subjected to electrical injury in a similar fashion.

### Statistical Analysis

Data from the electrical injury model were analyzed using a non-parametric one-way ANOVA® (Kruskal-Wallis Test) followed by Dunn's Test using the program GraphPad InStat version 3.05 for Windows 95/NT (GraphPad Software, San Diego, Calif.).

### Results

### Aptamer In Vitro Pharmacology

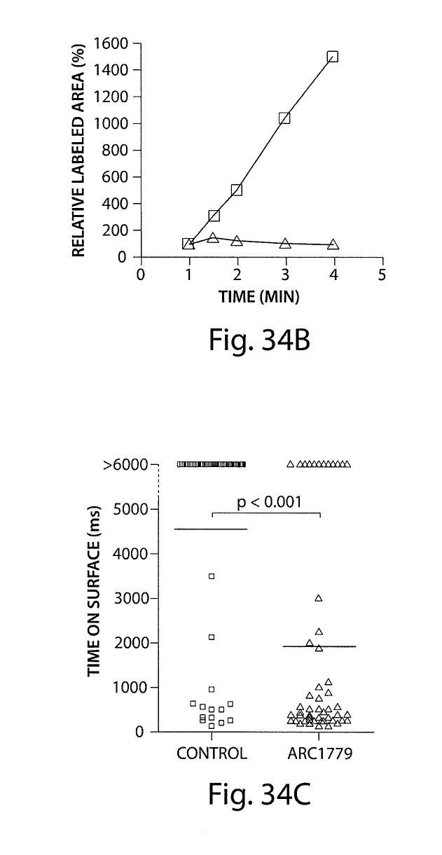

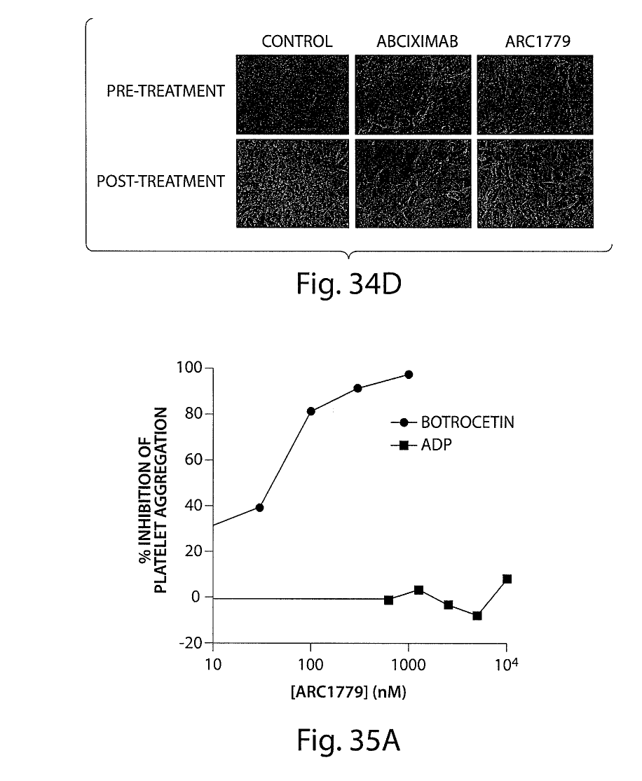

Binding of fluorescently labeled platelets mixed into human whole blood to a collagen coated surface was measured in the absence and presence of ARC1779. The percent area with fluorescent platelets was quantified after 2 minutes of blood flow at shear rate of 1,500/sec. Approximately 40% of the surface was covered with adherent platelets in the control group. Consistent with a binding affinity of ARC1779 for vWF of ˜2 nM and an average concentration of vWF in blood of 30-50 nM, the lowest concentrations of ARC1779 tested (10 nM and 25 nM) reduced the covered surface area by ˜50%. Higher concentrations of 75 to 400 nM, which likely exceed the vWF concentration in human whole blood reduced covered area by >90% (FIG. 34A). Next, untreated blood was perfused over the surface for 1 minute followed by up to 4 minutes of continued perfusion with either untreated or ARC1779 treated (200 nM) blood. Although adherent platelets were not displaced, the continued accumulation of platelets onto the surface of the adherent platelets was significantly reduced though the entire 4 minutes perfusion period (FIG. 34B). More importantly, when initial tethering and firm adhesion (dwell time) was evaluated at the 400 nM ARC1779 concentration, it was found that platelet dwell time of platelets in the ARC1779 treated samples was significantly reduced suggesting that ARC1779 should be beneficial in reducing platelet thrombus formation (FIG. 34C). Finally, human whole blood was perfused over injured porcine arteries for 15 minutes at a 6,974/sec shear rate. Untreated samples had significant platelet accumulation and thrombus formation, as measured by scanning electron microscopy. Abciximab treated blood (100 nM) and ARC1779 treated blood (150 nM, 600 nM and 1.5 uM) both had significant reduction of platelet accumulation and thrombus formation on the injured artery surface (FIG. 34D).

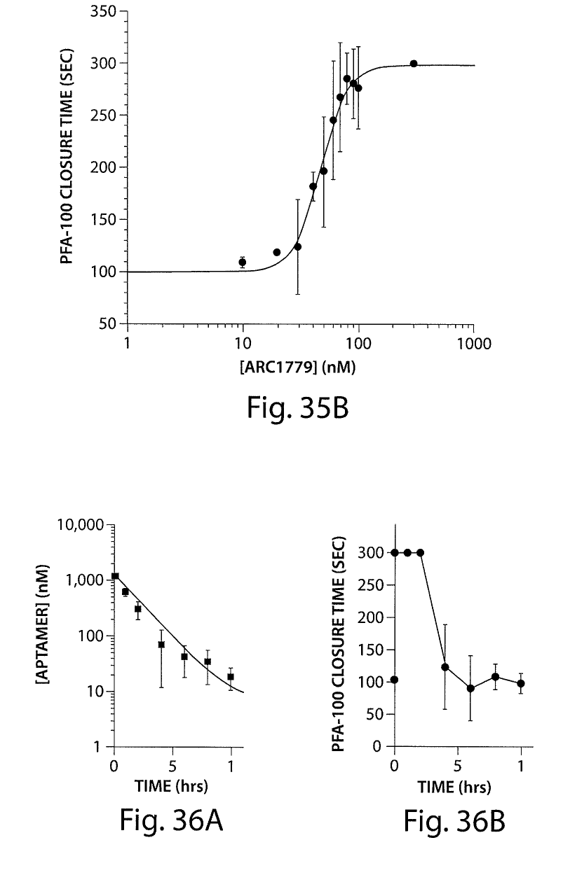

Two assays that measure the functional activity of vWF were used to test the ability of aptamers to neutralize. vWF-dependent platelet activation. The first assay is the botrocetin-induced platelet aggregation assay (BIPA). As shown in FIG. 35A, ARC1779 is a potent inhibitor of BIPA with IC₉₀ values of 100-300 nM, but consistent with its anti-vWF mode of action, shows no ability to inhibit ADP induced platelet activation. The second measure of aptamer functional activity was with the platelet function analyzer (PFA-100®). The PFA-100® simulates the formation of a hemostatic plug under conditions of high shear force in vivo by recording the time required for platelets to aggregate and block the flow of blood through a microscopic aperture in a membrane coated with collagen and epinephrine or ADP. As shown in FIG. 35B, ARC1779 inhibits vWF-dependent platelet activity, as measured by PFA-100® closure time in vitro.

### Pharmacokinetic/Pharmacodynamic Studies

Next, we established the pharmacokinetic/pharmacodynamic relationship for ARC1779. As shown in FIG. 36A, dosing in cynomolgus macaques at 0.5 mg/kg resulted in a plasma Cₘₐₓ of ˜1 M and a pharmacokinetic half-life of 1.4 hrs. The pharmacokinetic profile is similar to that which is reported for eptifibatide, and may offer a potential advantage with respect to the bleeding risk in patients for ACS patients who may require urgent coronary artery bypass surgery. Furthermore, as shown in FIG. 36B and consistent with in vitro data shown in FIG. 36B, as long as the plasma level of ARC1779 remained above 100 nM, vWF-dependent platelet activity, as assessed by PFA-100® closure time, was completely inhibited (300 seconds is the maximum time measured by the instrument). However, when the ARC1779 plasma levels dropped below 100 nM, the anti-platelet effect rapidly returned to baseline levels.

### Efficacy in an Electrical Injury Model of Arterial Thrombosis

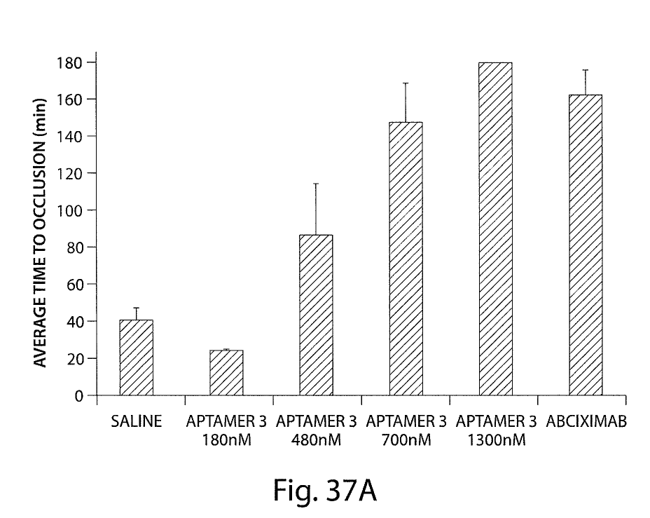

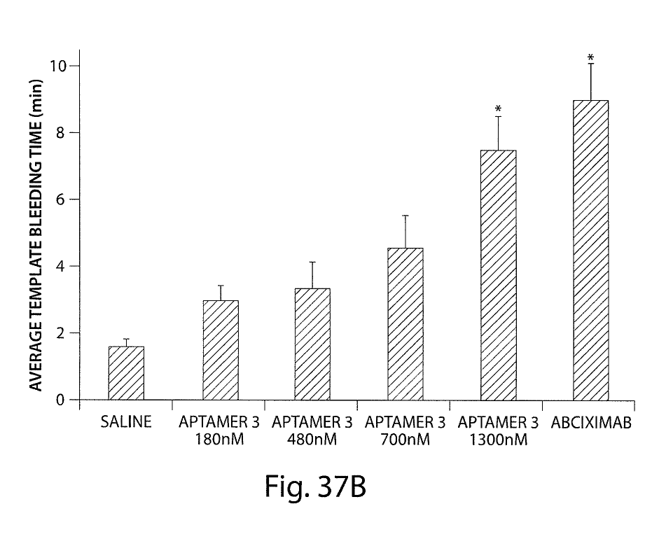

As summarized in Table 10 and FIG. 37A, ARC1779 showed clear dose-dependent efficacy in a cynomolgus macaque model of electrical injury (EI). Using an intravenous bolus+continuous infusion dosing regimen (0.3 mg/kg bolus+0.0025 mg/kg/min infusion) that achieved mean plasma concentrations of 700 nM and 1300 nM, ARC1779 demonstrated efficacy comparable or superior to that of a previously published dose of abciximab with respect to protection from thrombus formation and average time to occlusion (Table 10 and FIG. 37A). These data suggest that 700 nM is a pharmacologically effective target plasma concentration for ARC1779.

Consistent with the dose-dependent efficacy of ARC1779 in the EI model, there was also a dose-dependent increase in template bleeding time. However, as shown in FIG. 37B, analysis of average template bleeding times during the course of these experiments revealed that while treatment with a plasma concentration of 1300 nM ARC1779 or abciximab significantly prolonged template bleeding times relative to saline control animals (P<0.001), a plasma concentration of 700 nM ARC1779 did not.

### Discussion

ARC1779 potently inhibits the interaction between the vWF A1 domain and the platelet receptor GPIb, and inhibits vWF-dependent platelet activation.

Using collagen coated plates, ARC1779 effectively reduced the adhesion of platelets to blood borne vWF that accumulated on the collagen surface in a concentration dependent manner. Concentrations of ARC1779 as low as 10 nM reduced platelet adhesion vs. control by 50% and at concentrations >100 nM adhesion was reduced by >90%. ARC1779 exposure reduced the adhesion time of individual platelets suggesting that exposure to ARC1779 could reduce thrombus growth. This was confirmed in the flow chamber studies with damaged porcine arteries. ARC1779 at concentrations as low as 150 nM reduced platelet adhesion and thrombus growth as demonstrated by platelet accumulation and analysis using scanning electron microscopy. The in vitro efficacy of ARC1779 was equivalent to a therapeutic concentration of the GPIIb/IIIa antagonist abciximab. Thus to further define the efficacy of ARC1779 and GPIb/vWF inhibition vs GPIIb/IIIa inhibition, we extended the comparison of ARC1779 and abciximab to a cynomolgus monkey thrombosis model.

It has been speculated that based upon the difference in mechanism of action, vWF antagonists may offer intrinsic advantages over GPIIb/IIIa antagonists with respect to therapeutic ratio and bleeding risk. However, the earlier anti-vWF compounds, AJW200 and GPG-290, were not tested in the more stringent primate model of EI-induced arterial thrombosis utilized here and in the proof of concept study for abciximab, one which entails three hours of continuous electrical injury to the vessel wall. Here we have shown by direct comparison in this model that ARC1779 has an efficacy profile similar to that of abciximab yet significantly reduced template bleeding, substantiating the postulated advantage of vWF antagonism over GPIIb/IIIa antagonism.

TABLE 10

| | | | | | Mean | Number | Protection |
|---|---|---|---|---|---|---|---|
| | | | | Mean | PFA-100 | of | from |
| | Bolus | Infusion | Number | Aptamer | closure | Occlusive | Occlusive |
| | Dose | Dose | of | [Plasma] | time | Arterial | Thrombus |
| Treatment | (ug/kg) | (ug/kg/min) | Animals | (nM) | (sec) | Thrombi** | Formation (%) |
| Saline | NA* | NA | 3 | NA | 90 | 6/6 | 0 |
| | | | | | (±10) | | |
| (ARC1779) | 100 | 1 | 2 | 180 | 300 | 4/4 | 0 |
| | | | | (±20) | | | |
| (ARC1779) | 200 | 1.6 | 4 | 480 | 300 | 5/8 | 37 |
| | | | | (±90) | | | |
| (ARC1779) | 300 | 2.5 | 4 | 700 | 300 | 2/8 | 75 |
| | | | | (±100) | | | |
| (ARC1779) | 600 | 3.7 | 5 | 1300 | 300 | 0/10 | 100 |
| | | | | (±400) | | | |
| Abciximab | 250 | 1.3 | 5 | NA | 300 | 2/10 | 80 |

*NA, Not applicable;
**Two (left and right) carotid arteries were tested per animal.

### Example 11

### Combination Therapy

This example shows in vivo pharmacology studies in cynomolgus monkeys for ARC1779 used in combination with various drugs. In these studies, the in vivo pharmacologic activity and PK/PD relationships of ARC1779 were evaluated. The assays used to quantify ARC1779-related activity included: vWF antigen (total vWF) concentrations in plasma by enzyme-linked immunosorbent assay (ELISA); vWF activity in plasma (ELISA); vWF-mediated, high shear force-dependent platelet adhesion and aggregation in whole blood (platelet function), as assessed with a platelet function analyzer (PFA-100®); and cutaneous bleeding time, as measured with the use of a template surgical device.

In pharmacodynamic drug interaction studies, ARC1779 in vitro (up to 130 μg/mL) had no effect on ACT values in the presence (up to 3 U/mL) or absence of heparin. ARC1779 administered to cynomolgus monkeys in a dose-escalating fashion over 4 hours alone or in combination with aspirin, clopidogrel, unfractionated heparin, low molecular weight heparin or bivalirudin resulted in expected plasma ARC1779 concentrations and ARC1779-related inhibition of vWF activity that were not affected by any of the other antithrombotics. ACT values were not affected by ARC1779 and PFA-100® values were affected (slightly earlier increase) only by concurrent administration of clopidogrel. Cutaneous bleeding times were markedly prolonged when ARC1779 was administered with clopidogrel, UFH, LMWH or bivalirudin, but not when administered alone or with aspirin. Thus, there were no unexpected pharmacodynamic drug interactions when ARC1779 was administered in combination with other antithrombotic agents.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating, preventing or ameliorating a disease mediated by von Willebrand Factor (vWF) comprising administering to a subject in need thereof a therapeutically effective dose of an aptamer that binds to vWF and has the following structure: 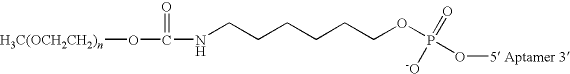
wherein: “n” is about 454 ethylene oxide units (PEG=20 kDa), and the aptamer comprises the following nucleic acid sequence or fragment thereof: mGmCmGmUdGdCdAmGmUmGmCmCmUmUmCmGmGmCdCmG-s-dTmGdCdGdGdTmGmCdCmUdCdCmGmUdCmAmCmGmC-3T (SEQ ID NO: 1), and further wherein “m” is a 2′-OMe substituted nucleotide, “d” is a deoxyribonucleotide, “s” is a phosphorothioate internucleotide linkage and “3T” is an inverted deoxythymidine, wherein the aptamer is administered at a dose in the range of 0.05 mg/kg to 10 mg/kg.

2. The method of claim 1, wherein the vWF-mediated disease is a thrombotic disease.

3. The method of claim 2, wherein the thrombotic disease is selected from the group consisting of: acute coronary syndrome, thrombotic microangiopathies, thrombotic thrombocopenic purpura, von Willebrand Disease type 2b and transient ischemic attack.

4. The method of claim 1, wherein said administration is intravenous.

5. The method of claim 1, wherein said administration is subcutaneous.

6. The method of claim 1, wherein the dose is 5 mg/kg.

7. The method of claim 1, wherein the dose achieves a steady state blood concentration of 2-12 μg/ml.

8. The method of claim 7, wherein the steady state blood concentration is 3-6 μg/ml.

9. The method of claim 1, wherein the formulation is administered in combination with a different dosage form of the same formulation.

10. The method of claim 1, wherein the formulation is administered in combination with another drug.

11. The method of claim 10, wherein the another drug is selected from the group consisting of: corticosteroids, antihistamines and immunosuppressives.

12. The method of claim 11, wherein the another drug is aspirin or clopidogrel.

13. The method of claim 1, wherein the formulation is administered in combination with another therapy.

14. The method of claim 13, wherein the therapy is selected from the group consisting of: plasma exchange and angioplasty.

15. A formulation comprising:
a) an aptamer that binds to vWF comprising the following structure: 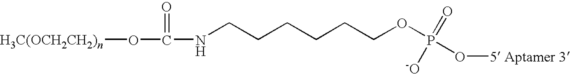
wherein: “n” is about 454 ethylene oxide units (PEG=20 kDa), and the aptamer comprises the following nucleic acid sequence or fragment thereof: mGmCmGmUdGdCdAmGmUmGmCmCmUmUmCmGmGmCdCmG-s-dTmGdCdGdGdTmGmCdCmUdCdCmGmUdCmAmCmGmC-3T (SEQ ID NO: 1), and further wherein “m” is a 2′-OMe substituted nucleotide, “d” is a deoxyribonucleotide, “s” is a phosphorothioate internucleotide linkage and “3T” is an inverted deoxythymidine; and
b) a pharmaceutically acceptable solvent,
wherein the formulation comprises 0.5-2000 mg of aptamer.

16. The formulation of claim 15, wherein the formulation comprises 1 ml of solvent.

17. The formulation of claim 15, wherein the formulation comprises 10 mg of aptamer per 1 ml of solvent.

18. The formulation of claim 15, wherein the formulation is aqueous.

19. The formulation of claim 15, wherein the vWF aptamer is lyophilized.

20. The formulation of claim 15, wherein the formulation is packaged in a pharmaceutically acceptable container.
